(19) United States
(12) Patent Application Publication
(10) Pub. No.: US 2026/0060957 A1
(43) Pub. Date: Mar. 5, 2026

(54) COMPOUNDS AND COMPOSITIONS USEFUL FOR TREATMENT OF PROLIFERATIVE DISEASE OR DISORDER

(71) Applicants: University of Houston System, Houston, TX (US); Tract Pharmaceuticals, Inc., West Hartford, CT (US)

(72) Inventors: Wa Xian, Sugar Land, TX (US); Frank McKeon, Sugar Land, TX (US); Matthew Vincent, Amesbury, MA (US); Richard E. Russell, Scituate, MA (US)

(21) Appl. No.: 19/308,852

(22) Filed: Aug. 25, 2025

Related U.S. Application Data
(63) Continuation of application No. PCT/US2024/052562, filed on Oct. 23, 2024.
(60) Provisional application No. 63/592,886, filed on Oct. 24, 2023.
(60) Provisional application No. 63/631,430, filed on Apr. 8, 2024.
(60) Provisional application No. 63/634,874, filed on Apr. 16, 2024.
(60) Provisional application No. 63/654,667, filed on May 31, 2024.

(51) Int. Cl. A61K 31/4025; A61K 9/00; A61K 31/4192; A61K 31/5025; A61P 35/00

(52) U.S. Cl. CPC A61K 31/4025; A61K 9/0053; A61K 31/4192; A61K 31/5025; A61P 35/00

(57) ABSTRACT

The present disclosure provides compounds, pharmaceutically acceptable compositions thereof and methods of using the same.

### BACKGROUND OF THE INVENTION

There is a continuing need to identify therapeutic modalities for treatment of disease (e.g., cancer and pulmonary diseases). Treatment of disease by combination therapy has demonstrated some effectiveness. However, there remains a substantial unmet medical need for treatments that are more effective for treating a variety of diseases.

### SUMMARY OF THE INVENTION

The present disclosure recognizes that treatment of diseases with a unique combination of inhibitors is particularly effective. Notably, the present disclosure recognizes that a combination therapy comprising inhibitors of cellular kinases and inhibitors of Inhibitors of Apoptosis (IAPs) is uniquely effective in various diseases.

In some embodiments, the present disclosure provides inhibitors of IAPs useful as therapeutic agents in combination with inhibitors of cellular kinases. It has now been found that compounds of the present disclosure, and pharmaceutically acceptable salts and compositions thereof, are effective as inhibitors of IAPs and effective in treatment of various diseases in combination with cellular kinases.

In some embodiments, the present disclosure provides a compound of formula I:

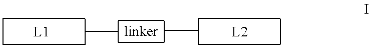

or a pharmaceutically acceptable salt thereof, wherein: L1 is a first ligand; L2 is a second ligand; and linker is a bivalent linker comprising

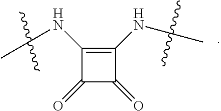 [SMILES: *Nc1c(N*)c(=O)c1=O]

Compounds described herein, and pharmaceutically acceptable compositions thereof, are useful for treating a variety of diseases when provided in combination with inhibitors of cellular kinases to a subject suffering from a disease.

### BRIEF DESCRIPTION OF THE DRAWING

FIG. 1 demonstrates efficacy of combinations of the present disclosure for the treatment of cystic fibrosis.

FIG. 2 demonstrates efficacy of combinations of the present disclosure for the treatment of asthma.

FIG. 3 demonstrates efficacy of combinations of the present disclosure for the treatment of COPD.

FIG. 4 demonstrates efficacy of combinations of the present disclosure for the treatment of idiopathic pulmonary fibrosis.

FIG. 5 demonstrates efficacy of combinations of the present disclosure for the treatment of pancreatic cancer.

FIGS. 6A and 6B demonstrates efficacy of combinations of the present disclosure for the treatment of pancreatic cancer.

FIGS. 7A and 7B demonstrates efficacy of combinations of the present disclosure for the treatment of pancreatic cancer.

FIG. 8 shows reduction in FAP (red) in pancreatic cells treated with TP101.

FIG. 9 shows reduction in the number of clonogenic pancreatic cancer cells when treated with TP101.

FIGS. 10A-10C, 11, 12A-12C, and 13 demonstrates efficacy of combinations of the present disclosure for the treatment of esophageal cancer.

FIGS. 14A-14C and 15 demonstrate efficacy of combinations of the present disclosure for the treatment of gastric cancer.

FIGS. 16, 17, 18, and 19 demonstrate efficacy of combinations of the present disclosure for the treatment of ovarian cancer.

FIGS. 20, 21, 22, and 23 demonstrate efficacy of combinations of the present disclosure for the treatment of glioblastoma and neuroglioma.

FIG. 24 demonstrates efficacy of combinations of the present disclosure for the treatment of lung cancer.

FIG. 25 demonstrates efficacy of combinations of the present disclosure for the treatment of liver cancer.

FIG. 26 demonstrates efficacy of combinations of the present disclosure for the treatment of colon cancer.

FIG. 27 demonstrates efficacy of combinations of the present disclosure for the treatment of pharyngeal cancer.

FIGS. 28 and 29 demonstrate efficacy of combinations of the present disclosure for the treatment of breast cancer.

FIGS. 30 and 31 demonstrate combinations of the present disclosure are non-toxic.

FIGS. 32a-32f demonstrate stem cells from precursor lesions. 32a. From left, White-light imaging of distal esophagus depicting biopsy sites of co-existing mucosal lesions. EAC, esophageal adenocarcinoma; HGD, high-grade dysplasia; LGD, low-grade dysplasia; BE, Barrett's; ESO, normal esophagus. 32b. Generation of single cell-derived clones in 384-well plates via FACS-aided single cell from indicated biopsy. 32c. Phase-contrast image of colonies from expanded, single cell-derived clones. Scale bar: 200 um. 32d. Immunofluorescence from sections of epithelia derived from air-liquid interface (ALI) differentiation of discrete clones of BE, LGD, HGD, and EAC showing distribution of antibodies to E-cadherin (ECAD, red) and the proliferation marker Ki67 (green). Scale bar: 50 um. 32e. Histological sections of nodules derived from BE, LGD, HGD, and EAC stem cell xenografts in immunodeficient mice. Scale bar: 100 um. 32f Graphical representation of nodule growth following xenografting of indicated stem cell colones to immunodeficient mice. Error bars, SD.

FIGS. 33a-33b demonstrates histology of present precursor lesions. 33a. White light endoscopic imaging of distal esophagus in Case 1 with incisor distances noted. 33b. Top, Histology of endoscopic biopsies adjacent to those used to generate libraries of BE, LGD, HGD, and EAC of Case 1. Bottom, Histology of endoscopic biopsies adjacent to those used to generate libraries of BE, LGD, HGD, and EAC of Case 2. Scale bar: 100 um.

FIGS. 34a-34c demonstrates histology of present precursor lesions 34a. Immunofluorescence micrographs of single cell-derived colonies stained with antibodies to human nuclei (red) and to CDH17 (green). Scale bar: 100 um. 34b. Histogram of clonogenicity data from stem cell culture, ALI cultures, or xenograft nodules from stem cells at in vitro passage 5 and at passage 25. 34c. Histology (left) and Sox9, ECAD, and GPA33 immunofluorescence (right) of sections of epithelia derived from in vitro-differentiated in air-liquid interface cultures of BE, LGD, HGD, and EAC stem cells. Scale bar: 100 um.

FIGS. 35a-35i demonstrate clone variation and genomic stability of lesional stem cells. 35a. Copy number variation (CNV) profiles of 79 clones sampled from indicated biopsy libraries determined from low-pass, whole genome sequencing. Deletions, blue; Amplifications, red. 35b. Copy number profiles of selected clones determine by exome sequencing. c. Histogram of allele frequency distribution for all somatic single nucleotide mutations across 35 clones from Case 1. 35d. Percentage overlap of SNV events among clones derived from the indicated biopsies. 35e. Copy number profiles of chromothripsis events on chromosome 16 in single HGD and EAC clones of Case 1. 35f Schematic for analysis of genetic stability of EAC clone through serial passaging in vitro and following tumor formation in mice. 35g. Copy number profile of EAC clone C1D1-7 determined from whole exome sequencing. 35h. Copy number variation profiles of EAC clone C1D1-7 following in vitro propagation and xenografting for tumor formation in mice. 35I. Variant allele fraction profiles of subclones presented in h.

FIGS. 36a-36f demonstrates phylogenetics of patient-matched lesional stem cells. 36a. Phylogenetic tree of 35 cloned stem cell lineages assembled from 679 somatic SNVs (AF>0.2). Positions of sustained mutations impacting CDKN2A, ARID1A, ERBB2, TP53, and other genes are indicated. In-frame deletion, green; frame-shift, purple; stop-gain, red; nonsynonymous, blue. CT16, chromothripsis of Chromosome 16. 36b. Heatmap reflecting variant allele fraction of the 679 somatic SNVs. 36c. GISTIC2.0 identified significant amplified focal regions (red) in clones from indicated lesions. 36d. Copy number profiles focused on the ERBB2 locus of chromosome 17 and an estimated copy number of ERBB2 across indicated clones. 36e. Phylogenetic tree of 45 patient-matched stem cell clones from biopsies of a second EAC case based on 462 somatic SNVs (AF>0.2). Positions of sustained mutations impacting CDKN2A, ARID1A, ERBB2, TP53, and other genes are indicated. In-frame deletion, green; frame-shift, purple; stop-gain, red; nonsynonymous, blue; CT8, chromothripsis of chromosome 8 are indicated. 36f Heatmap of variant allele fraction of the 462 somatic SNVs.

FIG. 37 demonstrates phylogenetics of patient-matched lesional stem cells. Phylogenetic tree depicting the relationships between cloned stem cells of Case 1 BE, LGD, HGD, and EAC together with the associated somatic mutations at each transition. In-frame deletions, green; frame-shift, purple; nonsynonymous, blue; stop-gain, red. Genes listed at 90 degrees on the right are mutations sustained by the indicated clone after the emergence of the next more advanced lesion.

FIGS. 38a-38b demonstrates copy number variations of precursor lesions described herein 38a. Selected copy number variation events in clones of Case 1 indexed by the indicated gene affecting oncogenes (left) or tumor suppressor genes (right). 38b. Selected copy number variation events in clones of Case 2 indexed by the indicated gene affecting oncogenes (left) or tumor suppressor genes (right).

FIGS. 39a-39c demonstrates phylogenetics of patient-matched lesional stem cells 39a. A phylogenetic tree depicting the relationships between cloned stem cells of Case 2 BE, LGD, HGD, and EAC together with the associated somatic mutations at each transition. In-frame deletions, green; frame-shift, purple; nonsynonymous, blue; stop-gain, red. Genes listed at 90 degrees on the right are mutations sustained by the indicated clones after the emergence of the next more advanced lesion. 39b. Recurrent CNV amplification events across 24 clones selected from BE, LGD, HGD, and EAC of Case 2. Amplifications are labeled by index genes within the amplicon. 39c. Recurrent CNV deletion events across 24 clones selected from BE, LGD, HGD, and EAC of Case 2 labeled by index genes within the deletion.

FIGS. 40a-40i demonstrate synthetic lethal combinations targeting BE. 40a. Schematic for parallel small molecule screens against patient-matched stem cells of BE and normal esophageal mucosa. 40b. From left, scatter plot of impact of small molecules on survival of BE stem cells versus novel, dose-response of candidate drugs from primary screen against GFP-labelled BE stem cells, and survival dose-response curves of candidate mitomycin C against BE stem cells and normal ESO stem cells. 40c. Primary screen against GFP-labelled normal ESO stem cells showing well with relative overgrowth of ESO stem cells (circled with inset). 40d. Scatter plot of drug screen survival weighted from eight patient-matched BE and normal esophageal stem cells (ESO). The most potent stimulator of normal esophageal stem cells was the tyrosine kinase inhibitor ponatinib (circled and inset). 40e. Scatter plot of drug screen survival in the presence of ponatinib (300 nM), with new candidates marked in red. 40f Dose-response curves of BE and ESO stem cells against the IAP antagonist SM-164 in the presence and absence of ponatinib (300 nM). 40g. Dose-response curves of stem cell clones from 23 cases of BE and three normal ESO stem cells against SM-164 in the presence of ponatinib (300 nM). 40h. Dose-response curves of SM-164 in the presence of ponatinib (300 nM) by stem cells of BE, LGD, HGD, and EAC from Case 1. 40i. Co-culture of normal ESO stem cells (red) with stem cells of BE, LGD, HGD, and EAC in the absence and presence of the combination of SM-164 (10 nM) and ponatinib (300 nM) for five days.

FIGS. 41a-41b demonstrate analysis of genomic alteratsion in lesions described herein 41a. Schematic aggregation of genomic alterations (non-synonymous mutations, stop-gain, indels, CNV, chromothripsis, genome duplication) sustained at each transition to more advanced lesions across two EAC cases. 41b. Graphical representation of in-line mutational events (CNV, code-altering point mutations) at each transition.

FIGS. 42a-42l provides mechanistic analyses of novel synthetic lethal combinations. 42a. Structures of monovalent and bivalent IAP antagonists. 42b. Dose-response curves of established IAP antagonists in the presence of ponatinib (300 nM) against LGD stem cells from Case 1. 42c. Western blot of established IAP antagonists (10 nM) in the presence of ponatinib (300 nM) against LGD stem cells after 16 hr. 42d. Novel series of bivalent IAP antagonists built around squaramide-polyether linkers and headgroups of AZD-5582 and SM-164. 42e. Co-cultures of normal esophageal stem cells (Krt14+, red) and stem cells of BE, LGD, HGD, and EAC (Krt7+, green) in the absence (top) and presence (bottom) of 7532N (10 nM) and ponatinib (300 nM) for five days. 42f. Western blot of normal esophageal stem cells exposed to ponatinib (300 nM) alone or in combination with 7532N (10 nM) showing the IAP antagonistic-induced loss of cIAP1 and cIAP2 but no induction of the apoptosis-associated cleavage of Caspase 3. 42g. Luminescence imaging and immunofluorescence of co-xenografts in immunodeficient mice of LGD stem cells (luciferase+, Krt7+, green) and normal esophageal stem cells (krt14+, red) treated seven times over 20 days with vehicle alone or 7532N (5 mg/kg) and ponatinib (15 mg/kg). 42h. Histogram of quantification of p63+ stem cells in Krt14+ epithelia in control and 7532N/ponatinib-treated mice at day 20. 42i. Generalize scheme of multiprotein Complex 1 linked to cellular decisions governing cell death decisions leading to apoptosis or necroptosis, with proteins that directly interact with ponatinib shown in orange. 42j. Western blot of LGD stem cells treated with indicated drug or drug combinations for 16 hr using antibodies to the indicated proteins. TKI (ponatinib, 300 nM), 7532N (10 nM), RIPK1 inhibitor (GSK2982772; 1 uM). 42k. Western blot of cleave-Caspase 3 induction in LGD stem cells by a six hour exposure to single, double, or triple combinations of ponatinib, 7532N, and established inhibitors of IKKa/IKKb, MK2, p38MAPK, or TAK1. 42l. Western blot of cleave-Caspase 3 induction in LGD stem cells by a six-hour exposure to combinations of ponatinib, 7532N, TAK1 inhibitor, and RIPK1 inhibitor.

FIGS. 43a-43d provides analyses of novel synthetic lethal combinations. 43a. Chemical structures of potential IAP antagonists synthesized using squaramide polyether linkers about AZD5582 and SM-164 headgroups. 43b. Western blots of LGD stem cells treated with synthesized IAP antagonists (10 nM) alone (left) or in combination with ponatinib (300 nM), right. 43c. Chart of data by Kauster et al., 2015 and Hnatiuk et al., 2023 related to the binding and inhibition by ponatinib or the ponatinib analog 33a analog on kinases implicated in the regulation of cell death.

FIGS. 44a-44f demonstrate synthetic lethal combination against EAC stem cells. 44a. Dose-response cures of 7532N in the presence of ponatinib (300 nM) against stem cells from eight EAC cases and two normal ESO stem cells. 44b. Luminescence imaging of xenografts of luciferase-labelld EAC stem cells during treatment with vehicle alone (top) or the combination of 7532N (5 mg/kg) and ponatinib (15 mg/kg) three times per week (bottom). Right, graphical representation of tumor size measured by caliper and corresponding animal weights over the course of the treatment. 44c. Clonogenicity assay for treated and control tumors. Left, dissected control and treated tumors. Right, 10-day growth of EAC stem cell colonies on lawns of irradiated 3T3-J2 cells imaged by antibodies to human nuclei. 44d. Quantification of EAC stem cell colonies from identical weights of treated and control xenograft nodule tissue. 44e. Histological sections of control and treated EAC stem cell xenograft nodules. Immunofluorescence labeling of control and treated tumor with antibodies to E-cadherin (ECAD, green) and the proliferation marker Ki67(red).

FIG. 45 provides cancer driver mutations in Case 1

FIG. 46 provides cancer driver mutations in Case 2

FIG. 47 provides an overview of efficacy of combinations described herein (7532-N+ponatinib) Therapy in a single mouse trial (SMT) with 27 tumor models. The minimum T/C values for all experiments are shown in the order of highest to lowest efficacy. A minimum T/C value of 65% (upper limit for borderline antitumor efficacy) was chosen as a turning point. Efficacy rating: regression: T/C<5%; very high efficacy: 5%≤T/C<10%; high efficacy: 10%≤T/C<25%; moderate efficacy: 25%≤T/C<50%; borderline efficacy: 50%≤T/C<65%; -: T/C>65%.

FIGS. 48A and 48B demonstrate antitumor efficacy of 7532-N+121-H in tumor model GXA 3002. (A) Relative tumor volume over time; (B) Relative body weight over time.

FIGS. 49A and 49B demonstrate antitumor efficacy of 7532-N+121-H in tumor model GXA 3011. (A) Relative tumor volume over time; (B) Relative body weight over time.

FIGS. 50A and 50B demonstrate antitumor efficacy of 7532-N+121-H in tumor model GXA 3012. (A) Relative tumor volume over time; (B) Relative body weight over time.

FIGS. 51A and 51B demonstrate antitumor efficacy of 7532-N+121-H in tumor model GXA 3023. (A) Relative tumor volume over time; (B) Relative body weight over time.

FIGS. 52A and 52B demonstrate antitumor efficacy of 7532-N+121-H in tumor model GXA 3027. (A) Relative tumor volume over time; (B) Relative body weight over time.

FIGS. 53A and 53B demonstrate antitumor efficacy of 7532-N+121-H in tumor model GXA 3037. (A) Relative tumor volume over time; (B) Relative body weight over time.

FIGS. 54A and 54B demonstrate antitumor efficacy of 7532-N+121-H in tumor model GXA 3039. (A) Relative tumor volume over time; (B) Relative body weight over time.

FIGS. 55A and 55B demonstrate antitumor efficacy of 7532-N+121-H in tumor model GXA 3063. (A) Relative tumor volume over time; (B) Relative body weight over time.

FIGS. 56A and 56B demonstrate antitumor efficacy of 7532-N+121-H in tumor model GXA 3087. (A) Relative tumor volume over time; (B) Relative body weight over time.

FIGS. 57A and 57B demonstrate antitumor efficacy of 7532-N+121-H in tumor model GXF 602. (A) Relative tumor volume over time; (B) Relative body weight over time.

FIGS. 58A and 58B demonstrate antitumor efficacy of 7532-N+121-H in tumor model OVXF 550. (A) Relative tumor volume over time; (B) Relative body weight over time.

FIGS. 59A and 59B demonstrate antitumor efficacy of 7532-N+121-H in tumor model OVXF 899. (A) Relative tumor volume over time; (B) Relative body weight over time.

FIGS. 60A and 60B demonstrate antitumor efficacy of 7532-N+121-H in tumor model OVXF 1320. (A) Relative tumor volume over time; (B) Relative body weight over time.

FIGS. 61A and 61B demonstrate antitumor efficacy of 7532-N+121-H in tumor model OVXF 1353. (A) Relative tumor volume over time; (B) Relative body weight over time.

FIGS. 62A and 62B demonstrate antitumor efficacy of 7532-N+121-H in tumor model OVXF 1993. (A) Relative tumor volume over time; (B) Relative body weight over time.

FIGS. 63A and 63B demonstrate antitumor efficacy of 7532-N+121-H in tumor model OVXF 2437. (A) Relative tumor volume over time; (B) Relative body weight over time.

FIGS. 64A and 64B demonstrate antitumor efficacy of 7532-N+121-H in tumor model OVXF GYN090. (A) Relative tumor volume over time; (B) Relative body weight over time.

FIGS. 65A and 65B demonstrate antitumor efficacy of 7532-N+121-H in tumor model PAXF 736. (A) Relative tumor volume over time; (B) Relative body weight over time.

FIGS. 66A and 66B demonstrate antitumor efficacy of 7532-N+121-H in tumor model PAXF 1657. (A) Relative tumor volume over time; (B) Relative body weight over time.

FIGS. 67A and 67B demonstrate antitumor efficacy of 7532-N+121-H in tumor model PAXF 2005. (A) Relative tumor volume over time; (B) Relative body weight over time.

FIGS. 68A and 68B demonstrate antitumor efficacy of 7532-N+121-H in tumor model PAXF 2035. (A) Relative tumor volume over time; (B) Relative body weight over time.

FIGS. 69A and 69B demonstrate antitumor efficacy of 7532-N+121-H in tumor model PAXF 2059. (A) Relative tumor volume over time; (B) Relative body weight over time.

FIGS. 70A and 70B demonstrate antitumor efficacy of 7532-N+121-H in tumor model PAXF 2082. (A) Relative tumor volume over time; (B) Relative body weight over time.

FIGS. 71A and 71B demonstrate antitumor efficacy of 7532-N+121-H in tumor model PAXF 2094. (A) Relative tumor volume over time; (B) Relative body weight over time.

FIGS. 72A and 72B demonstrate antitumor efficacy of 7532-N+121-H in tumor model PAXF 2116. (A) Relative tumor volume over time; (B) Relative body weight over time.

FIGS. 73A and 73B demonstrate antitumor efficacy of 7532-N+121-H in tumor model PAXF 2146. (A) Relative tumor volume over time; (B) Relative body weight over time.

FIGS. 74A and 74B demonstrate antitumor efficacy of 7532-N+121-H in tumor model PAXF 2196. (A) Relative tumor volume over time; (B) Relative body weight over time.

FIG. 75 shows dose-response assay of viability of pathogenic stem cells in Crohn's disease (inflammatory gastric metaplasia) in response to either 7532N (I-1) or 7532N-G (I-11) with either ponatinib (“121”; 300 nM) alone or with ponatinib (300 nM) and SM1-71 (20 nM).

FIG. 76 shows dose-response assay of viability of normal lung stem cells versus 7532N in the presence or absence of 500 nM ponatinib.

### DEFINITIONS

About: As used herein, the term “about” refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value. In some embodiments, “about” refers to ±10%, ±9%, ±8%, ±7%, ±6%, ±5%, ±4%, ±3%, ±2%, ±1% of a referenced value.

Administration: As used herein, the term “administration” typically refers to the administration of a composition to a subject or system, for example to achieve delivery of an agent that is, or is included in or otherwise delivered by, the composition.

Agent: As used herein, the term “agent” refers to an entity (e.g., for example, a lipid, metal, nucleic acid, polypeptide, polysaccharide, small molecule, etc., or complex, combination, mixture or system [e.g., cell, tissue, organism] thereof), or phenomenon (e.g., heat, electric current or field, magnetic force or field, etc.).

Antagonist: As used herein, the term “antagonist” may refer to an agent, or condition whose presence, level, degree, type, or form is associated with a decreased level or activity of a target. An antagonist may include an agent of any chemical class including, for example, small molecules, polypeptides, nucleic acids, carbohydrates, lipids, metals, and/or any other entity that shows the relevant inhibitory activity. In some embodiments, an antagonist may be a “direct antagonist” in that it binds directly to its target; in some embodiments, an antagonist may be an “indirect antagonist” in that it exerts its influence by means other than binding directly to its target; e.g., by interacting with a regulator of the target, so that the level or activity of the target is altered). In some embodiments, an “antagonist” may be referred to as an “inhibitor”.

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle”, “carbocyclic”, “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein the term “biological sample” includes, without limitation, cell cultures or extracts thereof, biopsied material obtained from an animal (e.g., mammal) or extracts thereof, and blood, saliva, urine, feces, semen, tears, or other body fluids or extracts thereof; or purified versions thereof. For example, the term “biological sample” refers to any solid or fluid sample obtained from, excreted by or secreted by any living organism, including single-celled micro-organisms (such as bacteria and yeasts) and multicellular organisms (such as plants and animals, for instance a vertebrate or a mammal, and in particular a healthy or apparently healthy human subject or a human patient affected by a condition or disease to be diagnosed or investigated). The biological sample can be in any form, including a solid material such as a tissue, cells, a cell pellet, a cell extract, cell homogenates, or cell fractions; or a biopsy, or a biological fluid. The biological fluid may be obtained from any site (e.g. blood, saliva (or a mouth wash containing buccal cells), tears, plasma, serum, urine, bile, seminal fluid, cerebrospinal fluid, amniotic fluid, peritoneal fluid, and pleural fluid, or cells therefrom, aqueous or vitreous humor, or any bodily secretion), a transudate, an exudate (e.g. fluid obtained from an abscess or any other site of infection or inflammation), or fluid obtained from a joint (e.g. a normal joint or a joint affected by disease such as rheumatoid arthritis, osteoarthritis, gout or septic arthritis). The biological sample can be obtained from any organ or tissue (including a biopsy or autopsy specimen) or may comprise cells (whether primary cells or cultured cells) or medium conditioned by any cell, tissue or organ. Biological samples may also include sections of tissues such as frozen sections taken for histological purposes. Biological samples also include mixtures of biological molecules including proteins, lipids, carbohydrates and nucleic acids generated by partial or complete fractionation of cell or tissue homogenates. Although the sample is preferably taken from a human subject, biological samples may be from any animal, plant, bacteria, virus, yeast, etc. The term animal, as used herein, refers to humans as well as non-human animals, at any stage of development, including, for example, mammals, birds, reptiles, amphibians, fish, worms and single cells. Cell cultures and live tissue samples are considered to be pluralities of animals. In certain exemplary embodiments, the non-human animal is a mammal (e.g., a rodent, a mouse, a rat, a rabbit, a monkey, a dog, a cat, a sheep, cattle, a primate, or a pig). An animal may be a transgenic animal or a human clone. If desired, the biological sample may be subjected to preliminary processing, including preliminary separation techniques.

Combination therapy: As used herein, the term “combination therapy” refers to those situations in which a subject is concomitantly exposed to two or more therapeutic regimens (e.g., two or more therapeutic agents). In some embodiments, the two or more regimens may be administered concomitantly; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein, the phrase “corresponding to” refers to a relationship between two entities, events, or phenomena that share sufficient features to be reasonably comparable such that “corresponding” attributes are apparent. For example, in some embodiments, the term may be used in reference to a compound or composition, to designate the position and/or identity of a structural element in the compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen or therapeutic regimen: Those skilled in the art will appreciate that the terms “dosing regimen” and “therapeutic regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Excipient: As used herein, the term “excipient” refers to a non-therapeutic agent that may be included in a pharmaceutical composition, for example, to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Improved, increased or reduced: As used herein, the terms “improved,” “increased,” or “reduced,”, or grammatically comparable comparative terms thereof, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.).

Oral: The phrases “oral administration” and “administered orally” as used herein have their art-understood meaning referring to administration by mouth of a compound or composition.

Parenteral: The phrases “parenteral administration” and “administered parenterally” as used herein have their art-understood meaning referring to modes of administration other than enteral and topical administration, usually by injection, and include, without limitation, intravenous, intramuscular, intra-arterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

Patient or subject: As used herein, the term “patient” or “subject” refers to any organism to which a provided composition is or may be administered, e.g., for experimental, diagnostic, prophylactic, cosmetic, and/or therapeutic purposes. Typical patients or subjects include animals (e.g., mammals such as mice, rats, rabbits, non-human primates, and/or humans). In some embodiments, a patient is a human. In some embodiments, a patient or a subject is suffering from or susceptible to one or more disorders or conditions. In some embodiments, a patient or subject displays one or more symptoms of a disorder or condition. In some embodiments, a patient or subject has been diagnosed with one or more disorders or conditions. In some embodiments, a patient or a subject is receiving or has received certain therapy to diagnose and/or to treat a disease, disorder, or condition.

Pharmaceutical composition: As used herein, the term “pharmaceutical composition” refers to an active agent, formulated together with one or more pharmaceutically acceptable carriers. In some embodiments, the active agent is present in unit dose amounts appropriate for administration in a therapeutic regimen to a relevant subject (e.g., in amounts that have been demonstrated to show a statistically significant probability of achieving a predetermined therapeutic effect when administered), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and/or animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Prevent or prevention: As used herein, the terms “prevent” or “prevention”, when used in connection with the occurrence of a disease, disorder, and/or condition, refer to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Small molecule: As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer.

In some embodiments, a small molecule does not include a polymeric moiety. In some embodiments, a small molecule is not and/or does not comprise a protein or polypeptide (e.g., is not an oligopeptide or peptide). In some embodiments, a small molecule is not and/or does not comprise a polynucleotide (e.g., is not an oligonucleotide). In some embodiments, a small molecule is not and/or does not comprise a polysaccharide; for example, in some embodiments, a small molecule is not a glycoprotein, proteoglycan, glycolipid, etc.). In some embodiments, a small molecule is not a lipid.

In some embodiments, a small molecule is a modulating agent (e.g., is an inhibiting agent or an activating agent). In some embodiments, a small molecule is biologically active. In some embodiments, a small molecule is detectable (e.g., comprises at least one detectable moiety). In some embodiments, a small molecule is a therapeutic agent.

Those of ordinary skill in the art, reading the present disclosure, will appreciate that certain small molecule compounds described herein may be provided and/or utilized in any of a variety of forms such as, for example, crystal forms (e.g., polymorphs, solvates, etc), salt forms, protected forms, pro-drug forms, ester forms, isomeric forms (e.g., optical and/or structural isomers), isotopic forms, etc.

Those of ordinary skill in the art will appreciate that certain small molecule compounds have structures that can exist in one or more steroisomeric forms. In some embodiments, such a small molecule may be utilized in accordance with the present disclosure in the form of an individual enantiomer, diastereomer or geometric isomer, or may be in the form of a mixture of stereoisomers; in some embodiments, such a small molecule may be utilized in accordance with the present disclosure in a racemic mixture form.

Those of skill in the art will appreciate that certain small molecule compounds have structures that can exist in one or more tautomeric forms. In some embodiments, such a small molecule may be utilized in accordance with the present disclosure in the form of an individual tautomer, or in a form that interconverts between tautomeric forms.

Those of skill in the art will appreciate that certain small molecule compounds have structures that permit isotopic substitution (e.g., ²H or ³H for H; ¹¹C, ¹³C or ¹⁴C for ¹²C; ¹³N or ¹⁵N for ¹⁴N; ¹⁷O or ¹⁸O for ¹⁶O; ³⁶Cl for ^{35/37}Cl; ¹⁸F for ¹⁹F; ¹³¹I for ¹²⁷I; etc). In some embodiments, such a small molecule may be utilized in accordance with the present disclosure in one or more isotopically modified forms, or mixtures thereof.

In some embodiments, reference to a particular small molecule compound may relate to a specific form of that compound. In some embodiments, a particular small molecule compound may be provided and/or utilized in a salt form (e.g., in an acid-addition or base-addition salt form, depending on the compound); in some such embodiments, the salt form may be a pharmaceutically acceptable salt form.

In some embodiments, where a small molecule compound is one that exists or is found in nature, that compound may be provided and/or utilized in accordance in the present disclosure in a form different from that in which it exists or is found in nature. Those of ordinary skill in the art will appreciate that, in some embodiments, a preparation of a particular small molecule compound that contains an absolute or relative amount of the compound, or of a particular form thereof, that is different from the absolute or relative (with respect to another component of the preparation including, for example, another form of the compound) amount of the compound or form that is present in a reference preparation of interest (e.g., in a primary sample from a source of interest such as a biological or environmental source) is distinct from the compound as it exists in the reference preparation or source. Thus, in some embodiments, for example, a preparation of a single stereoisomer of a small molecule compound may be considered to be a different form of the compound than a racemic mixture of the compound; a particular salt of a small molecule compound may be considered to be a different form from another salt form of the compound; a preparation that contains only a form of the compound that contains one conformational isomer ((Z) or (E)) of a double bond may be considered to be a different form of the compound from one that contains the other conformational isomer ((E) or (Z)) of the double bond; a preparation in which one or more atoms is a different isotope than is present in a reference preparation may be considered to be a different form; etc.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Treat: As used herein, the terms “treat,” “treatment,” or “treating” refer to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example, for the purpose of decreasing the risk of developing pathology associated with the disease, disorder, and/or condition.

Therapeutically effective amount: As used herein, the term “therapeutically effective amount” refers to an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder and/or condition. In some embodiments, a therapeutically effective amount is administered in a single dose; in some embodiments, multiple unit doses are required to deliver a therapeutically effective amount.

It is understood by one skilled in the art that compounds referred to herein may be enriched at any or all atoms above naturally occurring isotopic ratios with one or more isotopes such as, but not limited to, deuterium (²H or D).

The compounds of the disclosure, or their pharmaceutically acceptable salts, may contain chiral centers, which, unless specified otherwise, may be either of the (R) or (S) configuration, or which may comprise a mixture thereof. Accordingly, the present application includes stereoisomers of the compounds described herein, where applicable, either individually or admixed in any proportions. Stereoisomers may include, but are not limited to, enantiomers, diastereomers, racemic mixtures, and combinations thereof. Such stereoisomers can be prepared and separated using conventional techniques, either by reacting enantiomeric starting materials, or by separating isomers of compounds of the present application.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

As used herein, the term “partially unsaturated”, as used herein, refers to a ring moiety that includes at least one double or triple bond. The term “partially unsaturated”, as used herein, is intended to encompass rings having multiple sites of unsaturation, but is not intended to include aryl or heteroaryl moieties, as herein defined.

The term “lower alkyl”, as used herein, refers to a C₁₋₄ straight or branched alkyl group. Exemplary lower alkyl groups are methyl, ethyl, propyl, isopropyl, butyl, isobutyl, and tert-butyl.

The term “halogen” means F, Cl, Br, or I.

The term “aryl”, as used herein, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments, “aryl” refers to an aromatic ring system which includes, but not limited to, phenyl, biphenyl, naphthyl, anthracyl and the like, which may bear one or more substituents. Also included within the scope of the term “aryl” is a group in which an aromatic ring is fused to one or more non-aromatic rings, such as indanyl, phthalimidyl, naphthimidyl, phenanthridinyl, or tetrahydronaphthyl, and the like.

The term “heteroaryl” as used herein, refers to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” as used herein, refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, tetrahydroquinolinyl, and tetrahydroisoquinolinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples of heteroaryl rings on compounds of Formula I and subgenera thereof include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted.

Additionally, it will be appreciated that, when two groups cyclize to form an optionally substituted heteroaryl ring having at least one nitrogen atom, the nitrogen atom in the ring can be, as valency permits, N or N—R^{†}, as defined infra.

As used herein, the terms “heterocycle”, “heterocyclyl”, and “heterocyclic ring” are used interchangeably and refer to a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, tetrahydroquinolinyl, or tetrahydroisoquinolinyl where the radical or point of attachment is on the heterocyclyl ring. A heterocyclyl group may be mono- or bicyclic.

Additionally, it will be appreciated that, when two groups cyclize to form an optionally substituted heterocyclic ring having at least one nitrogen atom, the nitrogen atom in the ring can be, as valency permits, N or N—R^{†}, as defined infra.

As described herein, compounds may contain “optionally substituted” moieties. In general, the term “substituted”, whether preceded by the term “optionally” or not, means that one or more hydrogens of the designated moiety of compounds are replaced with a suitable substituent. “Substituted” applies to one or more hydrogens that are either explicit or implicit from the structure (e.g.,

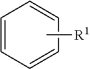

refers to at least

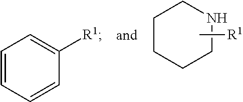

refers to at least

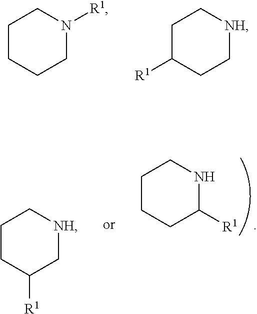

Unless otherwise indicated, an “optionally substituted” group may have a suitable substituent at each substitutable position of the group, and when more than one position in any given structure may be substituted with more than one substituent selected from a specified group, the substituent may be either the same or different at every position.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR⁰²; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5- to 6 membered heteroaryl ring), or a 5- to 6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group of a compound of Formula I, and subgenera thereof, include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{•} include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5- to 6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of RI are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur.

Pharmaceutically Acceptable Salt As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the present disclosure. Unless otherwise stated, all tautomeric forms are within the scope of the disclosure. Additionally, unless otherwise stated, the present disclosure also includes compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure. In some embodiments, compounds of this disclosure comprise one or more deuterium atoms.

Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

### DETAILED DESCRIPTION OF CERTAIN EMBODIMENTS

The present disclosure recognizes the efficacy of a combination of inhibitors of IAP and inhibitors of cellular kinases in the treatment of various proliferative diseases and disorders. The present disclosure provides inhibitors of IAPs. The present disclosure recognizes that combination of inhibitors of IAPs described herein with tyrosine kinase inhibitors is particularly effective in treatment of various proliferative diseases and disorders.

### 1. Inhibitors of Inhibitors of Apoptosis (IAPs):

In certain embodiments, the present disclosure provides inhibitors of IAPs. In some embodiments, an IAP is selected from BIRC1/NAIP, BIRC2/cIAP1, BIRC3/cIAP2, BIRC4/XIAP, BIRC5/Survivin, BIRC6/Apollon, BIRC7/ML-IAP and BIRC8/ILP2.

IAP (Inhibitor of apoptosis) proteins, a family of anti-apoptotic proteins, have an important role in evasion of apoptosis, as they can both block apoptosis-signaling pathways and promote survival. Eight members of this family have been described in humans (BIRC1/NAIP, BIRC2/cIAP1, BIRC3/cIAP2, BIRC4/XIAP, BIRC5/Survivin, BIRC6/Apollon, BIRC7/ML-IAP and BIRC8/ILP2). In certain embodiments, the agent is an IAP Inhibitor (i.e., an IAP Antagonist). Exemplary IAP Inhibitors include XIAP inhibitors, CIAP inhibitors, and agents acting as dual XIAP and CIAP inhibitors.

Exemplary IAP inhibitors and antagonists include Birinapant (a bivalent Smac mimetic, which is a potent antagonist for XIAP and cIAP1 with Kds of 45 nM and less than 1 nM, respectively), LCL161 Inhibitor (an IAP inhibitor which inhibits XIAP and cIAP1 with IC₅₀'s of 35 and 0.4 nM), AZD5582 (AZD5582 an IAP antagonist which binds to the BIR3 domains cIAP1, cIAP2, and XIAP), SM-164 (a cell-permeable Smac mimetic compound that binds to XIAP protein containing both the BIR2 and BIR3 domains with an IC₅₀ value of 1.39 nM and functions as an extremely potent antagonist of XIAP), BV6 (an antagonist of cIAP1 and XIAP), Xevinapant (or AT-406, is a potent and orally bioavailable Smac mimetic and an antagonist of IAPs, and it binds to XIAP, cIAP1, and cIAP2 proteins), GDC-0152 (a potent IAPs inhibitor, and binds to the BIR3 domains of XIAP, cIAP1, cIAP2 and the BIR domain of ML-IAP), ASTX660 (an orally bioavailable dual antagonist of cIAPs and XIAPs), CUDC-427 (a potent second-generation pan-selective IAP antagonist), Embelin (or Embelic acid, a potent, nonpeptidic XIAP inhibitor). APG-1387 (a bivalent SMAC mimetic and an IAP antagonist, blocks the activity of IAPs family proteins (XIAP, cIAP-1, cIAP-2, and ML-IAP), MX69 (an inhibitor of MDM2/XIAP), MV1, Polygalacin D, UC-112, AZD5582 dihydrochloride, HY-125378m Tolinapant (ASTX660) and SBP-0636457.

In some embodiments, compounds disclosed herein bind to one or more IAPs (e.g., NAIP, cIAP1, cIAP2, XIAP, Survivin, Apollon, ML-IAP, or ILP2). In some embodiments, compounds disclosed herein inhibitor activity or one or more IAPs (e.g., NAIP, cIAP1, cIAP2, XIAP, Survivin, Apollon, ML-IAP, or ILP2).

The activity of a compound described herein as an inhibitor of one or more IAPs (e.g., NAIP, cIAP1, cIAP2, XIAP, Survivin, Apollon, ML-IAP, or ILP2), or variants or mutants thereof, can be assayed in vitro, in vivo, or in a cell line. In vitro assays include assays that determine inhibition of one or more IAPs (e.g., NAIP, cIAP1, cIAP2, XIAP, Survivin, Apollon, ML-IAP, or ILP2), or variants or mutants thereof. Alternate in vitro assays quantitate the ability of the inhibitor to bind to one or more IAPs (e.g., NAIP, cIAP1, cIAP2, XIAP, Survivin, Apollon, ML-IAP, or ILP2), or variants or mutants thereof. Detailed conditions for assaying a compound described herein as an inhibitor of one or more IAPs (e.g., NAIP, cIAP1, cIAP2, XIAP, Survivin, Apollon, ML-IAP, or ILP2), or variants or mutants thereof, are well known in the art and set forth in the Examples below.

The provided compounds are inhibitors of one or more IAPs (e.g., NAIP, cIAP1, cIAP2, XIAP, Survivin, Apollon, ML-IAP, or ILP2), or variants or mutants thereof, and are therefore useful for treating one or more disorders associated with activity of one or more IAPs (e.g., NAIP, cIAP1, cIAP2, XIAP, Survivin, Apollon, ML-IAP, or ILP2). Thus, in some aspects and embodiments, the present disclosure provides a method for treating an IAP-mediated disease, disorder, or condition comprising the step of administering to a patient in need thereof a compound of the present disclosure, or pharmaceutically acceptable composition thereof.

In some embodiments, the present disclosure provides a method of inhibiting one or more IAPs (e.g., NAIP, cIAP1, cIAP2, XIAP, Survivin, Apollon, ML-IAP, or ILP2), or variants or mutants thereof, comprising contacting a cell with a provided compound.

In some embodiments, inhibitors of IAPs include those of the formulae described herein, or a pharmaceutically acceptable salt thereof, wherein each variable is as defined and described herein. In some embodiments, the present disclosure provides a compound of formula I:

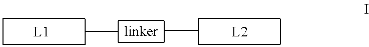

or a pharmaceutically acceptable salt thereof, wherein: L1 is a first ligand; L2 is a second ligand; and linker is a bivalent linker comprising

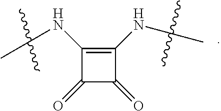 [SMILES: *Nc1c(N*)c(=O)c1=O]

In some embodiments, the present disclosure provides a compound of formula I:

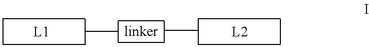

or a pharmaceutically acceptable salt thereof, wherein: L1 is a first ligand; L2 is a second ligand; and linker is a bivalent linker comprising

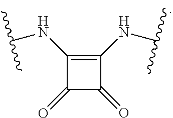 [SMILES: *Nc1c(N*)c(=O)c1=O]

Without wishing to be bound by any particular theory, it is believed that L1 and L2 need to be positioned at a certain distance relative to each other to achieve optimum biological activity. In some embodiments, L1 and L2 need to be positioned at a distance of about 0.5-2.5 nm as measured from the atom on each of L1 and L2 to which the linker is attached. Further, without wishing to be bound by any particular theory, it is believed that such positioning of L1 and L2 relative to each other cannot be achieved with rigid linear linkers. For example, AZD5582 comprises a diyne linker having the structure

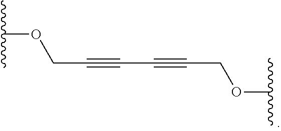 [SMILES: *OCC#CC#CCO*]

See Hennessy et al., J. Med. Chem. 2013, 56, 9897-9919. Hennessy et al. report that the linker should have minimal steric requirements to prevent disruption of critical binding interactions with the target protein. Hennessy et al. further report that a fully saturated linker

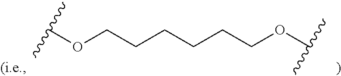 [SMILES: *OCCCCCCO*]

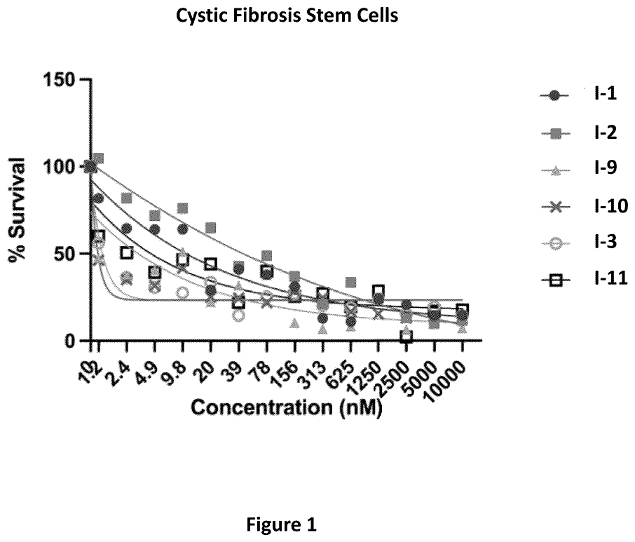

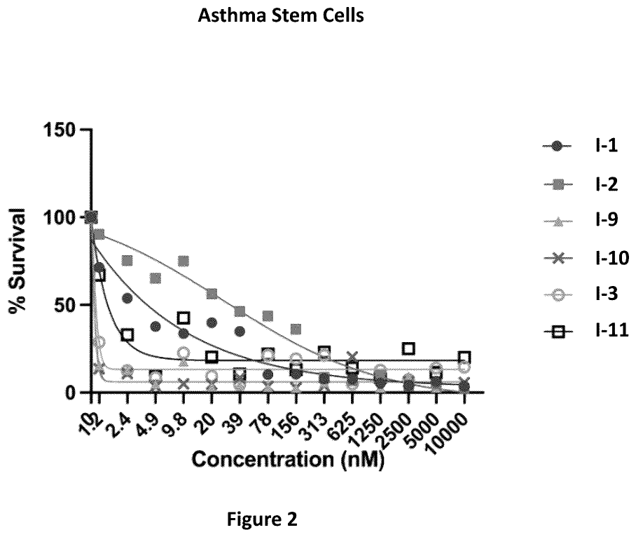

did not result in any appreciable change in the cellular potency relative to compounds such as AZD5582. Hennessy et al. further surmise that shorter, less hydrophobic linkers render compounds less cell-permeable and therefore less potent in cell-based assays. In some embodiments, the present disclosure provides the insight that, despite the teachings of Hennessy, compounds comprising less rigid, more hydrophilic linkers, such as those described herein (e.g., compounds having a linker comprising squaramide) demonstrate improved activity as compared to compounds with rigid, hydrophobic linkers such as AZD5582. See, for example, FIGS. 1 and 2. Additionally, FIGS. 1 and 2 demonstrate that compounds of formula I are more potent than compounds having flexible, hydrophobic linkers such as SM-164 and BV6.

In some embodiments, it will be appreciated that compounds comprising less hydrophobic linkers have lower log Ps relative to compounds comprising more hydrophobic linkers. For example, the log P for AZD5582 is calculated to be 6.14, whereas the log P of compound I-1 is calculated to be 5.5.

Thus, the present disclosure encompasses the insight that compounds of formula I are uniquely potent against cancer cell lines due to the flexibility and hydrophilicity of squaramide linkers as described herein.

As generally defined above, L1 is a first ligand; and L2 is a second ligand. In some embodiments, L1 and L2 are the same. In some embodiments, L1 and L2 are different.

In some embodiments, a ligand (e.g., L1 or L2) refers to a moiety that binds to a protein, for example, at a ligand binding domain. In some embodiments, a ligand (e.g., L1 or L2) is a moiety that binds to an IAP. In some embodiments, an IAP is selected from NAIP, cIAP1, cIAP2, XIAP, Survivin, Apollon, MIL-IAP and ILP2.

In some embodiments, L1 is or comprises a group selected from

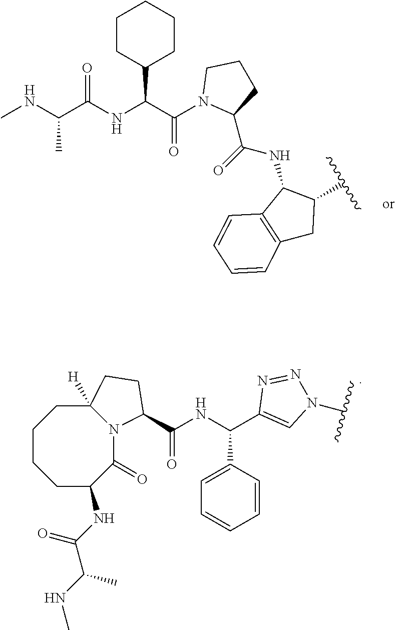 [SMILES: *[C@@H]1Cc2ccccc2[C@@H]1NC(=O)[C@@H]1CCCN1C(=O)[C@@H](NC(=O)[C@H](C)NC)C1CCCCC1 ; *n1cc([C@@H](NC(=O)[C@@H]2CC[C@@H]3CCCC[C@H](NC(=O)[C@H](C)NC)C(=O)N32)c2ccccc2)nn1]

In some embodiments, L2 is or comprises a group selected from

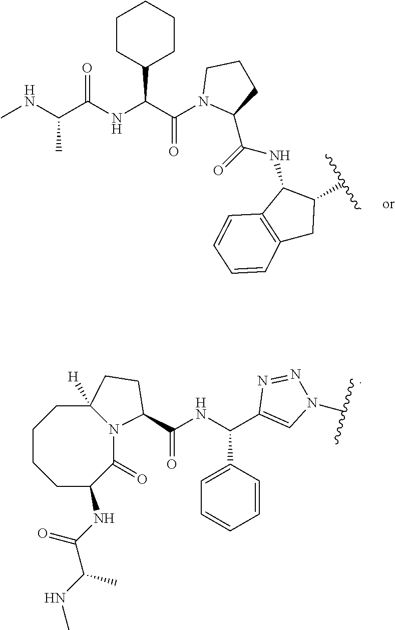 [SMILES: *[C@@H]1Cc2ccccc2[C@@H]1NC(=O)[C@@H]1CCCN1C(=O)[C@@H](NC(=O)[C@H](C)NC)C1CCCCC1 ; *n1cc([C@@H](NC(=O)[C@@H]2CC[C@@H]3CCCC[C@H](NC(=O)[C@H](C)NC)C(=O)N32)c2ccccc2)nn1]

As generally defined above, linker is a bivalent linker comprising

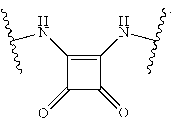 [SMILES: *Nc1c(N*)c(=O)c1=O]

In some embodiments, the linker is of formula X:

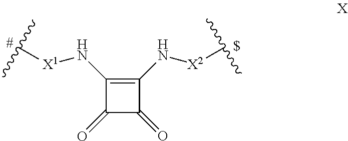

or a pharmaceutically acceptable salt thereof, wherein:
each of X¹ and X² is independently a covalent bond or an optionally substituted bivalent, saturated or partially unsaturated, straight or branched C₁₋₁₂ hydrocarbon chain, wherein 1-4 carbon atoms are optionally and independently replaced by —O—, —N(R)—, —C(O)—, —S—, —SO—, —SO₂—, or -Cy-; each R is independently selected from hydrogen or an optionally substituted C₁₋₆ aliphatic; each -Cy- is independently an optionally substituted bivalent ring selected from a 3- to 8-membered carbocyclene, a 5- to 6-membered saturated or partially unsaturated heterocyclene having 1-3 heteroatoms independently selected from oxygen, nitrogen, or sulfur; phenylene; or a 5- to 6-membered heteroarylene having 1-3 heteroatoms independently selected from oxygen, nitrogen, or sulfur; #represents the point of attachment to L1; and $ represents the point of attachment to L2.

As generally defined above, each of X¹ and X² is independently a covalent bond or an optionally substituted bivalent, saturated or partially unsaturated, straight or branched C₁₋₁₂ hydrocarbon chain, wherein 1-4 carbon atoms are optionally and independently replaced by —O—, —N(R)—, —C(O)—, —S—, —SO—, —SO₂—, or -Cy-. In some embodiments, each of X¹ and X² is independently a covalent bond or an optionally substituted bivalent, saturated or partially unsaturated, straight or branched C₁₋₆ hydrocarbon chain, wherein 1-2 carbon atoms are optionally and independently replaced by —O—, —N(R)—, or —C(O)—. In some embodiments, each of X¹ and X² is independently a covalent bond or an optionally substituted bivalent, saturated or partially unsaturated, straight or branched C₁₋₈ hydrocarbon chain, wherein 1-2 carbon atoms are optionally and independently replaced by —O—, —N(R)—, or —C(O)—. In some embodiments, X¹ and X² are the same. In some embodiments, X¹ and X² are different.

In some embodiments, X¹ is a covalent bond. In some embodiments, X¹ is an optionally substituted bivalent, saturated or partially unsaturated, straight or branched C₁₋₆ hydrocarbon chain, wherein 1-2 carbon atoms are optionally and independently replaced by —O—, —N(R)—, or —C(O)—. In some embodiments, X¹ is an optionally substituted bivalent, saturated or partially unsaturated, straight or branched C₃₋₆ hydrocarbon chain, wherein 1-2 carbon atoms are optionally and independently replaced by —O— or —N(R)—. In some embodiments, X¹ is an optionally substituted bivalent, saturated or partially unsaturated, straight C₃₋₆ hydrocarbon chain, wherein 1-2 carbon atoms are optionally replaced by —O—.

In some embodiments, X¹ is an optionally substituted bivalent, saturated or partially unsaturated, straight C₃ hydrocarbon chain, wherein 1 carbon atom is optionally replaced by —O—. In some embodiments, X¹ is an optionally substituted bivalent, saturated, straight C₃ hydrocarbon chain, wherein 1 carbon atom is optionally replaced by —O—. In some embodiments, X¹ is an optionally substituted bivalent, saturated, straight C₃ hydrocarbon chain, wherein 1 carbon atom is replaced by —O—. In some embodiments, X¹ is an optionally substituted bivalent, saturated, straight C₃ hydrocarbon chain.

In some embodiments, X¹ is an optionally substituted bivalent, saturated or partially unsaturated, straight C₄ hydrocarbon chain, wherein 1 carbon atom is optionally replaced by —O—. In some embodiments, X¹ is an optionally substituted bivalent, saturated, straight C₄ hydrocarbon chain, wherein 1 carbon atom is optionally replaced by —O—. In some embodiments, X¹ is an optionally substituted bivalent, saturated, straight C₄ hydrocarbon chain.

In some embodiments, X¹ is an optionally substituted bivalent, saturated or partially unsaturated, straight C₅ hydrocarbon chain, wherein 1-2 carbon atoms are optionally replaced by —O—. In some embodiments, X¹ is an optionally substituted bivalent, saturated, straight C₅ hydrocarbon chain, wherein 1-2 carbon atoms are optionally replaced by —O—. In some embodiments, X¹ is an optionally substituted bivalent, saturated, straight C₅ hydrocarbon chain, wherein 1 carbon atom is optionally replaced by —O—. In some embodiments, X¹ is an optionally substituted bivalent, saturated, straight C₅ hydrocarbon chain, wherein 1 carbon atom is replaced by —O—. In some embodiments, X¹ is an optionally substituted bivalent, saturated, straight C₅ hydrocarbon chain.

In some embodiments, X¹ is an optionally substituted bivalent, saturated or partially unsaturated, straight C₆ hydrocarbon chain, wherein 1-2 carbon atoms are optionally replaced by —O—. In some embodiments, X¹ is an optionally substituted bivalent, saturated, straight C₆ hydrocarbon chain, wherein 1-2 carbon atoms are optionally replaced by —O—. In some embodiments, X¹ is an optionally substitute bivalent, saturated, straight C₆ hydrocarbon chain, wherein 1 carbon atom is replaced by —O—. In some embodiments, X¹ is an optionally substitute bivalent, saturated, straight C₆ hydrocarbon chain, wherein 2 carbon atoms are replaced by —O—.

In some embodiments, X¹ is an optionally substituted bivalent, saturated or partially unsaturated, straight C₇ hydrocarbon chain, wherein 1-2 carbon atoms are optionally replaced by —O—. In some embodiments, X¹ is an optionally substituted bivalent, saturated, straight C₇ hydrocarbon chain, wherein 1-2 carbon atoms are optionally replaced by —O—. In some embodiments, X¹ is an optionally substitute bivalent, saturated, straight C₇ hydrocarbon chain, wherein 1 carbon atom is replaced by —O—. In some embodiments, X¹ is an optionally substitute bivalent, saturated, straight C₇ hydrocarbon chain, wherein 2 carbon atoms are replaced by —O—.

In some embodiments, X¹ is an optionally substituted bivalent, saturated or partially unsaturated, straight C₈ hydrocarbon chain, wherein 1-2 carbon atoms are optionally replaced by —O—. In some embodiments, X¹ is an optionally substituted bivalent, saturated, straight C₈ hydrocarbon chain, wherein 1-2 carbon atoms are optionally replaced by —O—. In some embodiments, X¹ is an optionally substitute bivalent, saturated, straight C₈ hydrocarbon chain, wherein 1 carbon atom is replaced by —O—. In some embodiments, X¹ is an optionally substitute bivalent, saturated, straight C₈ hydrocarbon chain, wherein 2 carbon atoms are replaced by —O—.

In some embodiments, X¹ is:
covalent bond,

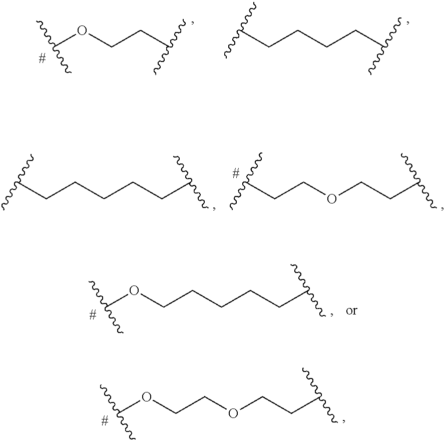 [SMILES: *CCO* ; *CCCC* ; *CCCCC* ; *CCOCC* ; *CCCCCO* ; *CCOCCO*]

wherein #represents the point of attachment to L1.

In some embodiments, X¹ is:
covalent bond,

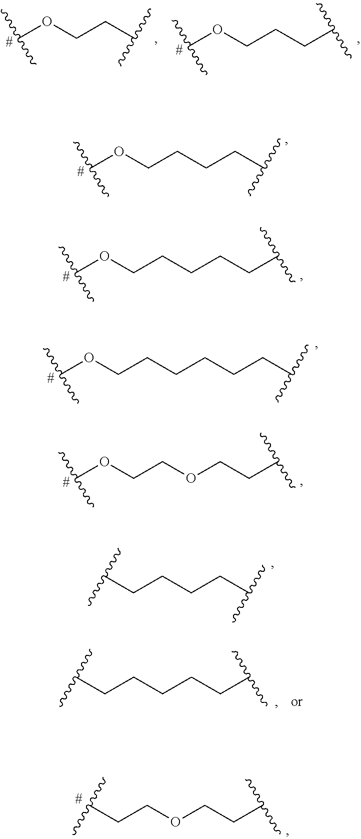

wherein #represents the point of attachment to L1.

In some embodiments, X¹ is

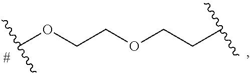 [SMILES: *CCOCCO*]

wherein #represents the point of attachment to L1.

In some embodiments, X¹ is

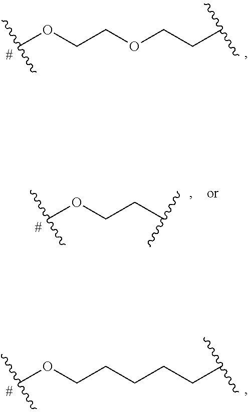 [SMILES: *CCOCCO* ; *CCO* ; *CCCCCO*]

wherein #represents the point of attachment to L1.

In some embodiments, X² is a covalent bond. In some embodiments, X² is an optionally substituted bivalent, saturated or partially unsaturated, straight or branched C₁₋₆ hydrocarbon chain, wherein 1-2 carbon atoms are optionally and independently replaced by —O—, —N(R)—, or —C(O)—. In some embodiments, X² is an optionally substituted bivalent, saturated or partially unsaturated, straight or branched C₃₋₆ hydrocarbon chain, wherein 1-2 carbon atoms are optionally and independently replaced by —O— or —N(R)—. In some embodiments, X² is an optionally substituted bivalent, saturated or partially unsaturated, straight C₃₋₆ hydrocarbon chain, wherein 1-2 carbon atoms are optionally replaced by —O—.

In some embodiments, X² is an optionally substituted bivalent, saturated or partially unsaturated, straight C₃ hydrocarbon chain, wherein 1 carbon atom is optionally replaced by —O—. In some embodiments, X² is an optionally substituted bivalent, saturated, straight C₃ hydrocarbon chain, wherein 1 carbon atom is optionally replaced by —O—. In some embodiments, X² is an optionally substituted bivalent, saturated, straight C₃ hydrocarbon chain, wherein 1 carbon atom is replaced by —O—. In some embodiments, X² is an optionally substituted bivalent, saturated, straight C₃ hydrocarbon chain.

In some embodiments, X² is an optionally substituted bivalent, saturated or partially unsaturated, straight C₄ hydrocarbon chain, wherein 1 carbon atom is optionally replaced by —O—. In some embodiments, X² is an optionally substituted bivalent, saturated, straight C₄ hydrocarbon chain, wherein 1 carbon atom is optionally replaced by —O—. In some embodiments, X² is an optionally substituted bivalent, saturated, straight C₄ hydrocarbon chain.

In some embodiments, X² is an optionally substituted bivalent, saturated or partially unsaturated, straight C₅ hydrocarbon chain, wherein 1-2 carbon atoms are optionally replaced by —O—. In some embodiments, X² is an optionally substituted bivalent, saturated, straight C₅ hydrocarbon chain, wherein 1-2 carbon atoms are optionally replaced by —O—. In some embodiments, X² is an optionally substituted bivalent, saturated, straight C₅ hydrocarbon chain, wherein 1 carbon atom is optionally replaced by —O—. In some embodiments, X² is an optionally substituted bivalent, saturated, straight C₅ hydrocarbon chain, wherein 1 carbon atom is replaced by —O—. In some embodiments, X² is an optionally substituted bivalent, saturated, straight C₅ hydrocarbon chain.

In some embodiments, X² is an optionally substituted bivalent, saturated or partially unsaturated, straight C₆ hydrocarbon chain, wherein 1-2 carbon atoms are optionally replaced by —O—. In some embodiments, X² is an optionally substituted bivalent, saturated, straight C₆ hydrocarbon chain, wherein 1-2 carbon atoms are optionally replaced by —O—. In some embodiments, X² is an optionally substitute bivalent, saturated, straight C₆ hydrocarbon chain, wherein 1 carbon atom is replaced by —O—. In some embodiments, X² is an optionally substitute bivalent, saturated, straight C₆ hydrocarbon chain, wherein 2 carbon atoms are replaced by —O—.

In some embodiments, X² is an optionally substituted bivalent, saturated or partially unsaturated, straight C₇ hydrocarbon chain, wherein 1-2 carbon atoms are optionally replaced by —O—. In some embodiments, X² is an optionally substituted bivalent, saturated, straight C₇ hydrocarbon chain, wherein 1-2 carbon atoms are optionally replaced by —O—. In some embodiments, X² is an optionally substitute bivalent, saturated, straight C₇ hydrocarbon chain, wherein 1 carbon atom is replaced by —O—. In some embodiments, X² is an optionally substitute bivalent, saturated, straight C₇ hydrocarbon chain, wherein 2 carbon atoms are replaced by —O—.

In some embodiments, X² is an optionally substituted bivalent, saturated or partially unsaturated, straight C₈ hydrocarbon chain, wherein 1-2 carbon atoms are optionally replaced by —O—. In some embodiments, X² is an optionally substituted bivalent, saturated, straight C₈ hydrocarbon chain, wherein 1-2 carbon atoms are optionally replaced by —O—. In some embodiments, X² is an optionally substitute bivalent, saturated, straight C₈ hydrocarbon chain, wherein 1 carbon atom is replaced by —O—. In some embodiments, X² is an optionally substitute bivalent, saturated, straight C₈ hydrocarbon chain, wherein 2 carbon atoms are replaced by —O—.

In some embodiments, X² is:
covalent bond,

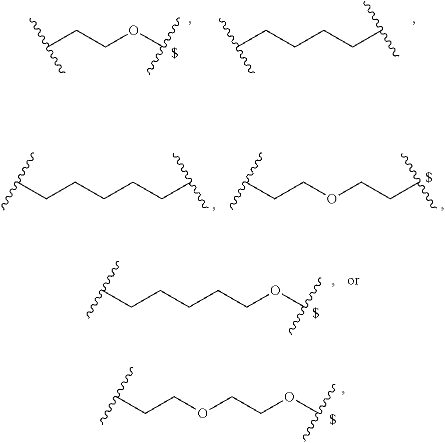 [SMILES: *CCO* ; *CCCC* ; *CCCCC* ; *CCOCC* ; *CCCCCO* ; *CCOCCO*]

wherein $ represents the point of attachment to L2.

In some embodiments, X² is:
covalent bond,

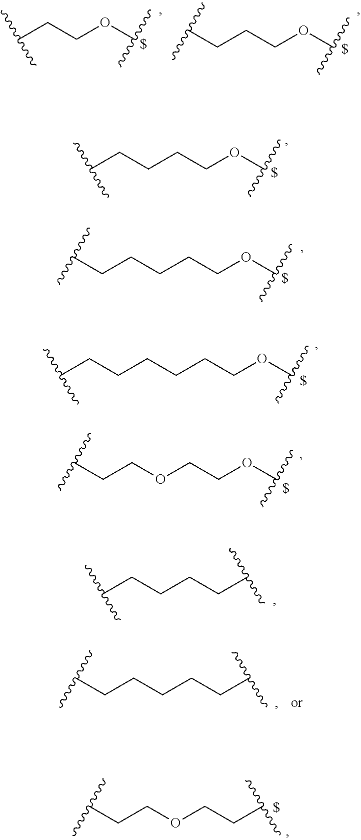

wherein $ represents the point of attachment to L2.

In some embodiments, X² is

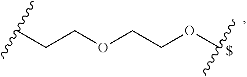 [SMILES: *CCOCCO*]

wherein #represents the point of attachment to L2.

In some embodiments, X² is

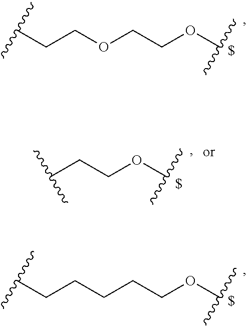 [SMILES: *CCOCCO* ; *CCO* ; *CCCCCO*]

wherein #represents the point of attachment to L2.

As generally defined above, each R is independently selected from hydrogen or an optionally substituted C₁₋₆ aliphatic. In some embodiments R is hydrogen. In some embodiments, R is optionally substituted C₁₋₆ aliphatic.

As generally defined above, each -Cy- is independently an optionally substituted bivalent ring selected from a 3- to 8-membered carbocyclene, a 5- to 6-membered saturated or partially unsaturated heterocyclene having 1-3 heteroatoms independently selected from oxygen, nitrogen, or sulfur; phenylene; or a 5- to 6-membered heteroarylene having 1-3 heteroatoms independently selected from oxygen, nitrogen, or sulfur. In some embodiments, -Cy- is a 3- to 8-membered carbocyclene. In some embodiments, -Cy- is a 5- to 6-membered saturated or partially unsaturated heterocyclene having 1-3 heteroatoms independently selected from oxygen, nitrogen, or sulfur. In some embodiments, -Cy- is phenylene. In some embodiments, -Cy- is a 5- to 6-membered heteroarylene having 1-3 heteroatoms independently selected from oxygen, nitrogen, or sulfur.

In some embodiments, the present disclosure provides a compound of formula I-a, I-b, or I-c:

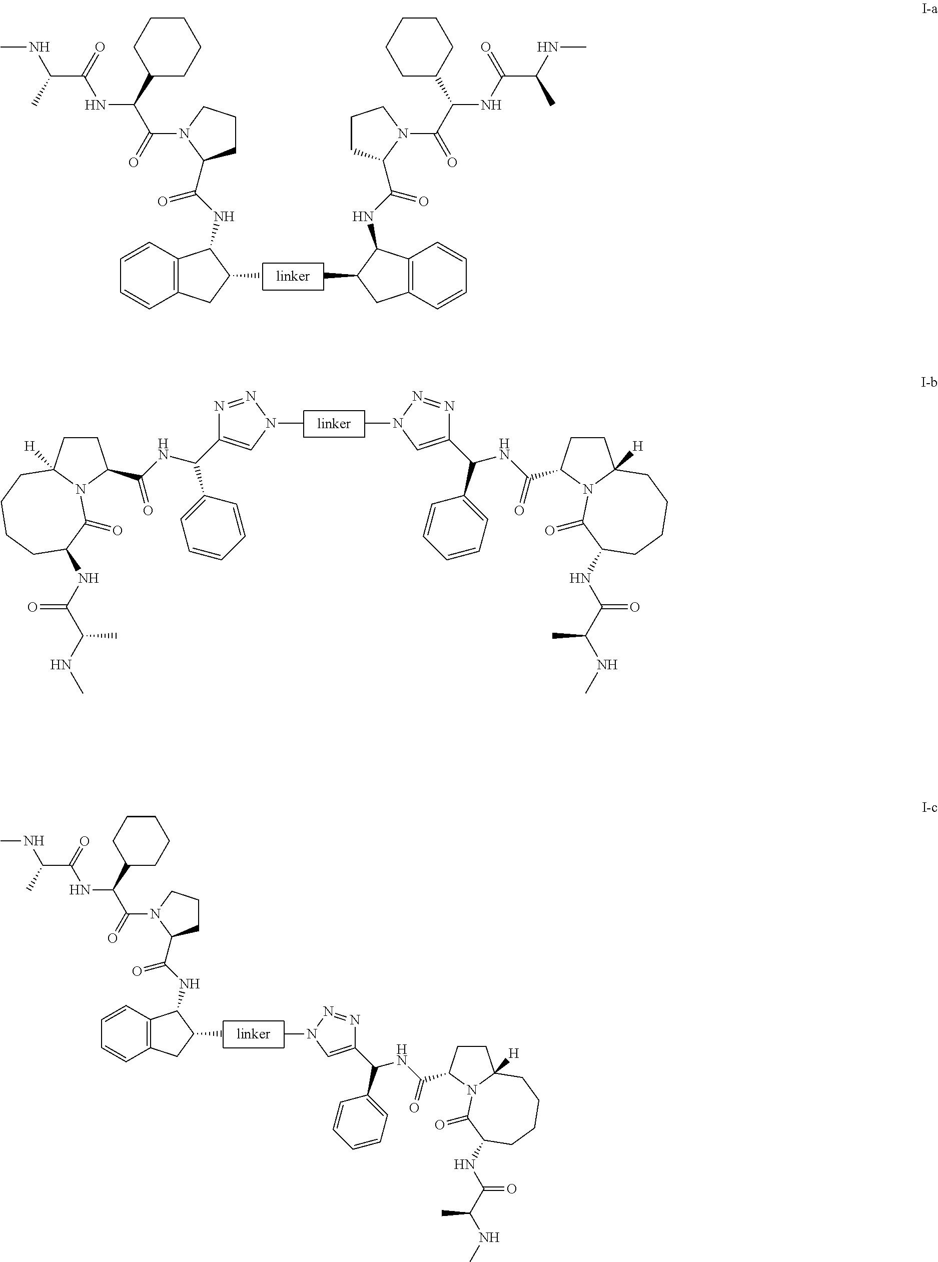

or a pharmaceutically acceptable salt thereof, wherein linker is as defined above and described herein.

In some embodiments, it will be appreciated that the linker serves to position L1 and L2 at a particular distance relative to each other (e.g., between about 0.5-2.5 nm).

In certain embodiments of formula I-a, the linker is sufficient to position L1 and L2 at a distance of about 0.5-2.5 nm between the C1 carbon atoms of the respective indanyl groups (indicated by * below):

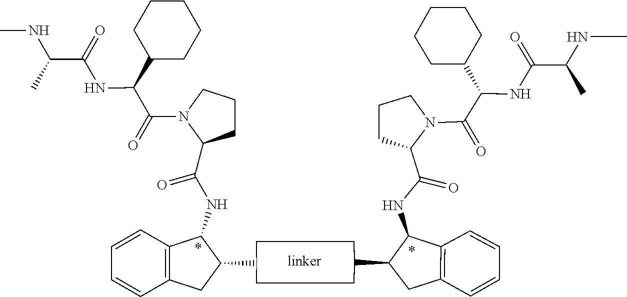

In some embodiments of formula I-a, the linker is sufficient to position L1 and L2 at a distance of about 0.7-2.2 nm between the C1 carbon atoms of the respective indanyl groups. In some embodiments of formula I-a, the linker is sufficient to position L1 and L2 at a distance of about 1.0-2.2 nm between the C1 carbon atoms of the respective indanyl groups. In some embodiments of formula I-a, the linker is sufficient to position L1 and L2 at a distance of about 1.4-2.2 nm between the C1 carbon atoms of the respective indanyl groups. In some embodiments of formula I-a, the linker is sufficient to position L1 and L2 at a distance of about 1.8-2.2 nm between the C1 carbon atoms of the respective indanyl groups. In some embodiments of formula I-a, the linker is sufficient to position L1 and L2 at a distance of about 2.0-2.2 nm between the C1 carbon atoms of the respective indanyl groups. In some embodiments of formula I-a, the linker is sufficient to position L1 and L2 at a distance of about 1.2-1.8 nm between the C1 carbon atoms of the respective indanyl groups. In some embodiments of formula I-a, the linker is sufficient to position L1 and L2 at a distance of about 1.3-1.5 nm between the C1 carbon atoms of the respective indanyl groups. In some embodiments of formula I-a, the linker is sufficient to position L1 and L2 at a distance of about 0.7-1.5 nm between the C1 carbon atoms of the respective indanyl groups. In some embodiments of formula I-a, the linker is sufficient to position L1 and L2 at a distance of about 0.7-1.0 nm between the C1 carbon atoms of the respective indanyl groups. In some embodiments of formula I-a, the linker is sufficient to position L1 and L2 at a distance of about 0.7-0.8 nm between the C1 carbon atoms of the respective indanyl groups. In some embodiments of formula I-a, the linker is sufficient to position L1 and L2 at a distance of about 0.7-0.8, 1.4-1.5, or 2.0-2.2 nm between the C1 carbon atoms of the respective indanyl groups. In some embodiments of formula I-a, the linker is sufficient to position L1 and L2 at a distance of about 0.7, 1.5, or 2.1 nm between the C1 carbon atoms of the respective indanyl groups.

In certain embodiments of formula I-b, the linker is sufficient to position L1 and L2 at a distance of about 1.5-2.5 nm between the respective benzylic carbon atoms (indicated by * below):

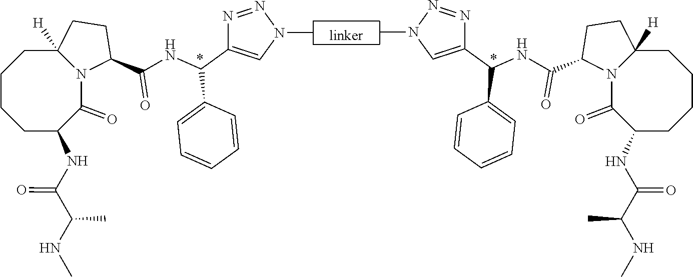

In some embodiments of formula I-b, the linker is sufficient to position L1 and L2 at a distance of about 1.9-2.2 nm between the respective benzylic carbon atoms. In some embodiments of formula I-b, the linker is sufficient to position L1 and L2 at a distance of about 1.9-2.0 nm between the respective benzylic carbon atoms. In some embodiments of formula I-b, the linker is sufficient to position L1 and L2 at a distance of about 2.0-2.2 nm between the respective benzylic carbon atoms. In some embodiments of formula I-b, the linker is sufficient to position L1 and L2 at a distance of about 2.1-2.2 nm between the respective benzylic carbon atoms. In some embodiments of formula I-b, the linker is sufficient to position L1 and L2 at a distance of about 1.9 or 2.1 nm between the respective benzylic carbon atoms.

In certain embodiments of formula I-c, the linker is sufficient to position L1 and L2 at a distance of about 1.5-2.5 nm between the indanyl carbon atom of L1 and the benzylic carbon atom of L2 (indicated by * below):

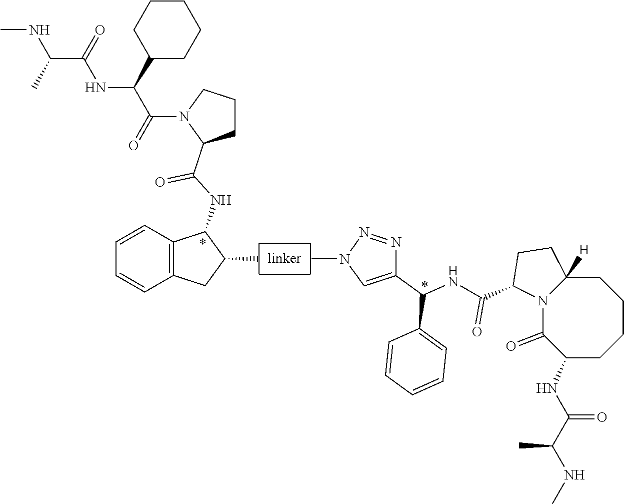

In some embodiments of formula I-c, the linker is sufficient to position L1 and L2 at a distance of about 1.7-2.3 nm between the indanyl carbon atom of L1 and the benzylic carbon atom of L2. In some embodiments of formula I-c, the linker is sufficient to position L1 and L2 at a distance of about 1.9-2.1 nm between the indanyl carbon atom of L1 and the benzylic carbon atom of L2. In some embodiments of formula I-c, the linker is sufficient to position L1 and L2 at a distance of about 1.9-2.1 nm between the indanyl carbon atom of L1 and the benzylic carbon atom of L2.

In some embodiments, a compound of Formula I is selected from:

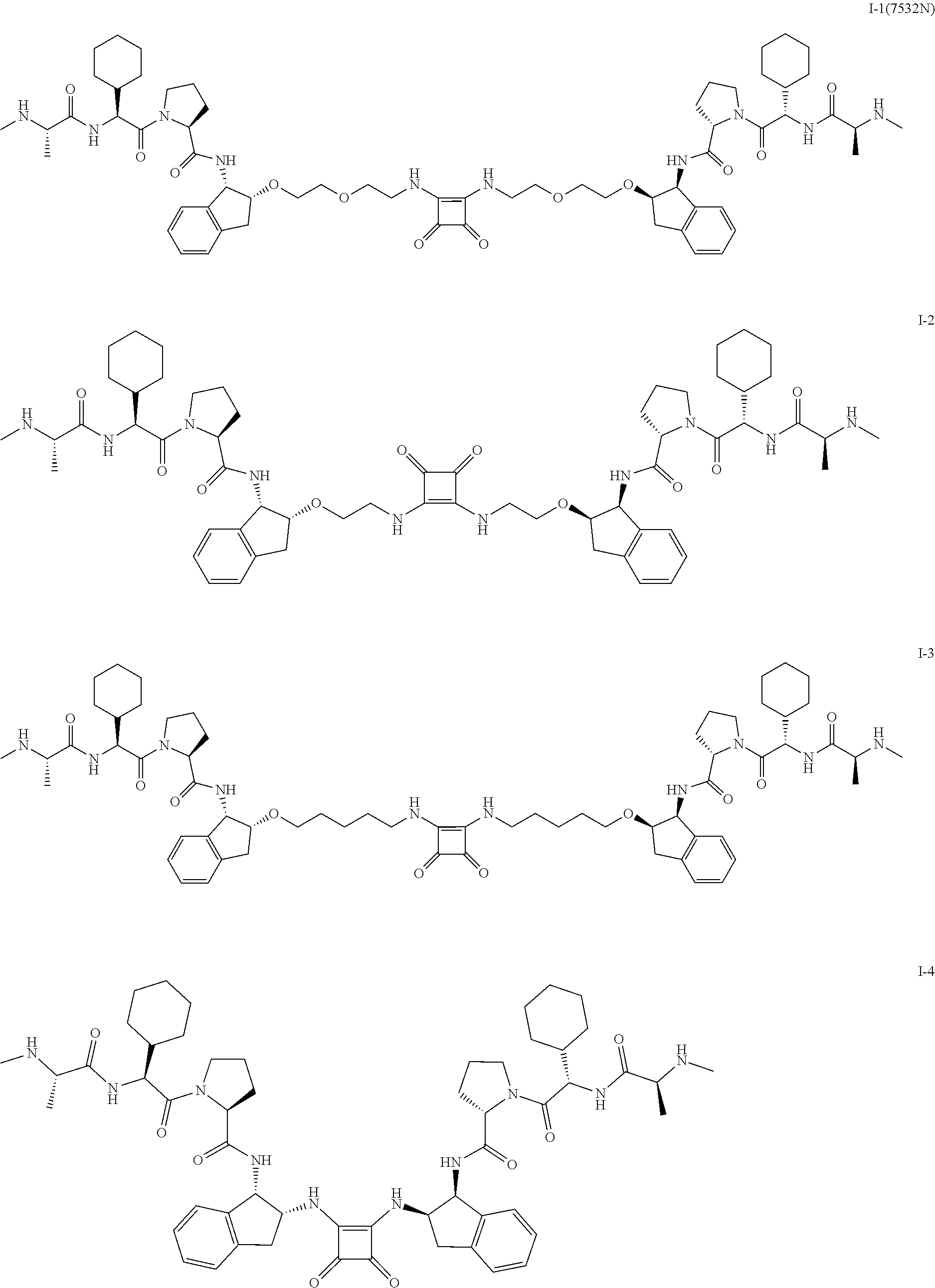

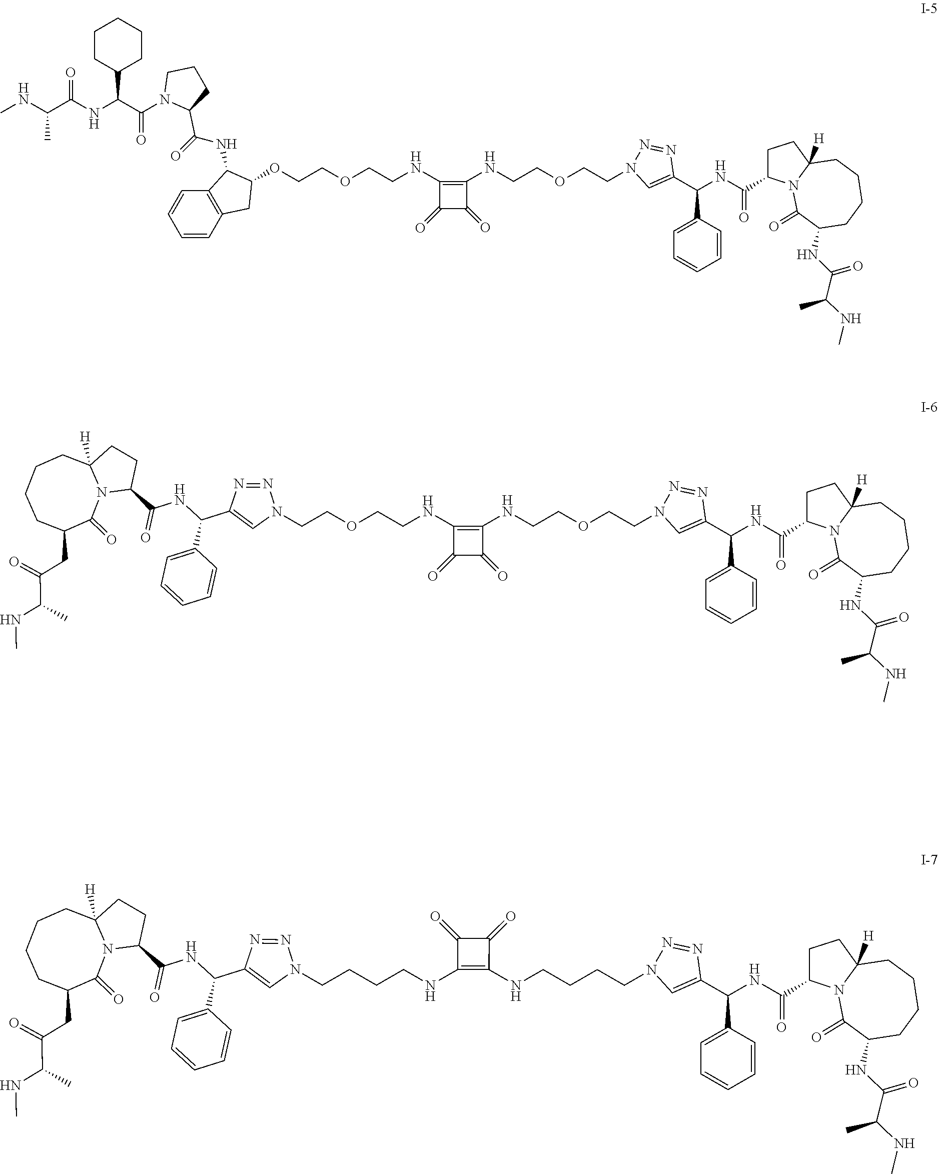

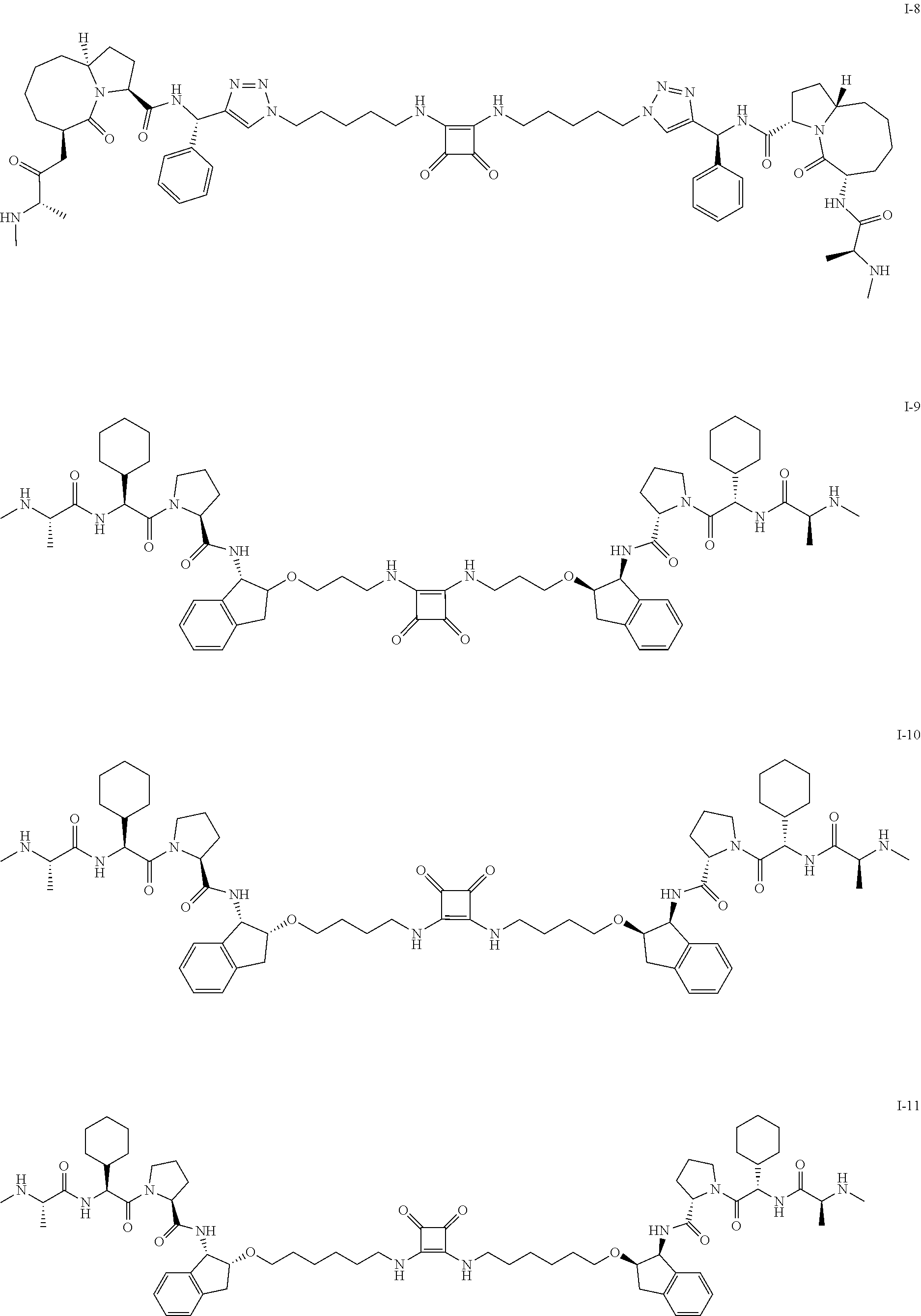

or a pharmaceutically acceptable salt thereof.

| Compound | logP | PSA |
|---|---|---|
| I-1 | 5.49 | 276.20 |
| I-2 | 5.46 | 257.74 |
| I-3 | 7.80 | 257.74 |
| I-4 | 5.42 | 239.28 |
| I-5 | 4.55 | 297.68 |
| I-6 | 3.61 | 319.16 |
| I-7 | 5.14 | 300.70 |
| I-8 | 5.92 | 300.70 |
| I-9 | 6.24 | 257.74 |
| I-10 | 7.02 | 257.74 |
| I-11 | 8.58 | 257.74 |

Compounds of the present disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

Exemplary Methods of Manufacturing Compounds of this Present Disclosure

In some aspects, compounds of the present disclosure may be made by a variety of ways well-known to those skilled in the art of organic synthesis. By way of example, compounds of the present invention can be synthesized using methods described below and/or as described in WO 2007/130626A2 and WO 2010/142994A1, together with synthetic methods known in the art of synthetic organic chemistry, or variations thereon as appreciated by those skilled in the art. Compounds of the present application can be synthesized by the following steps outlined in the General Schemes below. Starting materials are either commercially available or made by known procedures in the reported literature.

In some embodiments, a compound of Formula I-a can be obtained using the method of General Scheme A below:

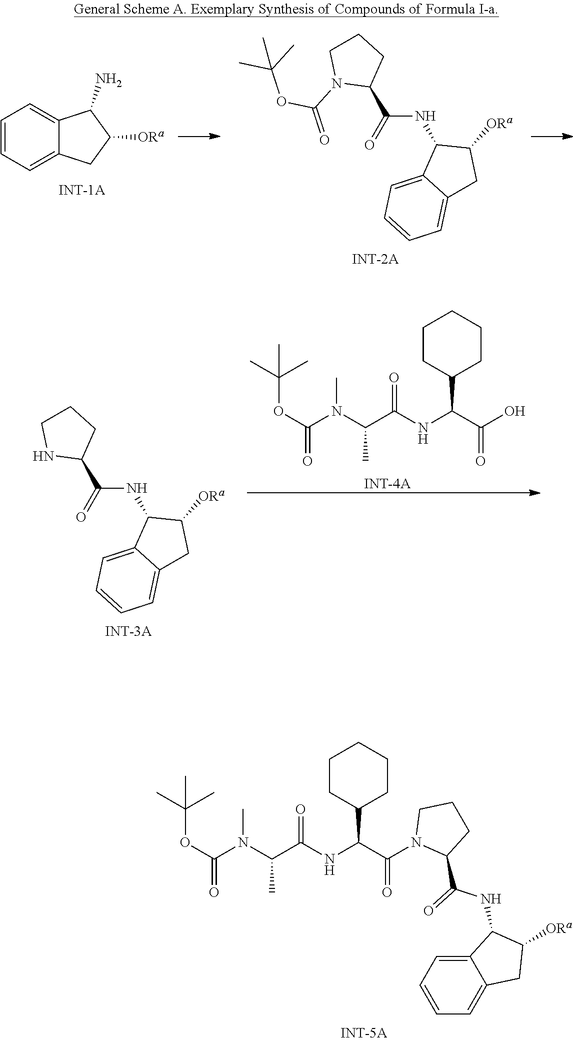

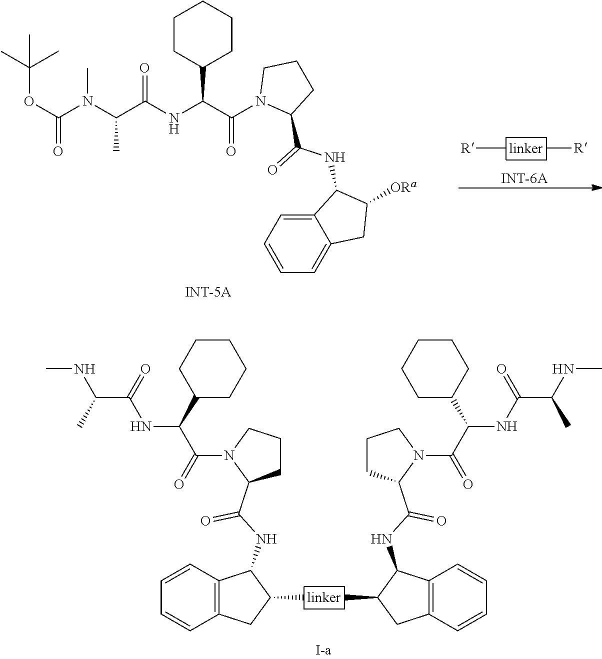

In some embodiments, it will be appreciated that compounds of formula I-a may be obtained as shown in General Scheme A using reagents and reaction conditions well known in the art, e.g., as described in WO 2010/142994A1. In some embodiments, R^{a} is a suitable moiety (or protected analog) where treatment with INT-6A affords a compound of formula I-a. In some embodiments, R′ is a suitable moiety (or protected analog) where treatment with INT-5A affords a compound of formula I-a.

In some embodiments, a compound of Formula I-b can be obtained using the method of General Scheme B below:

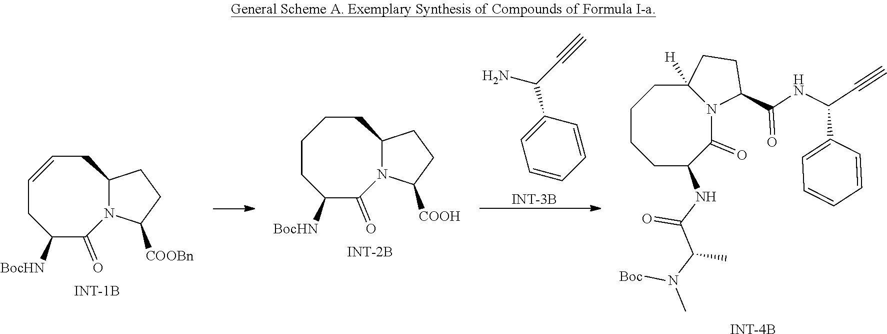

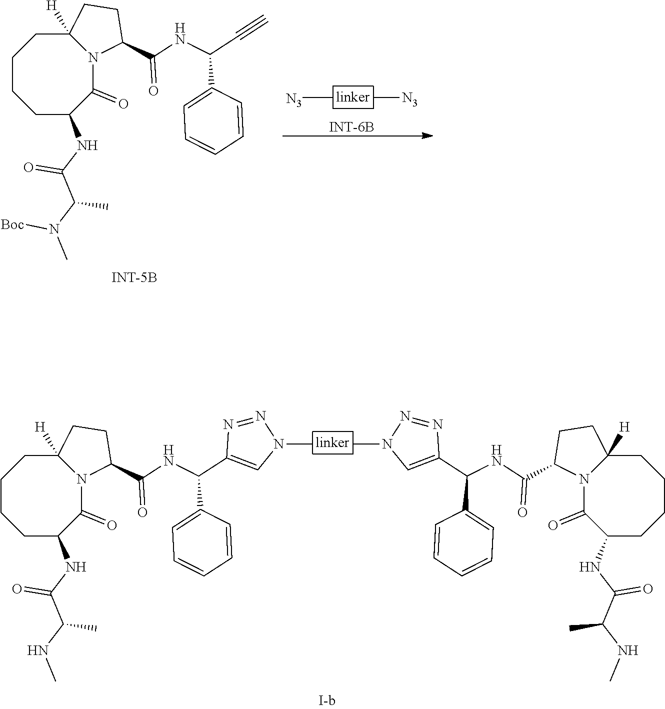

In some embodiments, it will be appreciated that compounds of formula I-b may be obtained as shown in General Scheme B using reagents and reaction conditions well known in the art, e.g., as described in WO 2007/130626A2.

In some embodiments, a compound of Formula I-c can be obtained using the method of General Scheme C below:

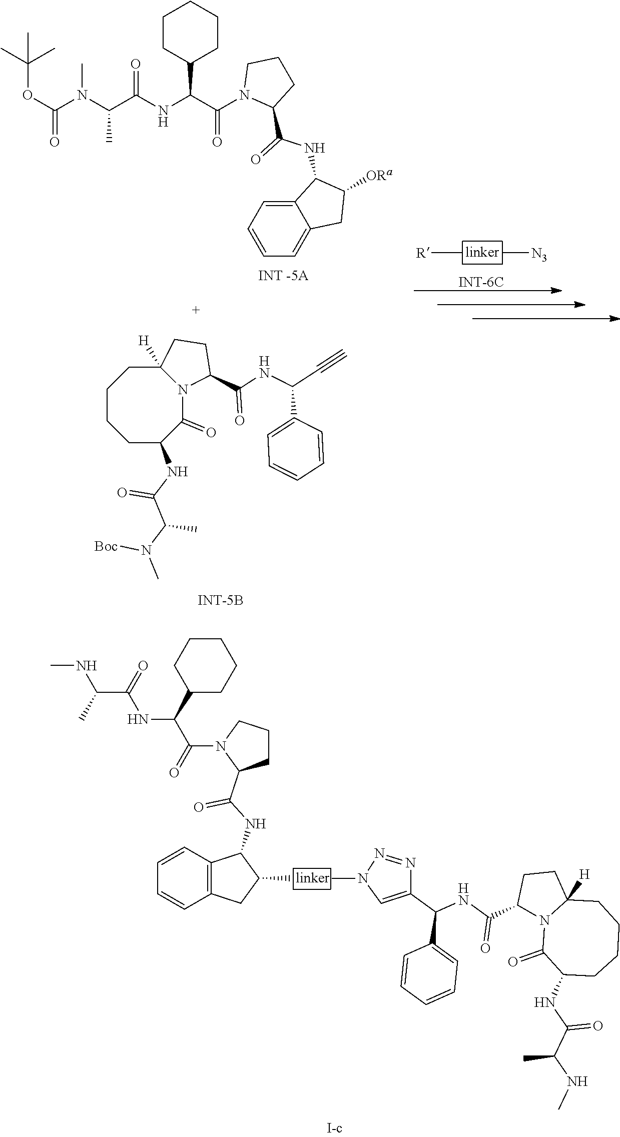

In some embodiments, it will be appreciated that compounds of formula I-c may be obtained as shown in General Scheme C using reagents and reaction conditions well known in the art, e.g., as described in WO 2007/130626A2 and WO 2010/142994A1. In some embodiments, R^{a} is a suitable moiety (or protected analog) where treatment with INT-6C affords a compound of formula I-c. In some embodiments, R′ is a suitable moiety (or protected analog) where treatment with INT-5A affords a compound of formula I-c.

### 2. Inhibitors of Cellular Kinases

In some embodiments, the present disclosure recognizes the importance of inclusion of inhibitors of kinases in treatment of various diseases. In some embodiments the present disclosure recognizes the efficacy of combining inhibitors of kinases with additional compounds in treatment of proliferative disease or disorder. In some embodiments the present disclosure recognizes the particular efficacy of combining inhibitors of kinases with IAP inhibitors as described herein.

In some embodiments an inhibitor of a kinase targets a cellular kinase. One of skill in the art will understand various categories of cellular kinases based on a target of phosphorylation. In some embodiments, a cellular kinase is a Serine/Threonine Protein Kinase (STPKs), a Tyrosine Kinase (TKs), or a Dual Specificity Protein Kinase (DSPKs). One of skill in the art will understand that TKs includes both receptor associated and non-receptor associated tyrosine kinases.

In some embodiments, the present disclosure provides treatments for a proliferative disease or disorder comprising tyrosine kinase inhibitors (TKIs). In some embodiments, a substrate or target of a TKI is one or more of epidermal growth factor receptor (EGFR), ALK (anaplastic lymphoma kinase), TRK (tropomyosin receptor kinase), HER2 (human epidermal growth factor receptor), VEGFR (vascular endothelial growth factor receptor), RET, (Rearranged During Transfection), MET/HGFR (mesenchymal-epithelial transition factor/hepatocyte growth factor receptor), MEK (MAPK/ERK Kinase), FGFR (fibroblast growth factor receptor 1), KIT, PGDFR (platelet-derived growth factor receptor), JAK (Janus kinase), BCR-ABL, SRC, FAK (focal adhesion kinase). In some embodiments, a TKI inhibits the activity of one or more of epidermal growth factor receptor (EGFR), ALK (anaplastic lymphoma kinase), TRK (tropomyosin receptor kinase), HER2 (human epidermal growth factor receptor), VEGFR (vascular endothelial growth factor receptor), RET, (Rearranged During Transfection), MET/HGFR (hepatocyte growth factor receptor), MEK (MAPK/ERK Kinase), FGFR (fibroblast growth factor receptor 1), KIT, PGDFR (platelet-derived growth factor receptor), JAK (Janus kinase), TRK (tropomyosin receptor kinase) BCR-ABL, SRC, FAK (focal adhesion kinase).

In some embodiments, a treatment for a proliferative disease or disorder comprises a TKI. In some embodiments, a TKI is crizotinib, cabozantinib, ponatinib, nintedanib, lestaurtinib, altiratinib, foretinib, merestinib, osimertinib, almonertinib, furmonertinib (AST2818), lazertinib (YH25448), BPI-7711, nazartinib (EGF816), brigatinib, poziotinib, ceritinib, lorlatinib, repotrectinib, lapatinib, neratinib, pyrotinib, tucatinib, donatinib, sorafenib, sunitinib, pazopanib, axitinib, apatinib, vandetanib, selpercatinib, pralsetinib, tepotinib, camaptinib, savolitinib, tepotinib, camaptinib, savolitinib, ibrutinib, acalabrutinib, or zanubrutinib. In some embodiments, a TKI is ponatinib.

### 3. Proliferative Diseases or Disorders

In some embodiments, the present disclosure provides methods and compositions useful for the treatment for various proliferative diseases or disorders. In some embodiments, a proliferative disease or disorder is one defined by excessive proliferation or infiltration of cells. In some embodiments, a proliferative disease or disorder is one defined by metaplasia or dysplasia. In some embodiments, the present disclosure provides methods and compositions useful for the treatment of proliferative diseases or disorders responsive to induction of apoptotic cell death, e.g., disorders characterized by dysregulation of apoptosis.

In some embodiments, a proliferative disease or disorder contemplated by the present disclosure is any disease or disorder associated with, caused by, or resulting from disease associated stem cells. In some embodiments, a disease associated stem cell is a stem cell that exhibits pro-inflammatory and/or pro-fibrotic features. In some embodiments, the present disclosure provides methods and compositions useful for eliminating or inhibiting the growth of disease associated stem cells. In some embodiments, a proliferative disease or disorder is any disease or disorder associated with aberrant growth of a disease associated stem cell derived from epithelial tissue.

In some embodiments, a proliferative disease or disorder contemplated by the present disclosure is a cancer. In some embodiments, a proliferative disease or disorder contemplated by the present disclosure is a pulmonary disease or disorder.

In some embodiments, the present disclosure provides compositions and method for treating, ameliorating, or lessening the severity of a cancer. In some embodiments, a method for treating, ameliorating, or lessening the severity of a cancer comprises administering to a subject in need thereof, a compound or combination as described herein, or a pharmaceutical salt or composition thereof. In some embodiments a cancer is any aberrant or uncontrolled growth of an epithelial tissue. In some embodiments, a cancer is breast cancer, prostate cancer, lung cancer, lymphoma, skin cancer, pancreatic cancer, colon cancer, melanoma, malignant melanoma, ovarian cancer, brain cancer, primary brain carcinoma, head-neck cancer, glioma, glioblastoma, medulloblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, gastrointestinal cancer, rectal cancer, cholangiocarcinoma (bile duct cancer) thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma.

In some embodiments, the present disclosure provides compositions and methods for treating, ameliorating, or lessening the severity of a pulmonary disease, disorder, or condition. In some embodiments, a method for treating, ameliorating, or lessening the severity of a pulmonary disease, disorder, or condition comprises administering to a subject in need thereof, a compound or combination as described herein, or a pharmaceutical salt or composition thereof. In some embodiments, a pulmonary disease comprises an inflammatory disease or condition. In some embodiments, a pulmonary disease is a disease associated with a stem cell that exhibits pro-inflammatory and/or pro-fibrotic features. In some embodiments, a pulmonary disease, disorder, or condition is chronic obstructive pulmonary disease (COPD), cystic fibrosis, airway inflammation, allergy(ies), asthma, impeded respiration, Acute respiratory distress syndrome, pulmonary hypertension, lung inflammation, bronchitis, airway obstruction, bronchoconstriction, microbial infection, viral infection (such as SARS), idiopathic pulmonary fibrosis, bronchopulmonary dysplasia (BPD), chronic bronchitis or emphysema, interstitial lung diseases, COVID interstitial lung disease, or COVID-19.

### 4. Uses, Formulation, and Administration:

In some embodiments, the present disclosure provides a composition comprising a compound described herein or a pharmaceutically acceptable salt thereof and a pharmaceutically acceptable carrier, adjuvant, or vehicle. In some embodiments, the amount of compound in compositions described herein is such that it is effective to measurably inhibit activity of an IAP (e.g., BIRC1/NAIP, BIRC2/cIAP1, BIRC3/cIAP2, BIRC4/XIAP, BIRC5/Survivin, BIRC6/Apollon, BIRC7/ML-IAP and BIRC8/ILP2), or a mutant thereof, in a biological sample or in a patient or subject. In some embodiments, a composition described herein is formulated for administration to a patient in need of such composition. In some embodiments, a composition described herein is formulated for oral administration to a patient.

In some embodiments, the present disclosure provides, compositions, pharmaceutical compositions, preparations, or kits comprising a compound described herein (e.g., an inhibitor of IAP) and/or a cellular kinase inhibitor. In some embodiments, the present disclosure provides, compositions, pharmaceutical compositions, preparations, or kits comprising a compound described herein (e.g., an inhibitor of IAP) and/or a tyrosine kinase inhibitor. In some embodiments, the present disclosure provides, compositions, pharmaceutical compositions, preparations, or kits comprising a compound described herein (e.g., an inhibitor of IAP) and/or ponatinib. In some embodiments, the present disclosure provides, compositions, pharmaceutical compositions, preparations, or kits comprising an inhibitor of IAP selected from I-1, I-2, I-3, I-4, I-5, I-6, I-7, I-8, I-9, I-10, or I-11 and ponatinib.

In some embodiments, compositions, pharmaceutical compositions, preparations, or kits of the present disclosure comprise therapeutically effective amounts of a compound described herein (e.g., an inhibitor of IAP) and/or a cellular kinase inhibitor. In some embodiments, components or agents of the present disclosure (e.g., an inhibitor of IAP and/or a cellular kinase inhibitor) are not mixed together in the same composition. For example, the two agents are not part of the same solution or powder. In some embodiments, the agents or components are kept separate in different compositions and are delivered separately. In some embodiments, a kit may contain a pharmaceutical composition of a compound described herein (e.g., an inhibitor of IAP) and pharmaceutical composition of a cellular kinase inhibitor. In some embodiments, given synergistic interactions between inhibitors of IAP and cellular kinase inhibitors as described herein the amount of one or both agents is lower than the amount that is typically administered when the agent is administered alone. In certain embodiments, the amount of both agents is lower.

In some embodiments, compositions, pharmaceutical compositions, preparations, or kits of the present disclosure may include other agents. In some embodiments, the other agents may be any other therapeutic agent that would be useful to administer to the subject. In certain embodiments, the invention provides for the administration of a compound described herein (e.g., an inhibitor of IAP) and a cellular kinase inhibitor in combination with one or more other therapeutic agents, e.g., another cytotoxic agent, steroidal agent, analgesic, etc. In certain embodiments, the other therapeutic agent is another chemotherapeutic agent. In certain embodiments, the other therapeutic agent is a steroidal agent (e.g., prednisone, dexamethasone, prednisolone). The other therapeutic agent may include an agent for alleviating or reducing any side effects of a compound described herein (e.g., an inhibitor of IAP) and/or a cellular kinase inhibitor. In some embodiments, the other therapeutic agent is an anti-inflammatory agent such as aspirin, ibuprofen, acetaminophen, etc., pain reliever, anti-nausea medication, or anti-pyretic. In certain embodiments, the other therapeutic agent is an agent to treat gastrointestinal disturbances such as nausea, vomiting, stomach upset, and diarrhea. These additional agents may include anti-emetics, anti-diarrheals, fluid replacement, electrolyte replacement, etc. In some embodiments, the other therapeutic agent is an electrolyte replacement or supplementation such as potassium, magnesium, and calcium, in particular, potassium and magnesium. In some embodiments, the other therapeutic agent is an anti-arrhythmic agent. In some embodiments, the other therapeutic agent is a platelet booster, for example, an agent that increases the production and/or release of platelets. In some embodiments, the other therapeutic agent is an agent to boost the production of blood cells such as erythropoietin. In certain embodiments, the other therapeutic agent is an agent to prevent hyperglycemia. In certain embodiments, the other therapeutic agent is an immune system stimulator.

It will also be appreciated that certain of the agents utilized in accordance with the present invention can exist in free form for treatment, or where appropriate, as a pharmaceutically acceptable form thereof. According to the present invention, a pharmaceutically acceptable form includes, but is not limited to, pharmaceutically acceptable salts, esters, salts of such esters, protected forms, stereoisomers, isomers, reduced forms, oxidized forms, tautomers, or any other adduct or derivative which upon administration to a patient in need is capable of providing, directly or indirectly, an agent as otherwise described herein, or a metabolite or residue thereof, e.g., a prodrug.

Compounds and compositions, according to the present disclosure, are administered using any amount and any route of administration effective for treating or lessening the severity of a proliferative disease or disorder provided herein. The exact amount required will vary from subject to subject, depending on the species, age, and general condition of the subject, the severity of the infection, the particular agent or combination of agents, its mode of administration, and the like. Compounds and/or combinations described herein are preferably formulated in unit dosage form for ease of administration and uniformity of dosage.

Compositions of the present disclosure may be administered orally, parenterally, by inhalation spray, topically, rectally, nasally, buccally, vaginally, intraperitoneally, intracisternally or via an implanted reservoir. In some embodiments, the compositions are administered orally, intraperitoneally or intravenously.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

In some embodiments, provided pharmaceutically acceptable compositions are formulated for oral administration. Such formulations may be administered with or without food. In some embodiments, pharmaceutically acceptable compositions described herein are administered without food. In other embodiments, pharmaceutically acceptable compositions described herein are administered with food. Pharmaceutically acceptable compositions described herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and/or i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Alternatively, pharmaceutically acceptable compositions described herein may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Compositions for rectal or vaginal administration are preferably suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Pharmaceutically acceptable compositions described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds described herein include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions described herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. In some embodiments pharmaceutically acceptable compositions described herein can be administered by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the disclosure comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Dosage forms for topical or transdermal administration of a compound disclosed herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### EXEMPLIFICATION

As depicted in the Examples below, in certain exemplary embodiments, compounds are prepared according to the following general procedures. It will be appreciated that, although the general methods depict the synthesis of certain compounds of the present disclosure, the following general methods, and other methods known to one of ordinary skill in the art, can be applied to all compounds and subclasses and species of each of these compounds, as described herein.

### Example 1: Treatment of Pulmonary Disease

As described in Rao et. al. Cell 181, p. 848-864, the entire contents of which is incorporated herein by reference, single-cell cloning technologies applied to biopsies from patients with and without COPD isolated normal distal airway progenitor cells from patients without COPD and variant progenitors epigenetically committed to distinct metaplastic lesions from patients with COPD. The variant progenitor cells induced pathologies (e.g., mucous and squamous metaplasia, neutrophilic inflammation, and fibrosis) as seen in COPD when transplanted into immunodeficient mice. Similar disease variant stem cells were identified in idiopathic pulmonary fibrosis and cystic fibrosis (Wang et al., Sci. Transl Med. 2023 Apr. 26; 15 (693) and Wang et al., Am J. Respir Crit Care Med, 2023 Sep. 11, respectively the entire contents of each of which are incorporated herein by reference in their entirety).

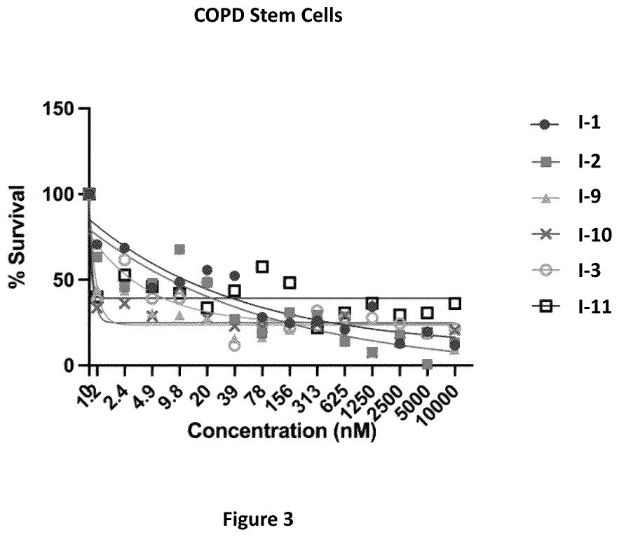

Stem cell variants isolated from subjects suffering from cystic fibrosis were treated with a combination of compounds described herein and cellular kinase inhibitors. Specifically, cystic fibrosis stem cells were treated with ponatinib and compounds described herein. FIG. 1 demonstrates a combination of ponatinib with compounds described herein are efficacious in eliminating cystic fibrosis stem cells. Stem cells isolated from subjects suffering from asthma or COPD were also shown to be sensitive to treatment with a combination of ponatinib and compounds described herein. See FIG. 2 and FIG. 3, respectively.

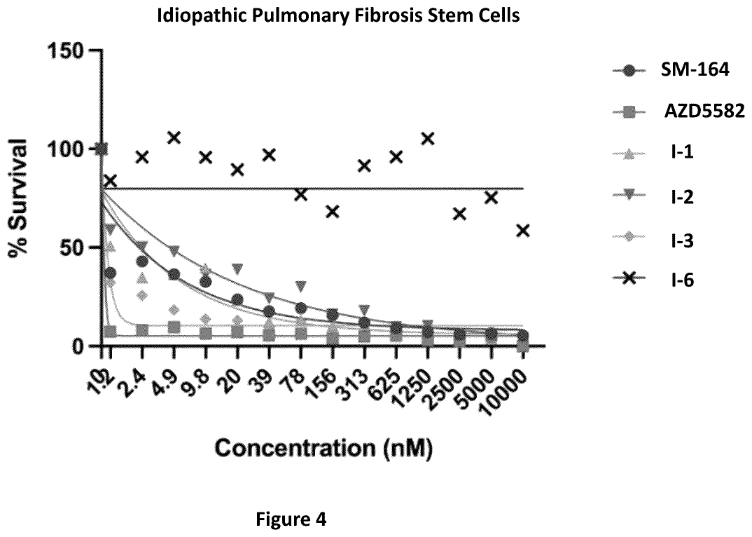

A combination of known IAP inhibitors and ponatinib was compared to a combination of IAP inhibitors as described herein and ponatinib in treatment of stem cells derived from patients suffering from idiopathic pulmonary fibrosis. FIG. 4 demonstrates the efficacy of compounds as described herein in combination with ponatinib in the treatment of idiopathic pulmonary fibrosis.

### Example 2: Treatment of Cancer

The present example demonstrates treatment of various cancers with the compositions described herein.

### Pancreatic Cancer

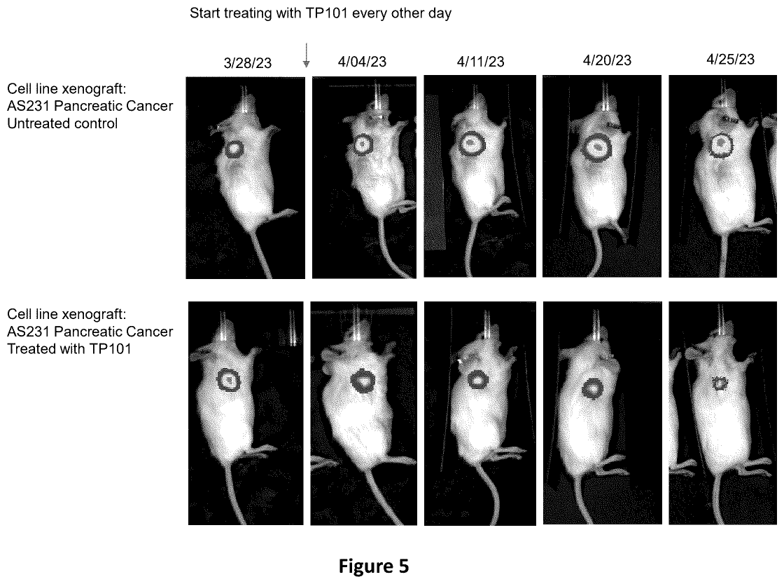

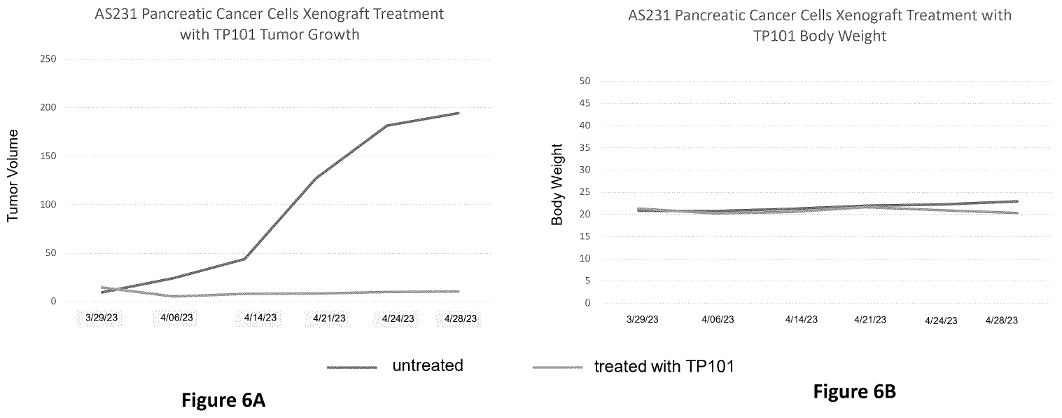

Combination of I-1 as described herein and ponatinib (i.e., TP101) are effective at reducing xenograft tumor burden relative to an untreated control (FIG. 5). Further, FIG. 6A and FIG. 6B quantify the volume of tumors and the weight of the mice xenografted with pancreatic cancer cells.

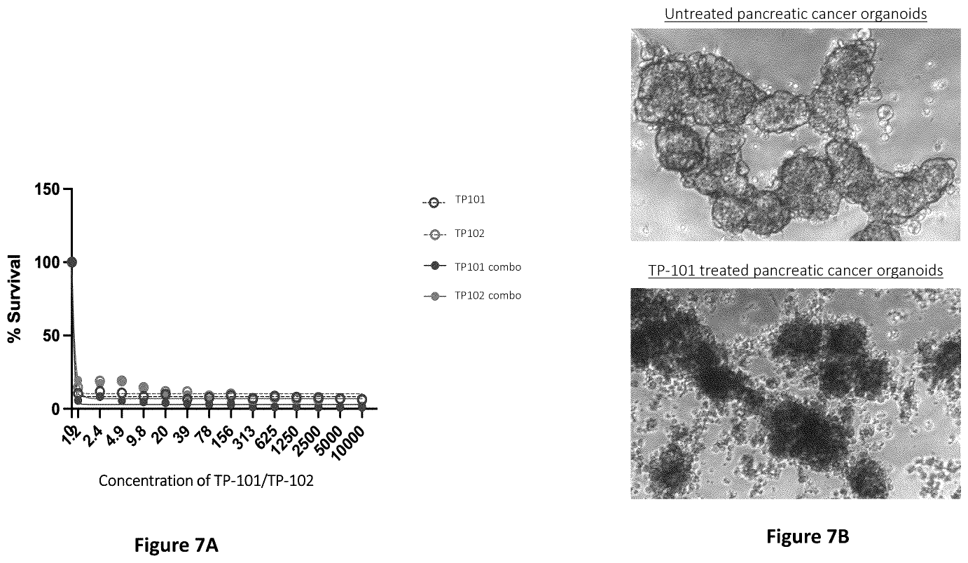

FIGS. 7A and 7B demonstrate the efficacy of TP101 (TP101=combination of I-1 and ponatinib) in treating pancreatic cancer in 2D and 3D cultures of AS231 pancreatic cancer cell line.

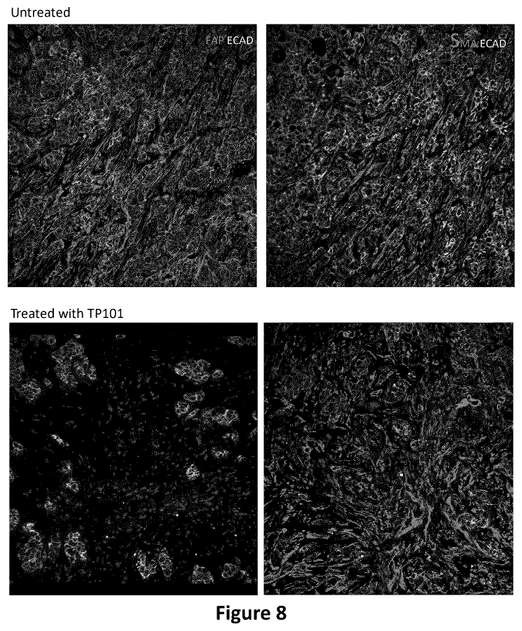

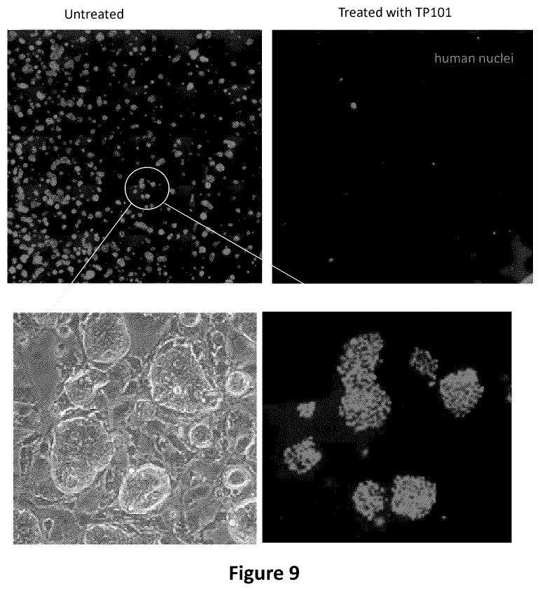

FIG. 8 shows reduction in FAP (red) in pancreatic cells treated with TP101. FIG. 9 shows reduction in the number of clonogenic cancer cells when treated with TP101.

### Esophageal Cancer

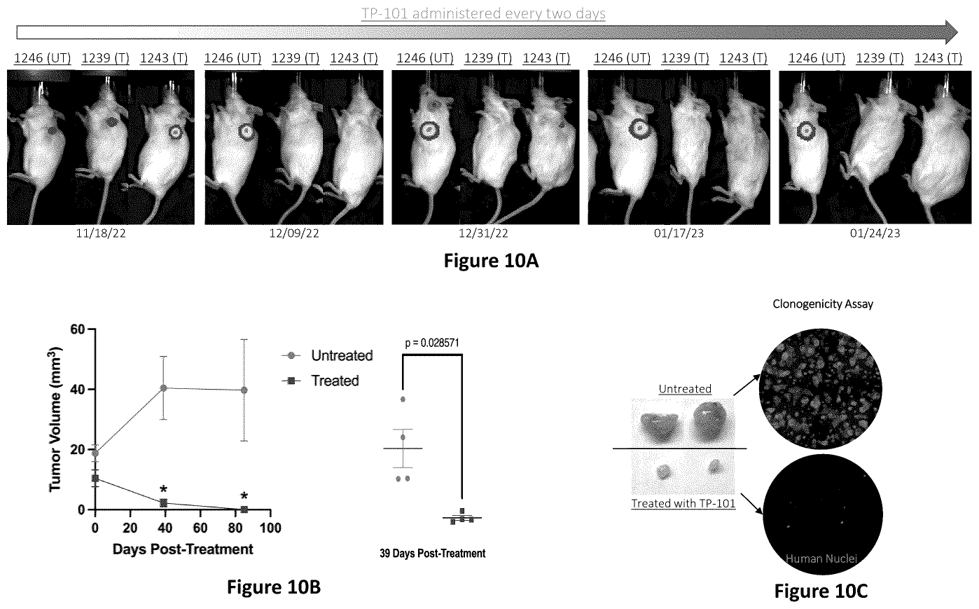

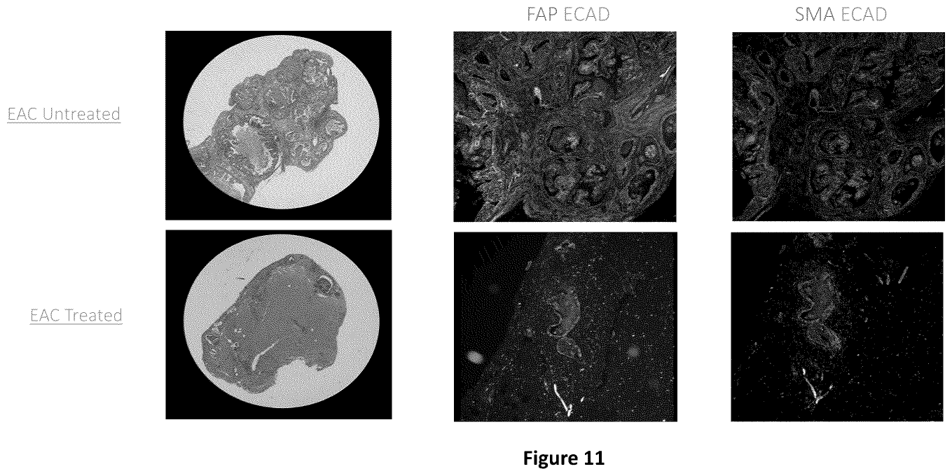

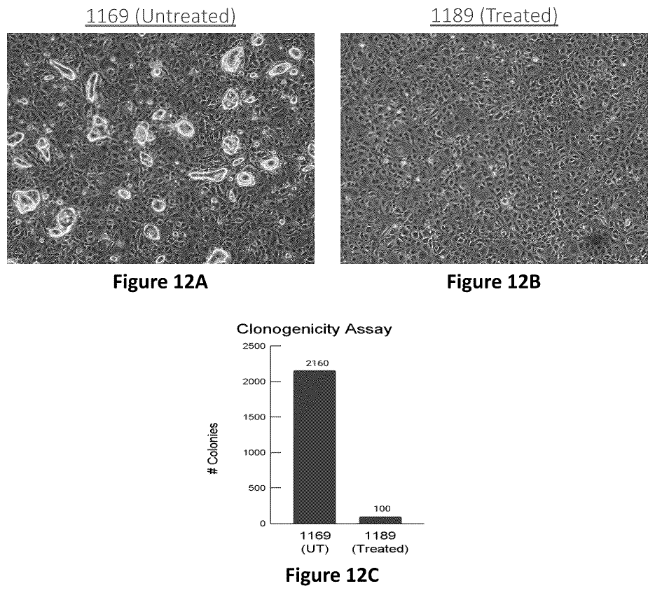

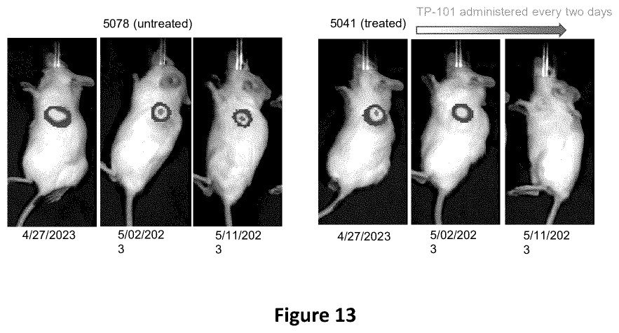

Treatment of xenografted esophageal cancer cells with TP101 (combination of I-1 and ponatinib) results in a decrease in tumor volume (FIGS. 10A; 10B; 10C; UT=untreated; T=treated). FIG. 11 shows reduction in FAP (red) in esophageal adenocarcinoma cells. FIGS. 12A, 12B, and 12C demonstrate that treatment with TP101 reduces cell growth and clonogenicity of cultured esophageal adenocarcinoma cells. Moreover, stem cells from subjects suffering from Barret's Esophagus, often a precursor to esophageal cancer, are particular sensitive to treatment with TP-101 (FIG. 13)

### Gastric Cancer

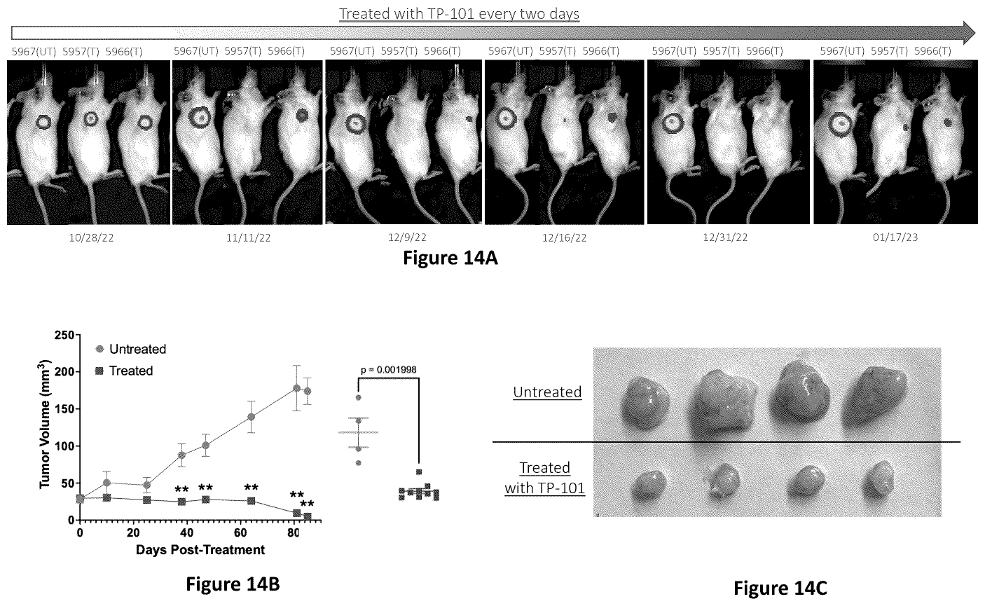

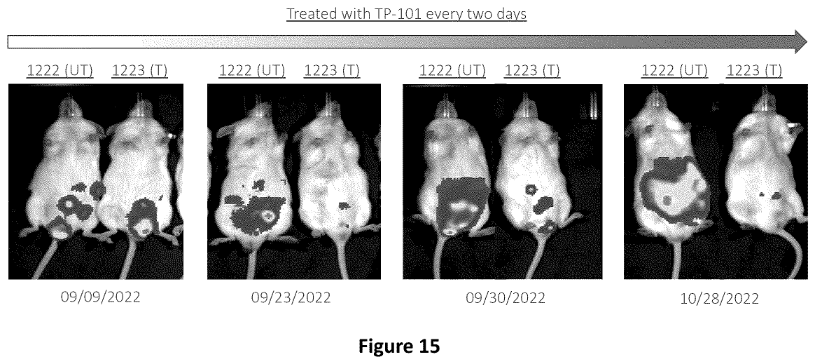

FIGS. 14A; 14B; and 14C demonstrates treatment of xenografted gastric cancer cells with TP101 (combination of I-1 and ponatinib) results in a decrease in tumor volume. FIG. 15 demonstrates the efficacy of TP101 on xenografted gastric cancer cells with ascites.

### Ovarian Cancer

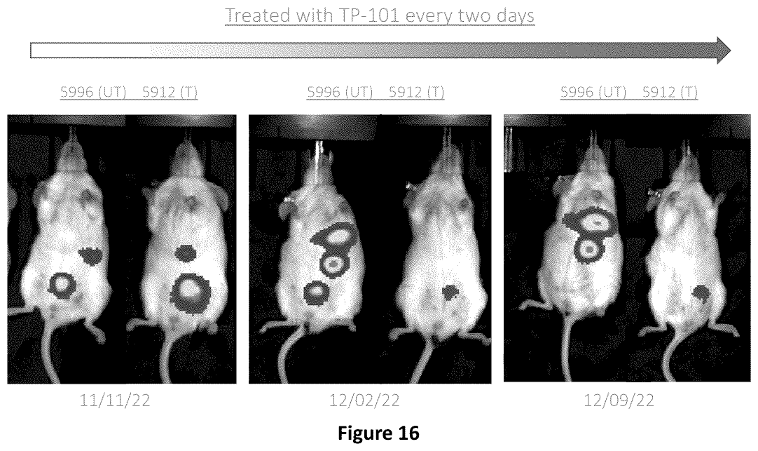

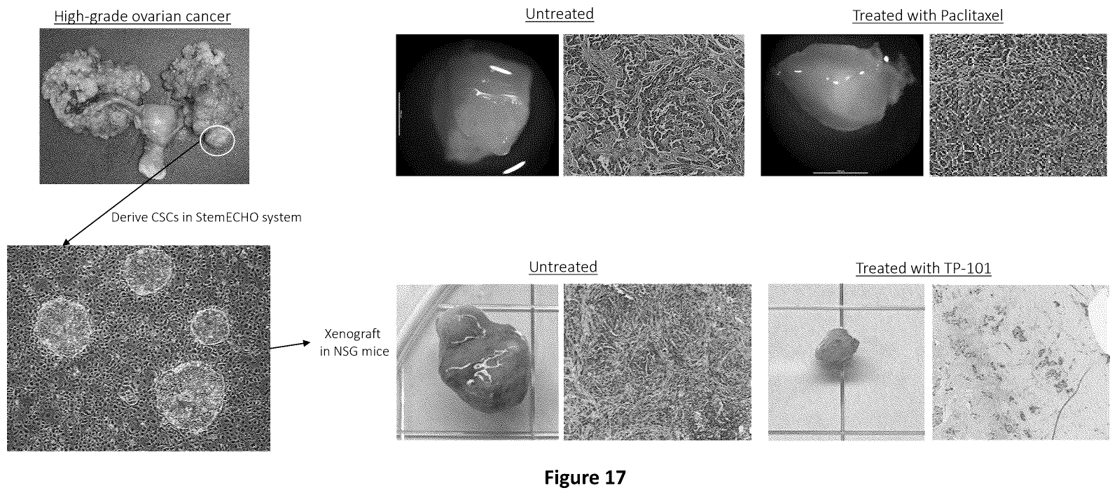

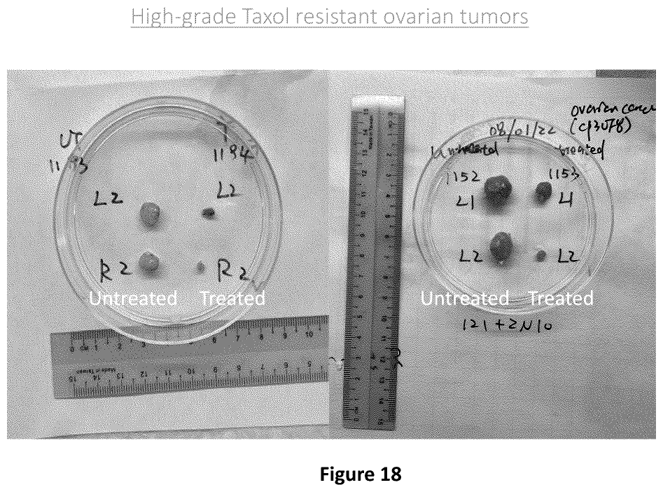

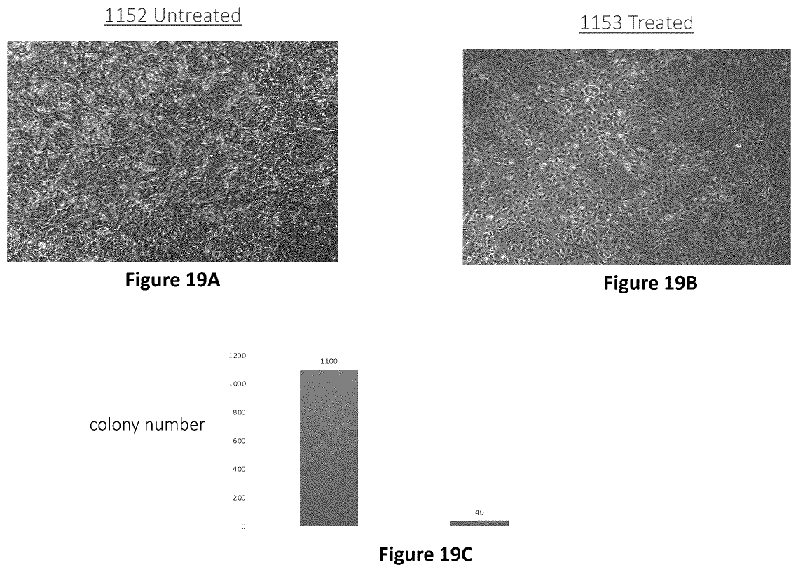

Xenografted ovarian cancer stem cells were sensitive to treatment with TP-101 (FIG. 16). Further, high grade chemotherapy resistant ovarian cancer is sensitive to TP-101 (FIG. 17 and FIG. 18). TP-101 reduces the ability of ovarian tumor cells to replicate and form colonies FIG. 19A; FIG. 19B; FIG. 19C).

### Glioblastoma & Neuroglioma

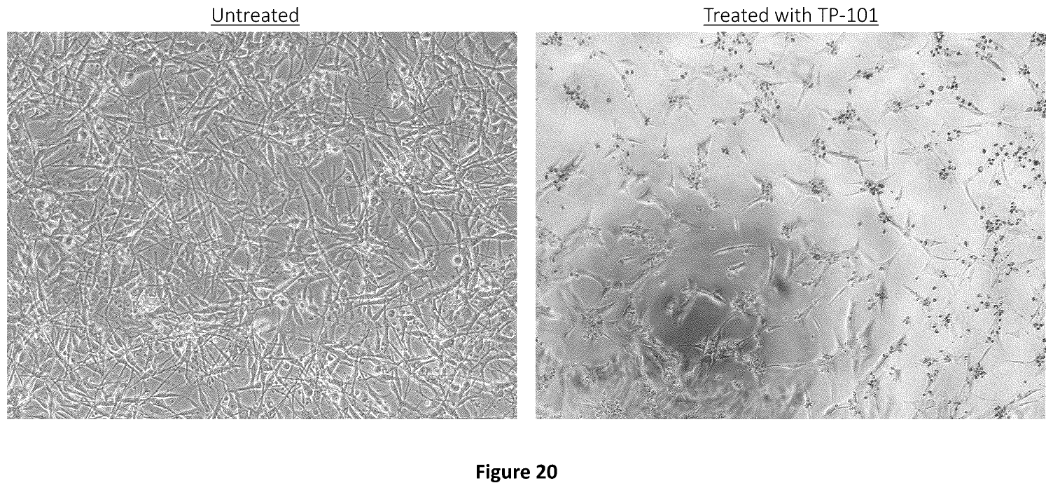

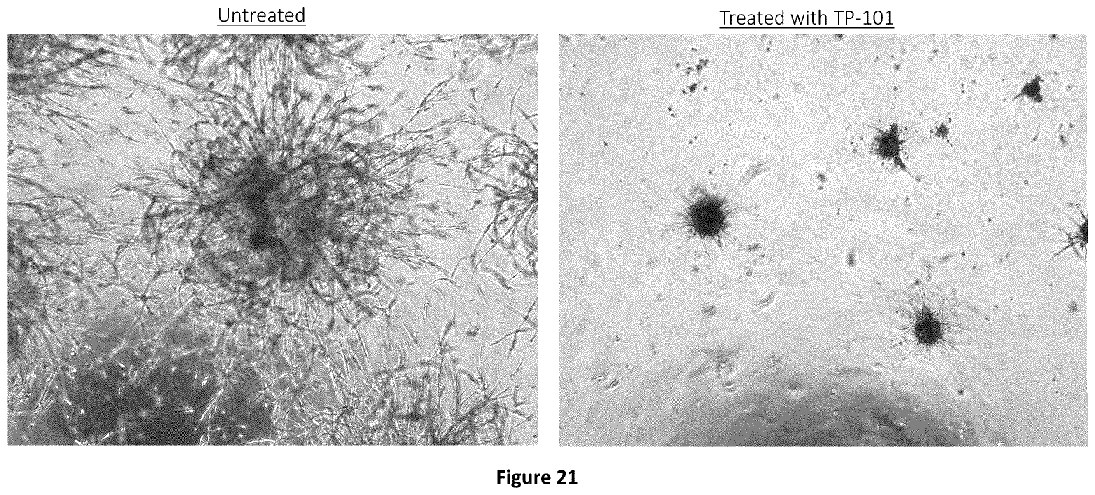

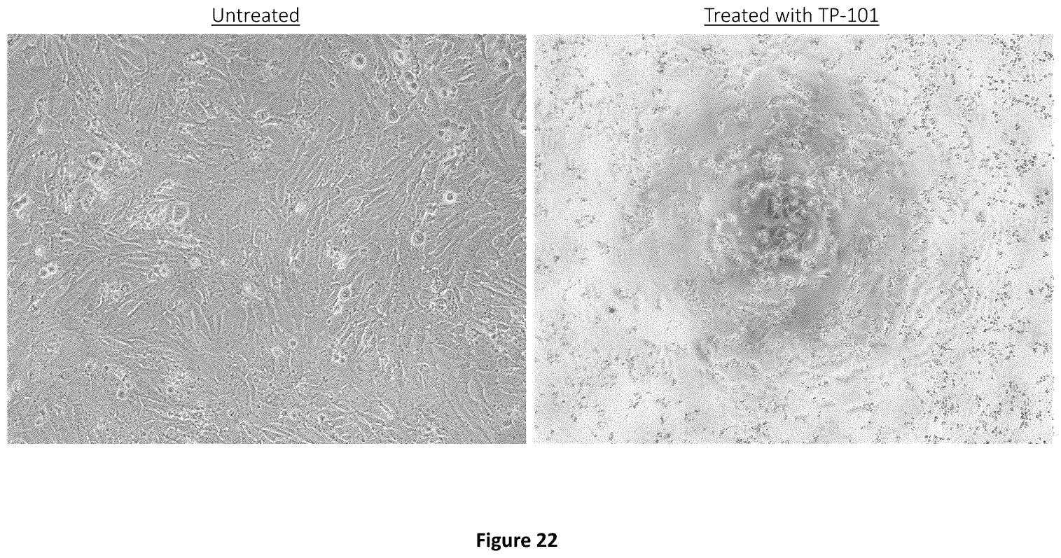

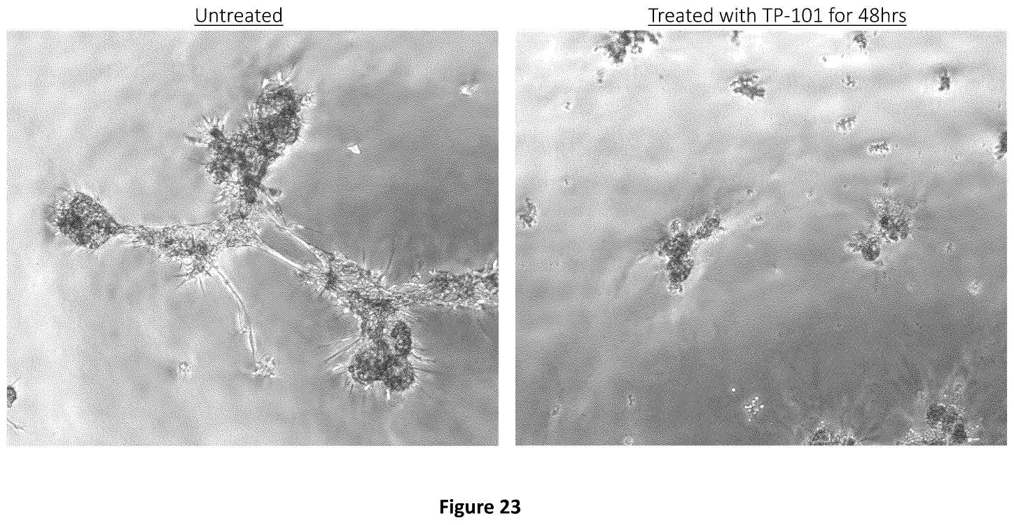

A172 (ATCC: CRL-1620), a cell line isolated from brain tissue of a 53-year-old, male patient with glioblastoma, was sensitive to TP101 in both 2D (FIG. 20) and 3D (FIG. 21) culture. H4 (ATCC: HTB-148), an epithelial cell line isolated from the brain of a 37 year-old, male with neuroglioma. was sensitive to TP101 in both 2D (FIG. 22) and 3D (FIG. 23) culture.

### Lung Cancer

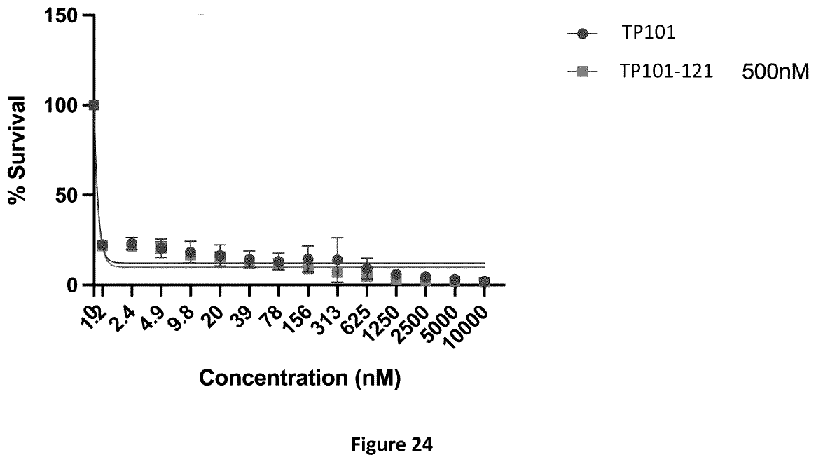

FIG. 24 demonstrates the sensitivity of lung cancer stem cells to TP-101.

### Liver Cancer

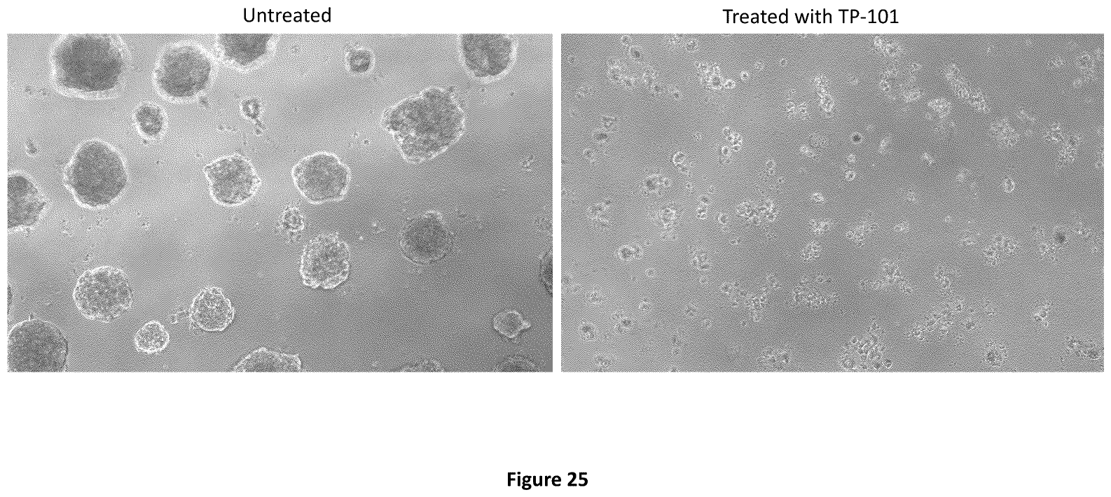

FIG. 25 demonstrates the sensitivity of liver cancer stem cells to TP-101.

### Colon Cancer

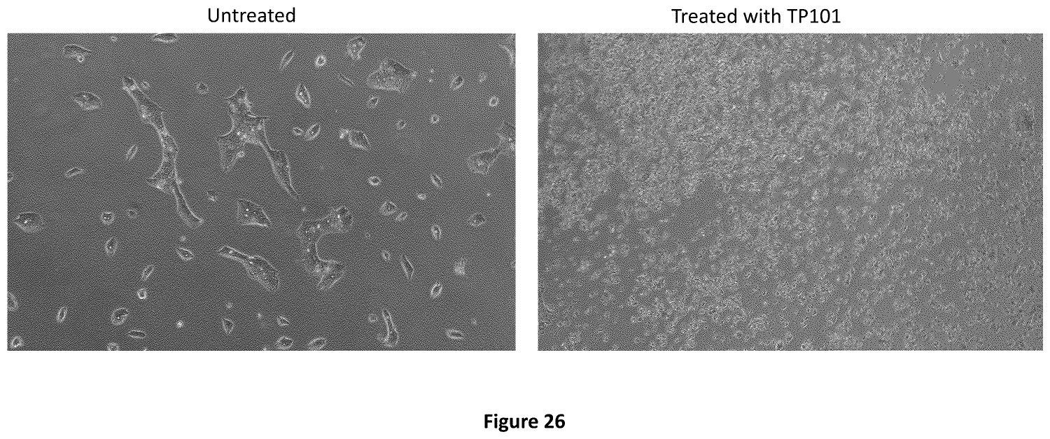

T84 (CCL-248; ATCC), a transplantable human carcinoma cell line isolated from the lung metastasis of a 72-year-old, male, colon cancer patient, was sensitive to TP-101 (FIG. 26).

### Esophageal Cancer

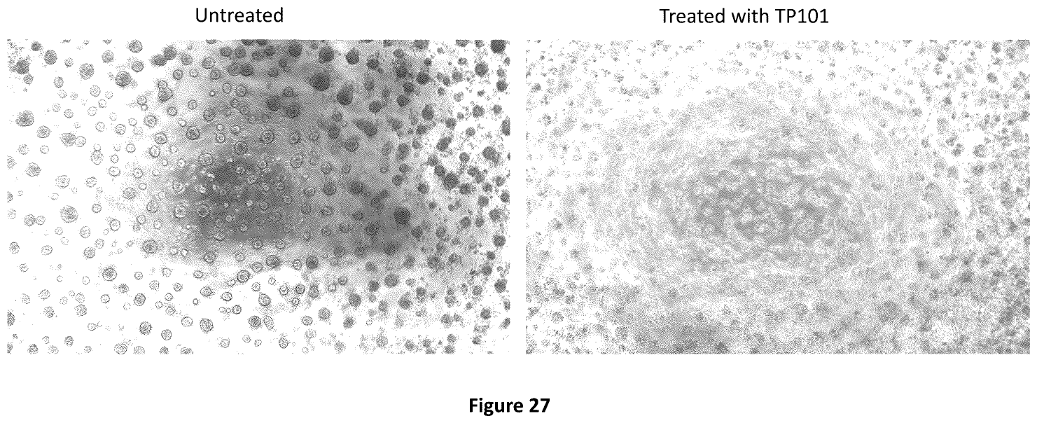

Detroit 562 (CCL138), cells isolated from the pharynx of a pharyngeal cancer patient were sensitive to TP-101 (FIG. 27).

### Breast Cancer

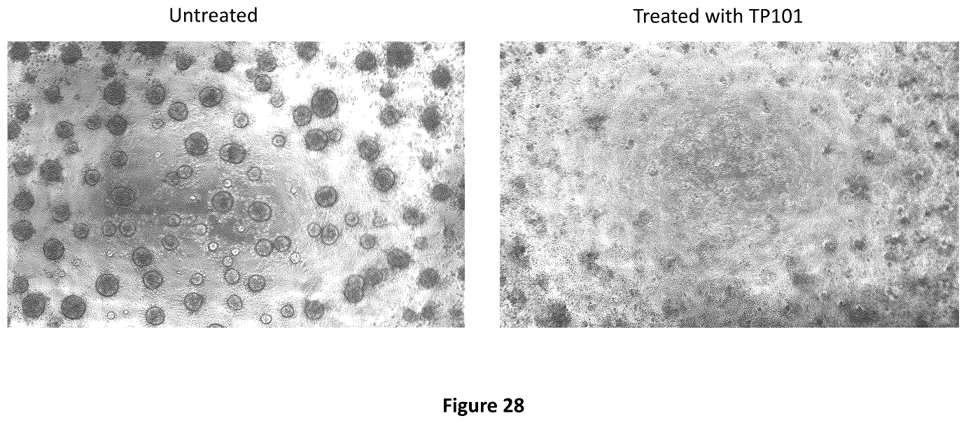

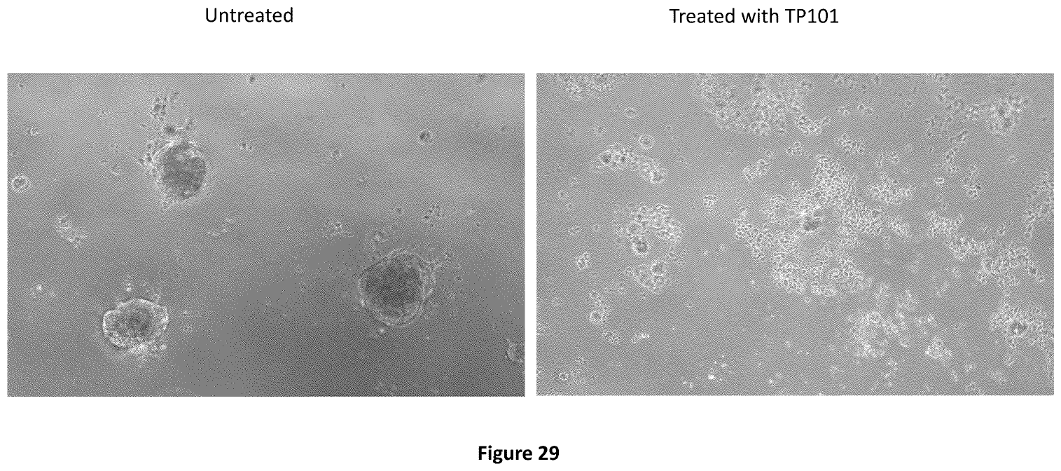

HCC70 (ATCC CRL-2315™), an epithelial cell line isolated from a primary ductal carcinoma is positive for the epithelial cell specific marker Epithelial Glycoprotein 2 (EGP2) and for cytokeratin 19, HCC38, epithelial cells isolated from a mammary gland of a subject with a prior history of leiomyosarcoma, were sensitive to TP-101 (FIGS. 28 and 29, respectively)

### Example 3: Compositions Described Herein are Non-Toxic

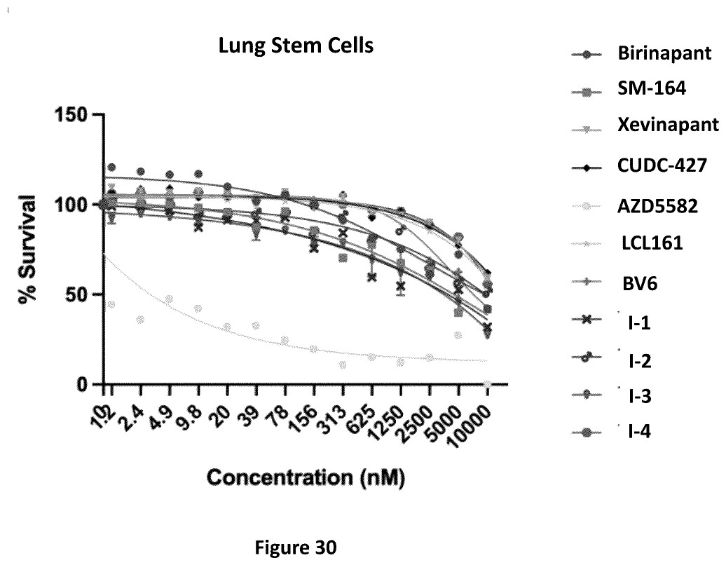

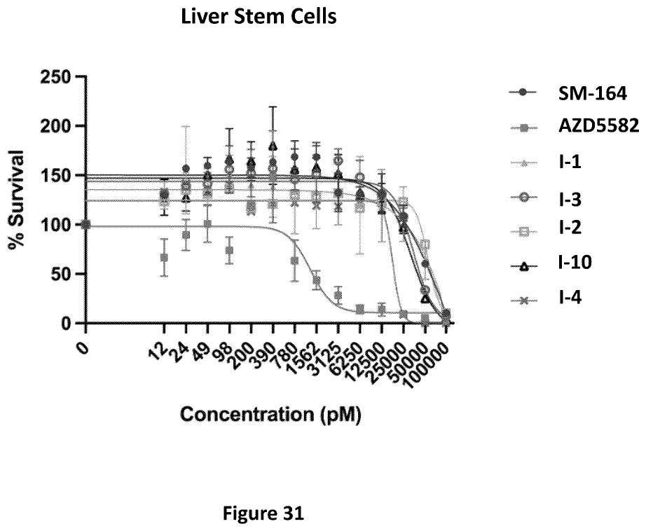

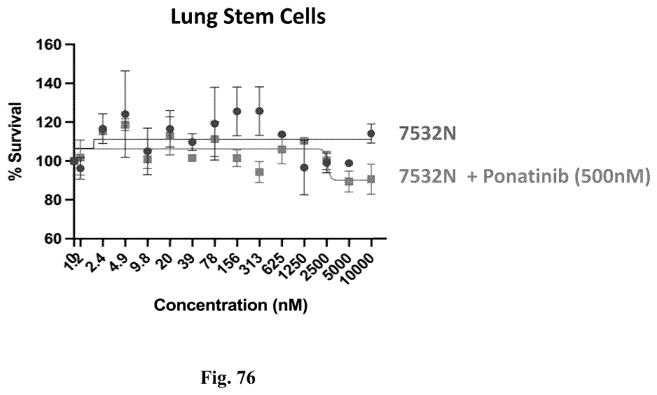

The present example demonstrates that compositions described herein are non-toxic to healthy tissues as compared to known IAP inhibitors provided in Table 1. Healthy lung cells were treated with various known compounds as well as compounds as described in the present disclosure (FIGS. 30 and 76). Additionally, healthy liver cells were treated with various known compounds as well as compounds as described in the present disclosure (FIG. 31). The present compounds did not demonstrate toxicity except at higher concentrations.

TABLE 1

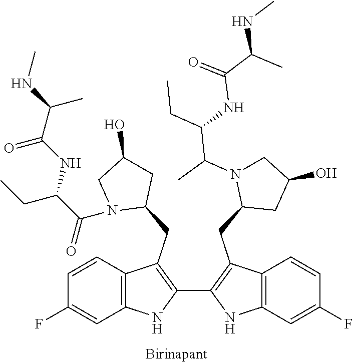 [SMILES: CC[C@H](NC(=O)[C@H](C)NC)C(=O)N1C[C@@H](O)C[C@H]1Cc1c(-c2[nH]c3cc(F)ccc3c2C[C@@H]2C[C@H](O)CN2C(C)[C@H](CC)NC(=O)[C@H](C)NC)[nH]c2cc(F)ccc12]

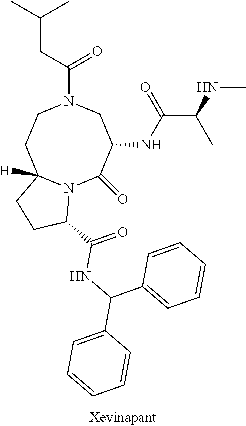 [SMILES: CN[C@@H](C)C(=O)N[C@H]1CN(C(=O)CC(C)C)CC[C@H]2CC[C@@H](C(=O)NC(c3ccccc3)c3ccccc3)N2C1=O]

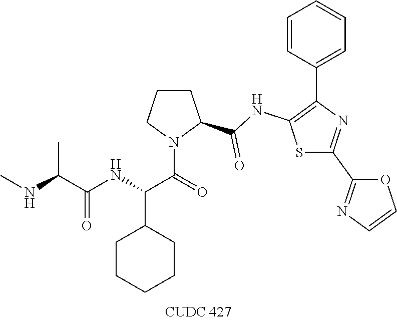 [SMILES: CN[C@@H](C)C(=O)N[C@H](C(=O)N1CCC[C@H]1C(=O)Nc1sc(-c2ncco2)nc1-c1ccccc1)C1CCCCC1]

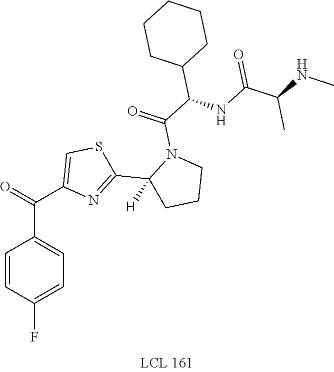 [SMILES: CN[C@@H](C)C(=O)N[C@H](C(=O)N1CCC[C@H]1c1nc(C(=O)c2ccc(F)cc2)cs1)C1CCCCC1]

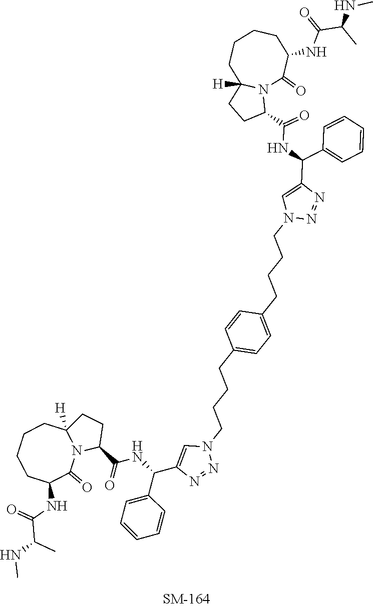 [SMILES: CN[C@@H](C)C(=O)N[C@H]1CCCC[C@H]2CC[C@@H](C(=O)N[C@@H](c3ccccc3)c3cn(CCCCc4ccc(CCCCn5cc([C@@H](NC(=O)[C@@H]6CC[C@@H]7CCCC[C@H](NC(=O)[C@H](C)NC)C(=O)N76)c6ccccc6)nn5)cc4)nn3)N2C1=O]

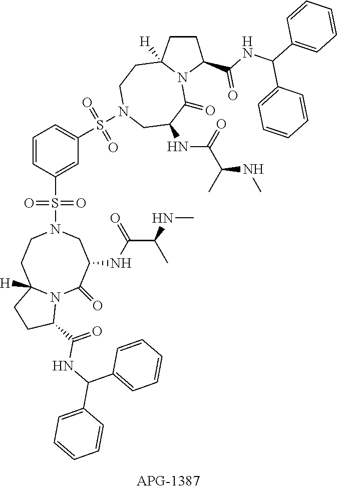 [SMILES: CN[C@@H](C)C(=O)N[C@H]1CN(S(=O)(=O)c2cccc(S(=O)(=O)N3CC[C@H]4CC[C@@H](C(=O)NC(c5ccccc5)c5ccccc5)N4C(=O)[C@@H](NC(=O)[C@H](C)NC)C3)c2)CC[C@H]2CC[C@@H](C(=O)NC(c3ccccc3)c3ccccc3)N2C1=O]

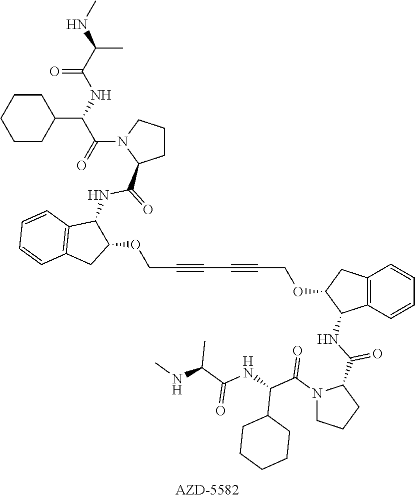 [SMILES: CN[C@@H](C)C(=O)N[C@H](C(=O)N1CCC[C@H]1C(=O)N[C@H]1c2ccccc2C[C@H]1OCC#CC#CCO[C@@H]1Cc2ccccc2[C@@H]1NC(=O)[C@@H]1CCCN1C(=O)[C@@H](NC(=O)[C@H](C)NC)C1CCCCC1)C1CCCCC1]

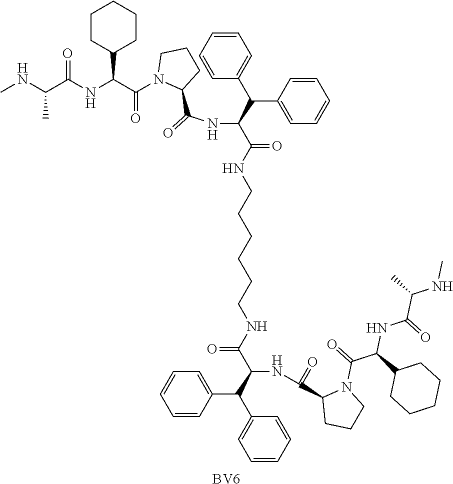

### Example 4: Methods for Testing Efficacy of Compounds Described Herein

The present example describes methods used for testing the efficacy of compounds described herein. The selective growth of stem cells of cancers, of their precursor lesions, and of normal and chronically diseased epithelial tissues is described in the references below. In brief, biopsied tissues are reduced to single cell suspensions and plated onto lawns of irradiated 3T3-J2 feeder cells in specialized StemEcho media and libraries of colonies are evident in 7-10 days. Single cell-derived clones are generated by single cell FACS sorting to 384 well plates and wells with individual colonies are expanded, analyzed by molecular genetics, and grown as discrete clones.

Approximately 400,000 cells from discrete clones relevant to particular disease states (cancer, chronic inflammatory disease) are plated into 384 plates previously seeded with irradiated feeder cells, allowed to grow for 5 days, and then exposed to the test compounds in a serial dilution format. After 2-5 days, the cells are fixed using paraformaldehyde and the human cells are labelled with human-specific antibodies followed by fluorochrome-labelled secondary antibodies. Human cell numbers are quantified via high-throughput imaging technology (CellInsight CX7 LED, Thermo), and data analyzed by Excel.

### REFERENCES

Wang X, Yamamoto Y, Wilson L H, Zhang T, Howitt B, Farrow M A, Kern F, Ning G, Yue Hong, Khor C C, Chevalier B, Bertrand D, Nagarajan N, Sylvester F A, Hyams J S, Devers T, Bronson R, Lacy D B, Ho K Y, Crum C P, McKeon F and Xian W. (2015). Cloning and variation of ground state intestinal stem cells. Nature 522, 173-178.
Yamamoto Y, Wang X, Bertrand D, Kern F, Zhang T, Hu Y Y, Deluba M, Srivastava S, Ming T, Khor C C, Wilson L, Blaszyk H, Rolshud D, Liu J J, Howitt B, Crum C P, Nagarajan N, Ho K Y, McKeon F, and Xian W. 2016. Mutational Spectrum of Barrett's Stem Cells Suggests Paths to Initiation and Progression of a Precancerous Lesion. Nat Commun. 2016 Jan. 19; 7:10380.
Qi Y, Mahalingam R, Flynn K, Rinaldi F, Liew A A, Neupane R, Vincent M, Crum C P, Ho K Y, Hou J K, Hyams J S, Sylvester F A, McKeon F, and Xian W. (2019) An Efficient Method for Cloning Gastrointestinal Stem Cells from Patients via Endoscopic Biopsies. Gastroenterol. 156 (1):20-23.
Duleba M, Yamamoto Y, Neupane R, Rao W, Xie J Z, Qi Y, Liew A A, Niroula S, Zhang Y T, Mahalingam R, Wang S, Goller K, Ajani J A, Vincent M, Ho K K, Hou J K, Hyams J S, Sylvester F A, Crum C P, McKeon F, and Xian W. (2019). Cloning of Ground State Intestinal Stem Cells from Endoscopic Biopsies. Nature Protocol. 15, 1612-1627.
W. Rao, S. Niroula, S. Wang, M. Vincent, F. McKeon, W. Xian, Protocol for Cloning Epithelial Stem Cell Variants from Human Lung. STAR Protoc 1 (2020).
Rao W, Wang S, Duleba M, Niroula S, Goller K, Xie J, et al. Regenerative metaplastic clones in COPD lung drive inflammation and fibrosis. Cell 2020, 181, 848-864.e818.
Wang S, Rao W, Hoffman A, Lin J, Li J, Lin T, et al. Cloning a profibrotic stem cell variant in idiopathic pulmonary fibrosis. Sci Transl Med. 2023 15(693):eabp9528.

### Example 5: Treatment of Precursor Cancer Lesions

The present example demonstrates the ability of compounds of the present application to treat precursor lesions of cancer.

The persistence of treatment failures for many cancers is driving efforts to preemptively target precursor lesions. The present example describes novel technology to capture rare, clonogenic cells from biopsies of esophageal adenocarcinoma (EAC) and associated precursor lesions including high-grade dysplasia (HGD), low-grade dysplasia (LGD), and Barrett's esophagus (BE). The molecular genetics of clones from patient-matched lesions reveal the mutational evolution of a cancer at unprecedented resolution, and the canonical stem cell properties of these clones extend the notion of “cancer stem cells” to rare cells of each precursor lesion. Synthetic lethal screening strategies identify drug combinations described herein both eliminate BE stem cells and favor normal esophageal stem cells. Remarkably, these drug combinations show similar efficacy toward stem cells of LGD, HGD, and EAC. Chemically optimized drug combinations show efficacy in vivo and reveal insights into the pathways by which neoplastic stem cells escape cell death.

Precursor lesions of lethal cancers represent attractive targets for the prevention of cancer¹⁻⁴. It is now clear that cancer is a late manifestation of a decades-long evolutionary process which is dominated, at least temporally, by a succession of precursor lesions⁵⁻⁹. Most colorectal cancers, for instance, initiate as small adenomas associated with APC mutations, progresses to large adenomas marked by activating KRAS mutations and a loss of epithelial polarity, and finally, with the acquisition of mutations in genes such as TP53 and SMAD4, the onset of invasive cancer⁵. The analogous process for gastric adenocarcinoma linked to chronic H. pylori infections was defined by Correa⁶ as a linear path from low- and high-risk gastric intestinal metaplasia (GIM), dysplasia, and invasive cancer, a progression driven in part by the acquisition of mutations in tumor suppressor and proto-oncogenes¹⁰⁻¹². Barrett's esophagus, the “intestinal metaplasia” precursor lesion of esophageal adenocarcinoma (EAC), progresses to cancer along a path of LGD, HGD, and finally EAC that parallels the Correa sequence for intestinal gastric adenocarcinoma⁸. While clinically-defined BE can arise without driver mutations¹³, the loss of p16 (CDKN2A) is thought to underlie the expansion of dominant clones which, in turn, evolve as multiple and competing subclones¹⁴. TP53 alteration in a subset of these clones is linked to the activation of protooncogenes, as well as the loss of additional tumor suppressors, to promote dysplasia and ultimately invasive adenocarcinoma. While much of this scenario was deduced from molecular genetic analyses of incidental samples of BE, dysplasia, or EAC from various patients, the simultaneous presence of BE and dysplasia in the distal esophagus of patients with EAC suggested the potential of reconstructing the precise mutational progression across these lesions that led to cancer^{15,16}. Bulk sequence analyses of such biopsies are beginning to show overlap single nucleotide and structural variations that unite these lesions, though clonal heterogeneity, stromal contamination, and the ongoing evolution of these precursor lesions years after spawning successive lesions remain challenges to defining the mutational progression to EAC and cancers in general¹⁷⁻¹⁹. In an effort to deconvolute the cellular and genetic heterogeneity of lesional biopsies, we applied technology that enables the cloning of normal gastrointestinal stem cells to endoscopic biopsies of BE^{13,20}. This work demonstrated that BE is dependent on a discrete population of highly immature stem cells with immense proliferative potential for its regenerative growth, and that these stem cells differentiate to an intestinal metaplasia indistinguishable from BE.

In the present example, we extend the notion that BE relies on specific stem cells to all neoplastic lesions involved in the progression to EAC. From patient-matched endoscopic biopsies of BE, LGD, HGD, and EAC, this example demonstrate that each has clonogenic cells that show unlimited proliferative potential and absolute commitment to the specific lesions from which they were derived. Importantly, these stem cell clones proved to be remarkably stable at the level of copy number variation (CNV) and single nucleotide variation (SNV) both in vitro and in vivo. This property enabled an assembly of their phylogenetic relationships to describe the evolution of EAC via successively diminishing mutational thresholds separating BE, LGD, HGD, and finally EAC. The intrinsic immortality of these stem cells has enabled high-throughput, “synthetic lethal” screening strategies directed to BE stem cells that unexpectedly identified common vulnerabilities of stem cells of neoplastic lesions across the evolution of esophageal adenocarcinoma.

### Results

Clonogenic Stem Cells from Patient-Matched Lesions

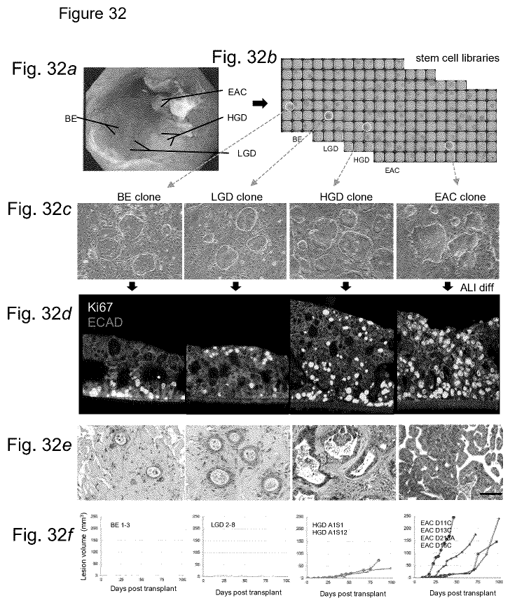

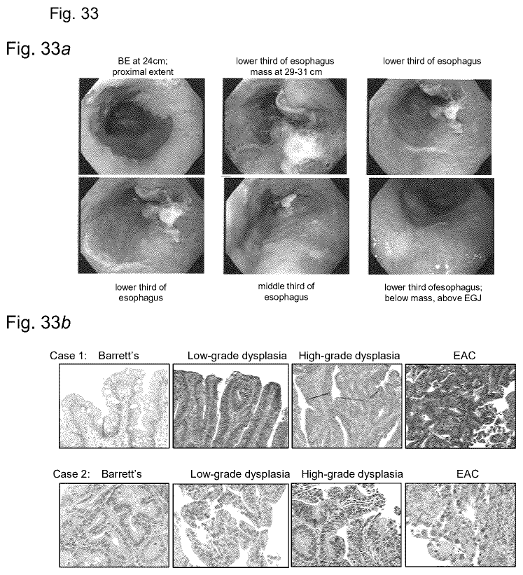

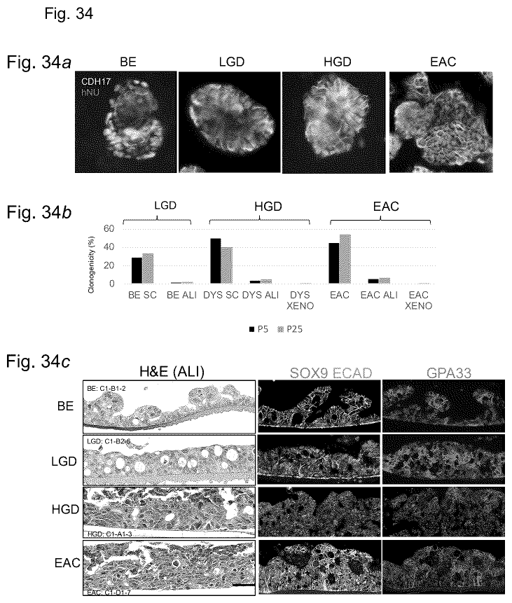

A series of 1-3 mm endoscopic biopsies from adjacent and histologically confirmed BE, LGD, HGD, and EAC lesions was obtained from therapy-naïve patients suspected of EAC (FIG. 32a; FIG. 33a,33b). Each biopsy was dissociated to yield 100,000 to 500,000 epithelial cells and plated onto lawns of irradiated 3T3-J2 fibroblasts to generate libraries of 100 to 500 epithelial colonies after 10 days of growth^{13,20,21}. The ratio of clonogenic epithelial cells from each of these lesions is approximately 1:1,000 to 1:5,000, numbers similar to the clonogenic cells from normal intestinal mucosa²⁰. Single cell-derived clones from the BE, LGD, HGD, and EAC libraries were obtained by single cell flow-sorting to 384-well plates and shown to be uniformly positive for expression of CDH17, a known marker of BE and EAC (FIG. 32b,32c; FIG. 34a). Each of these clones could be propagated as discrete lines for at least one year, during which they maintained a clonogenicity (e.g. colonies per plated cells) of between 25-50% (Extended data FIG. 34b). To further characterize these clones, their differentiation was triggered in air-liquid interface (ALI) cultures^{13,20} known to produce three-dimensional epithelia (FIG. 32d; Extended data FIG. 34c). While all clones from the BE, LGD, HGD, and EAC libraries were shared the expression of CDH17, SOX9, ECAD, and GPA33, their in vitro differentiation in ALI cultures yielded lesion-specific epithelia (FIG. 32d; FIG. 34c). BE clones gave rise to intestinal metaplasia, LGD yielded an intestinal metaplasia with reduced cellular polarity, as judged by Ki67-marked proliferation, whereas HGD and EAC clones differentiated to densely cellularized epithelia having high levels of the proliferation marker Ki67 and a general loss of cell polarity (FIG. 32d; FIG. 34c). Transplantation of these same clones into highly immunodeficient (NODscid IL2rγ^{null}; NSG) mice²² yielded nodules with histological characteristics of BE, LGD, HGD, and EAC (FIG. 1e). Despite the absence of polarity in ALI-generated epithelia from both HGD and EAC clones, the HGD clones formed limited nodules in contrast to the EAC clones that formed aggressive tumors in NSG mice (FIG. 32e,32f).

### Interclonal Heterogeneity and Clonal Genomic Stability

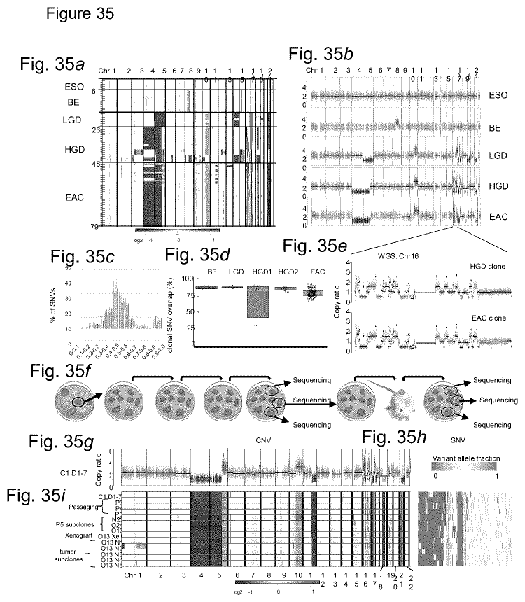

To assess clonal heterogeneity within and across these lesion-specific stem cell libraries 79, single cell-derived clones from Case 1 (6 normal esophageal, 12 BE, 8 LGD, 19 HGD, and 34 EAC clones) for expansion and low-pass, whole-genome sequencing (lpWGS; 1.6× coverage; FIG. 35a). Inspection of copy number variation (CNV) profiles showed that the normal esophageal clones lacked obvious CNV, whereas the BE, LGD, HGD, and EAC clones all showed multiple and often related CNV events. In particular, the LGD clones sustained CNV events impacting Chr. 5, 10, 17, and 21 that were also present in some of the HGD clones and all EAC clones (FIG. 35a). To examine the potential relationships between these clones in more detail, 35 of these 79 clones were selected, along with patient blood leukocytes, for whole exome sequencing (WES, 120× coverage; FIG. 35b). Single nucleotide variation (SNV) analyses of these clones showed allele frequencies that hovered around 0.5, consistent with the derivation of these clones from single cells (FIG. 35c). Importantly, most synonymous and nonsynonymous mutations harbored by a clone from a particular biopsy of BE, LGD, HGD, and EAC were shared among the independently derived clones from the same biopsy (FIG. 35d), supporting the notion that these mutations preexisted in the cells of the biopsy rather than acquired via in vitro propagation. Similarly, the sharing of CNV events within and across clones (e.g. FIG. 35a, 35b) indicate that these changes were also present in cells of the biopsies.

While these clones seemed to accurately reflect the mutational profiles of the neoplastic cells in the patient biopsies, it was less clear whether the known genomic instability of cancers^{22,23} would, over extended growth, degrade their proxy value. In this regard, we noted that some of the HGD clones and all EAC clones from Case 1 displayed a chromothripsis event²⁴ of chromosome 16 marked by complex rearrangements (e.g. FIG. 35b). Assuming that this chromothripsis event might be a sentinel for genomic instability, whole genome sequencing (WGS, 40× coverage) profiles of one HGD clone (C1-A1-3) and one EAC clone (C1-D1-7) that likely diverged several years apart in this patient were examined²⁵. Remarkably, the complex structural rearrangements of chromosome 16 assembled from WGS of these two clones were largely indistinguishable (FIG. 35e). The overall genomic stability of Case 1 clones across extensive propagation in vitro and during tumor formation following xenografts in mice was probed. The genomic profiles of individual clones (e.g. EAC clone C1-D1-7) varied over multiple cell divisions during serial passaging in vitro and after 6 weeks of tumor growth in immunodeficient mice (FIG. 35f-i). At discrete passages of in vitro cultivation, and after tumor formation as xenografts, the cells were re-cloned through the generation of libraries of clonogenic cells and by flow-sorting to single cells (FIG. 35f). DNA was collected from the derived subclones and subjected to WES (142× coverage). Importantly, neither the HGD nor EAC clones showed evidence of extensive arm-level or whole chromosome loss or gains either in vitro or during growth as xenografts in vivo (FIG. 35I). In addition, these clones showed minimal changes at the single nucleotide level within exons following long-term passaging in vitro or as xenografts in vivo, with a complete conservation of the starting 82 nonsynonymous SNPs (nsSNPs) and gain of an average of 7 nsSNPs in 50 days in culture and 3 nsSNPs during six weeks of tumor growth in mice (FIG. 35h). These data suggest that the HGD and EAC clones showed a degree of genomic stability similar to that of normal gastrointestinal stem cells²⁰ and that these clones, in aggregate, reflect the mutational profiles of the respective lesions.

Phylogenetics of Cancer from Patient-Matched Precursor Stem Cells

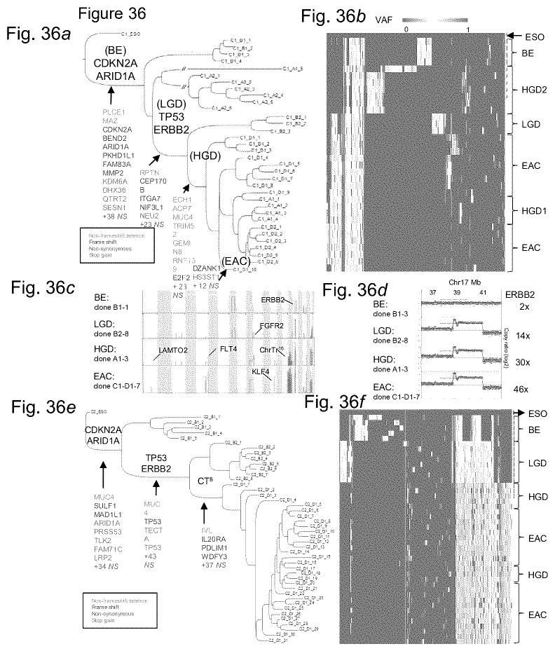

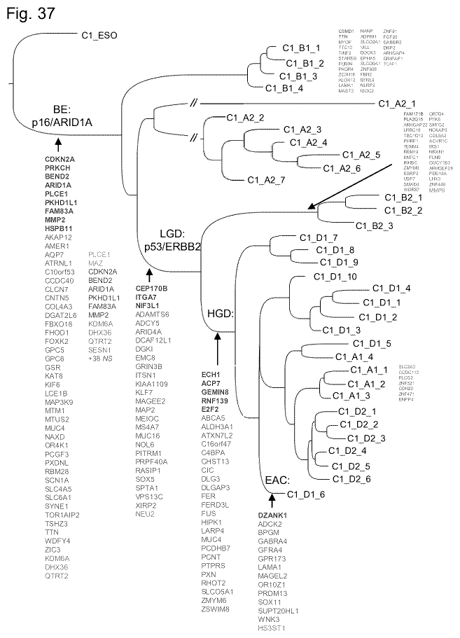

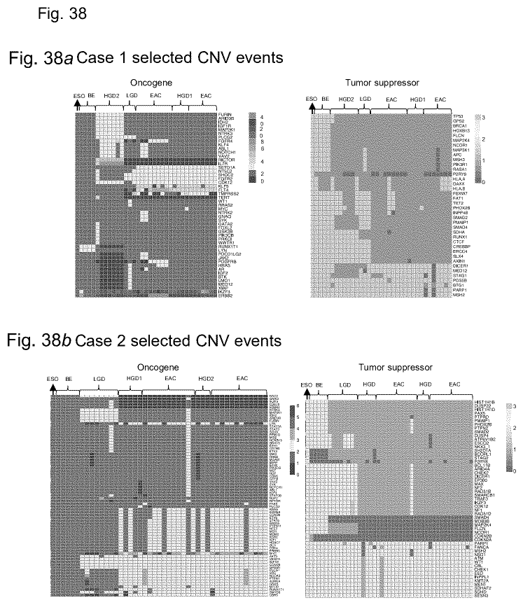

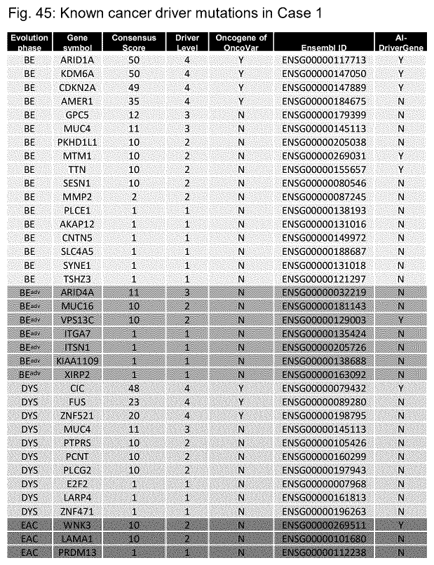

To assess the evolutionary relationships between the BE, LGD, HGD, and EAC clones, a phylogenetic analysis across the 35 clones with WES data of Case 1 based on 679 somatic SNVs (allele frequency >0.2) was performed (FIG. 36a,36b), most of which, as expected, were heterozygous mutations with variant allele fractions (VAF) around 0.5 (FIG. 36b). The resulting six clades in the phylogenetic tree suggested a common ancestor evolving into the “in-line” clades (BE, LGD, HGD1, and EAC1 and EAC2) that ultimately led to the tumor in this patient, and one additional clade (HGD2) that did not contribute to presenting tumor. In addition to CDKN2A and ARID1A mutations associated with BE^{8,9,14-16}, the BE clones harbored 48 somatically-derived, code-altering mutations (CAMs; nonsynonymous SNVs, stop-gain, and indels) that were transmitted to LGD clones, as well as many others acquired by BE clones after the generation of LGD clones (FIG. 37). We also noted an amplification of the ERBB2 locus in all LGD, HGD, and EAC clones (FIG. 36c,36d), and one with the same breakpoints in one of the four BE clones (C1-B1-1: 6×ERBB2 amplification; FIG. 36c, 36d; FIG. 38a). The LGD clones showed a more advanced mutational profile. Among changes in LGD clones that were ultimately transmitted in-line to HGD clones were TP53 mutations (via CNV), a further amplification of the ERRB2 locus to 14 copies, 28 additional CAMs, and 16 additional CNV events affecting 592 genes (FIG. 36a,36b; FIG. 37; FIG. 38a, FIG. 45). In addition to the 50 CAMs from BE and the 28 CAMs from LGD, the in-line HGD1 clones showed a chromothripsis event impacting chromosome 16 (Chr16), an additional 30 CAMs, as well as 8 new CNV events impacting 214 genes, all of which were transmitted to EAC clones. Finally, the in-line transition from HGD to EAC was accompanied by only 4 to 14 additional CAMs, a further amplification of the ERRB2 locus to 35-46 copies, and only one to three new CNV events affecting 56-389 genes (FIG. 36a-36d; FIG. 37; FIG. 38a). Importantly, all EAC clones shared 49 of the 111 CAMs found in the BE ancestor, underscoring the link between the precursor clones and the 16 EAC clones analyzed. While the clones of the HGD2 clade did not give rise to the tumor in this patient, their mutational profile involving TP53, ARID1A, CDKN2A, ERBB2, as well as a host of other genes, underscores their prospective risk for progression.

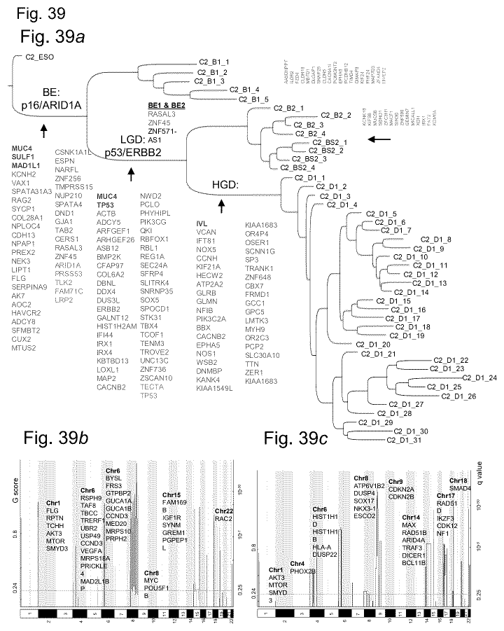

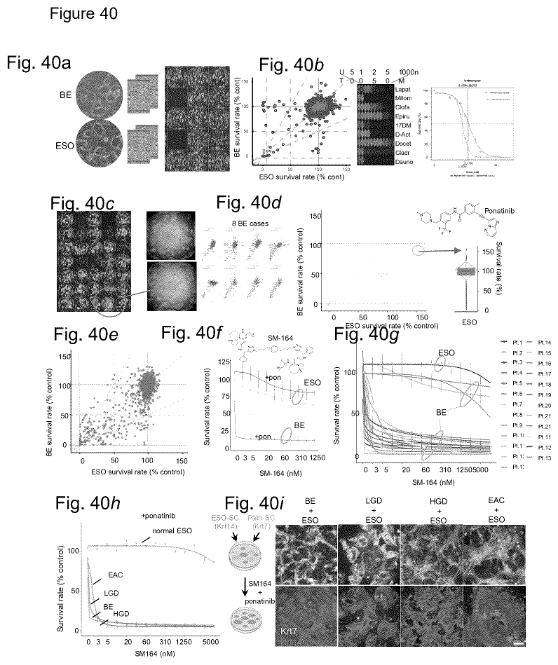

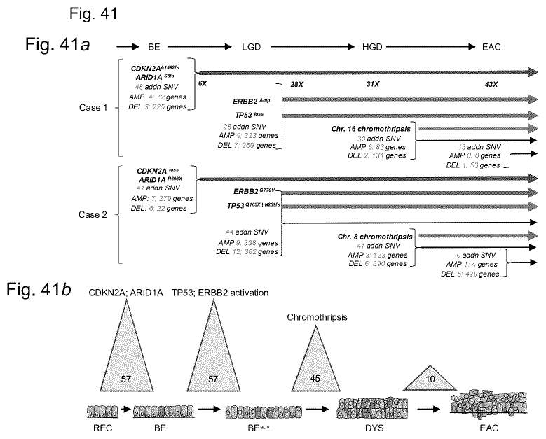

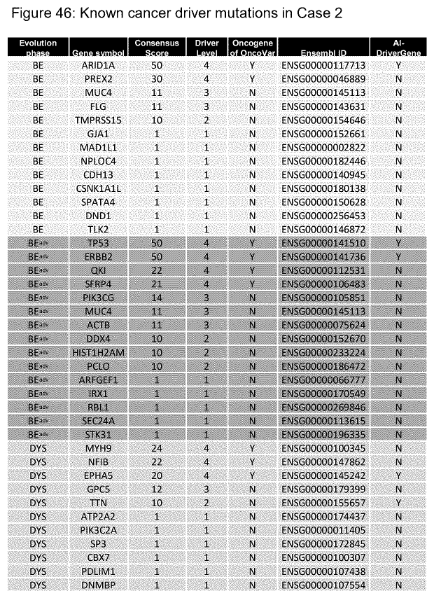

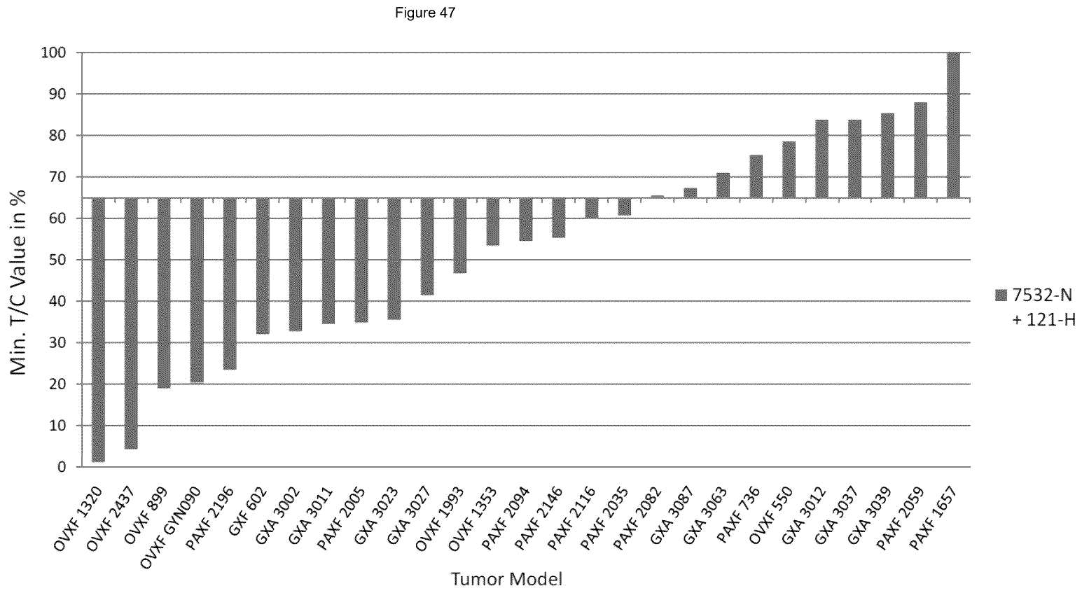

From a second case of EAC, the phylogenetic relationships between 45 clones sampled from BE, LGD, HGD, and EAC stem cell libraries based on 462 somatic SNVs were analyzed (FIG. 36e,36f, FIG. 37b; FIG. 39). These clones are linked by the absolute presence of 42 CAMs identified in BE of Case 2. As in Case 1, the BE clones of Case 2 showed a biallelic loss of CDKN2A, a non-synonymous mutation in ARID1A, which, along with 41 additional CAMs, were passed on to LGD. Like Case 1, the transition to LGD was accompanied by mutations in TP53 (stop-gain/deletion) and an activation of ERBB2, the latter via a previously characterized²⁶, nonsynonymous (G776V) mutation (FIG. 46). In addition, the LGD clones in Case 2 acquired an additional 44 CAMs, as well as 21 CNV events impacting 720 genes. The transition to HGD in Case 2, as with Case 1, was accompanied by a chromothripsis event²⁴ (Chr. 8; FIG. 40e), as well as the acquisition of 41 CAMs and 9 CNV events impacting 1013 genes together with a whole genome duplication event common to EAC²⁷. While these alterations in HGD were transmitted to EAC, the transition to EAC was remarkable for its lack of addition CAMs and the acquisition of only 6 CNV events affecting 494 genes (FIG. 36e,36f, FIG. 37b; FIG. 39). Abroad quantification of the mutational events in each lesion in Cases 1 and 2 suggest relatively high thresholds for the formation of BE and of LGD, a somewhat lower threshold to achieve HGD, and a surprisingly low number of mutational events that distinguish HGD from EAC (FIG. 41).

### Synthetic Lethal Combinations Target Stem Cells Across Neoplastic Lesions

To screen for small molecule drugs targeting stem cells of precursor lesions, we leveraged the long-term proliferative potential of these stem cells for high-throughput screens of libraries of established and experimental drugs in 384-well plates (FIG. 40a). Parallel screens of patient-matched, GFP-labelled normal esophageal stem cells and BE stem cells proved remarkably reliable (Z-factor scores ca. 8.2) and yielded multiple drug candidates including the ERBB2 inhibitor Lapatinib, the alkylating agent mitomycin C, the HSP90 inhibitor 17-DMAG, and the purine analog Cladribine. However, subsequent dose-response trials showed disappointingly small gaps between IC₅₀ values for BE versus ESO stem cells ranging from 5- to 15-fold.

Although single drug candidates showed limited selectivity, it was observed that several molecules in these screens had a growth promotion effect on normal ESO stem cells (FIG. 40c). One of these molecules, the tyrosine kinase inhibitor (TKI) ponatinib²⁸, proved to be a standout across eight patient-derived BE stem cells for its ability to promote the growth of ESO stem cells while marginally (20%) inhibiting BE stem cells (FIG. 40d). This effect attracted attention as BE and ESO can be viewed as a competitive interaction²⁹, as well as for the potential of ponatinib as the basis of a synthetic lethal strategy for combinations that could selectively target the BE stem cell. Repetition of high-throughput screens against BE and ESO stem cells in the presence of ponatinib yielded a new set of molecules that selectively targeted BE stem cells (FIG. 40e), the best of which, from dose-response analyses, were Birinapant and SM-164, members of class of SMAC/Diablo mimetics that promote the degradation of Inhibitors of Apoptosis (IAP, e.g. cIAP1, cIAP2, and xIAP)^{30,31}. Although the IAP antagonist SM-164 showed selectivity against BE stem cells versus normal ESO stem cells, this effect was greatly enhanced in the presence of ponatinib (FIG. 40f). Importantly, this combination of ponatinib and certain IAP antagonists proved to be highly effective in vitro against 20 of 23 BE stem cells derived from individual patients (FIG. 40g). Surprisingly, this same combination of ponatinib and IAP antagonist showed similar effects against stem cells from more advanced lesions of Case 1, including those of LGD, HGD, and EAC, even though these stem cells were not part of the high-throughput drug screens (FIG. 40h). The selectivity of the ponatinib/IAP antagonist combinations was further demonstrated by in vitro co-cultures of normal ESO stem cells and stem cells of BE, LGD, HGD, and EAC, which greatly favored the survival and propagation of ESO stem cells and disfavored the respective neoplastic cells (FIG. 40i).

### Advanced Synthetic Lethal Combinations

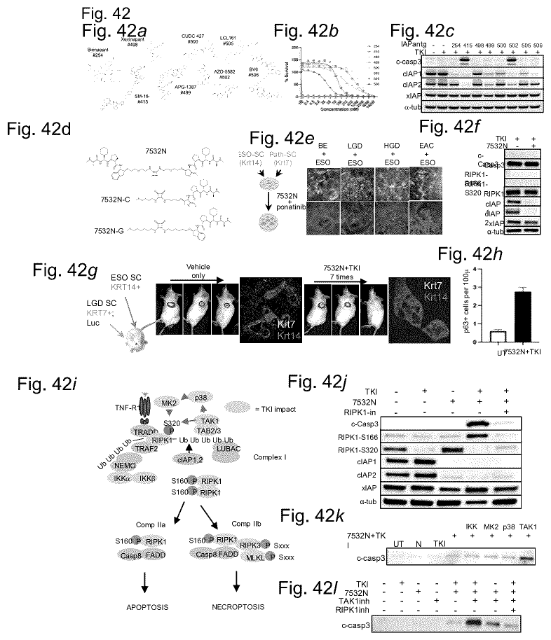

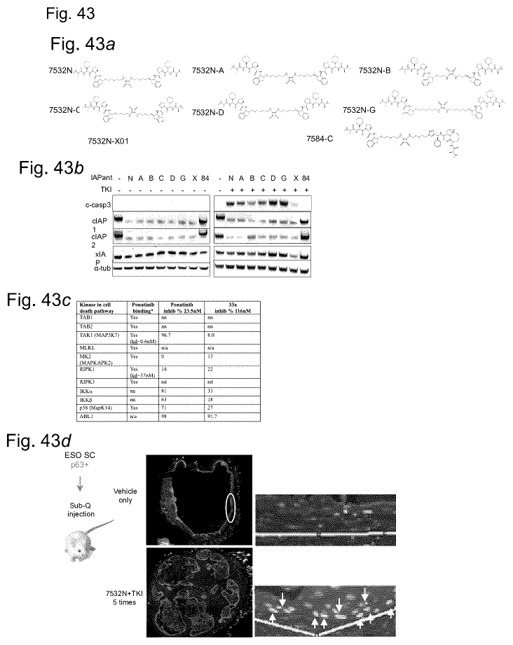

Tremendous efforts in the development of IAP antagonists have yielded a broad array of monovalent (e.g. Xevinapant, CUDC427, LCL161) and bivalent (e.g. Birinapant, SM-164, APG-1387, AZD-5582, BV6) molecules (FIG. 42a), though to date none has achieved clinical approval^{30,31}. To learn more about the relative efficacy of these IAP antagonists in a synthetic lethal combination with ponatinib, we compared their effects in dose-response assays in vitro against BE stem cells. Consistent with the improved impact of bivalent IAP antagonists relative to monovalent species³², the bivalent IAP antagonists, particularly SM-164³³ and AZD-5582³⁵, other bivalent IAP antagonists, as well as the indicated monovalent IAP antagonists, proved less effective (FIG. 5b). This pattern was reflected in western blots of treated BE stem cells exposed to the IAP antagonists and ponatinib, which showed that only SM-164 and AZD-5582 induced a cleaved caspase 3 (c-Casp3) within the six-hour duration of exposure (FIG. 42c), consistent with the degradation of cIAP1 and cIAP2. Based on the properties of diverse IAP antagonists in combination with ponatinib towards BE stem cells, synthetic chemistry was employed to expand the repertoire of IAP antagonists available for these studies. A squaramide linker strategy was chosen for generating bivalent IAP antagonists based on its planar structure and separation of the headgroups that contain the peptide memetic domains that interact with cIAP1, cIAP2, and XIAPs, and screened them in dose-response assays against BE stem cells in the presence and absence of ponatinib (FIG. 42d). A series of symmetric molecules using the headgroup of AZD-5582 and a squaramide linker of varying lengths yielded several low nanomolar IAP antagonists that induced apoptosis in BE stem cells in a ponatinib-dependent manner (FIG. 43a,43b). One of these, I-1 (7532N), along with ponatinib, proved in co-cultures of normal esophageal (ESO) stem cells and stem cells of BE, LGD, HGD, and EAC to select for the domination of normal ESO stem cells (FIG. 42e). Consistently, the treatment of normal ESO stem cells with the combination of (I-1) 7532N and ponatinib resulted in the degradation of cIAP1 and cIAP2 but not the induction of apoptosis as judged by the induction of cleaved Caspase 3 (FIG. 42f). The benign effects of the 7532N/ponatinib combination on normal ESO stem cells was underscored the effects of the combination on co-xenografts of LGD and normal ESO stem cells in mice. Seven treatments (5 mg/kg; 30 mg/kg) over three weeks not only eliminated the LGD signal but resulted in an expanded population of normal ESO stem cells and the epithelia they form (FIG. 42g,42h).

Notably, BCR-Abl inhibitors, such as imatinib, nilotinib, dasatinib, and bosutinib, were not identified in our screens. Moreover, ponatinib has been shown to be an inhibitor of both RIPK1 and RIPK3^{36,37}, “executioner” kinases proximal to distinct cell death processes including apoptosis and necroptosis. Unlike the bulk of clinically deployed BCR-ABL inhibitors, detailed interaction and inhibition studies have shown that ponatinib impacts a number of kinases and other proteins in pathways governing the onset of apoptosis and necroptosis³⁶⁻³⁸, including TAK1, TAB2, TAB3, p38MAPK, MAPKAPK2 (MK2), IKKα/IKKβ, MLKL, as well as RIPK1 and RIPK3 (FIG. 42i; Extended data FIG. 41c). Many of the kinases that ponatinib impacts have been implicated in the suppression of RIPK1 via the promotion of phosphorylation of S320 to offset the activating autophosphorylation of RIPK1 at S166³⁹. Consistently, treatment of LGD stem cells with ponatinib alone results in the loss of S320 phosphorylation without the induction of apoptosis (FIG. 42j), and the treatment of these cells with the novel IAP antagonist 7532N leads to a degradation of cIAP1 and cIAP2 without inducing cleaved Caspase 3 or either the inhibitory (Ser320) or activating (Ser166) phosphorylation of RIPK1. However, treatment with the combination of 7532N and ponatinib (FIG. 42f) showed a loss of the inhibitory phosphorylation of RIPK1 at S320, the gain of activating phosphorylation of RIPK1 at S166, and the induction of apoptosis in a process blocked by a RIPK1 inhibitor but not a RIPK3 inhibitor.

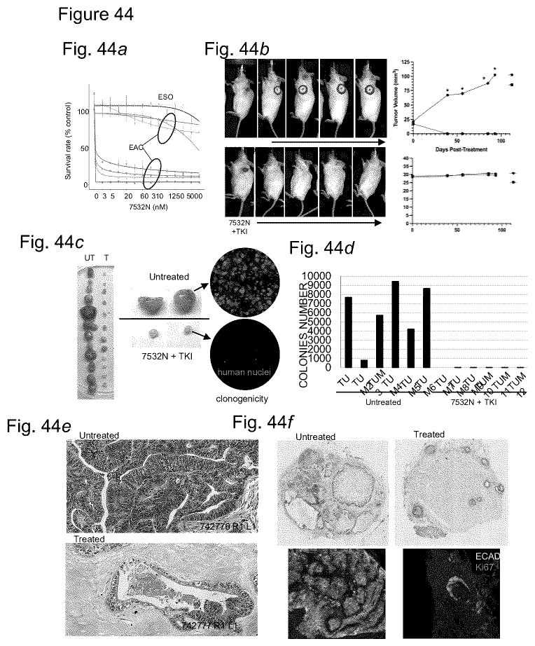

The IAP antagonistic/ponatinib combination in vitro proved effective against 5 of 8 EAC stem cells from eight cases of EAC, with the outliers showing profound resistance (FIG. 44a). To test the 7532N/ponatinib combination against EAC stem cell in vivo, the EAC stem cell clones were labeled with luciferase via retroviral transduction and performed subcutaneous injections in NSG mice to generate tumors. Intraperitoneal treatment of these mice with 7532N/ponatinib (15 mg/Kg; 30 mg/Kg) three times per week for three weeks rendered these tumors undetectable by luciferase or palpitation after 20 days (9 treatments), and the mice tolerated an additional 19 treatments without obvious weight loss (FIG. 44b,44c). Clonogenicity assays of the xenografted tumors showed a 400-fold differential in EAC stem cell colonies in the untreated tumors versus those treated with 7532N/ponatinib (FIG. 44d,44e). By histology, the remaining tumor cells in the mice treated for three weeks showed a washed out appearance compared to the untreated mice (FIG. 44f). Consistently, analysis of the cell proliferation marker Ki67, which showed robust activity the untreated tumors, was greatly reduced in cells remaining in the treated tumors.

### Discussion

The present example applied single cell cloning technology to patient-matched lesions implicated in the emergence of EAC. The salient features of these cloned cells, including their high clonogenicity, unlimited proliferative capacity, and absolute fate commitment to the respective BE, LGD, HGD, and EAC lesions both in vitro and in vivo, functionally define them as stem cells for these oncogenic lesions. As such, these clonogenic cells provide additional support for the cancer stem cell hypothesis⁴⁰⁻⁴² and generalizes this concept to the successive precursor lesions leading to EAC.

A striking feature of the HGD and EAC clones was their genomic stability reflected by limited CNV and SNV events during either long-term propagation in vitro or growth as xenografts in immunodeficient mice, a property that enhances the in vitro value of these clones as proxies of the cells in the respective lesions. The genomic stability of these clones suggests that the well-documented genomic instability of tumors is a reflection of the dynamics of a heterogeneous population, whereas individual cells within the population, absent specific mutagenic syndromes⁴³, might be more staid. The stability of these clones, coupled with the obvious clonal heterogeneity displayed by all lesions in EAC tumorigenesis, underscores the likelihood of one or more “big-bang” events⁴⁴ generates the observed intralesional diversity.

The parallels seen in the respective transitions of EAC clones studied from BE, LGD, HGD, suggest patterns that likely have clinical correlates. For instance, BE is a common clinical finding in an estimated 1-3% of individuals in Western countries and yet the risk of progression of BE to EAC is low (ca. 0.2% per year)^{25,45-47}. In contrast, HGD is considered to have a very high risk of progression to EAC (ca. 10% per yr) and triggers immediate intervention in the form of radiofrequency ablation (RFA) or endoscopic mucosal resection (EMR)^{25,45-50}. The low risk assessment of BE is consistent with the behavior and mutational profile of the BE clones (non-tumorigenic; wild type TP53, absence of amplified protooncogenes), as is the high risk conferred by HGD clones marked by mutant TP53, multiple oncogene amplifications, chromothripsis and genome duplication. The minimal genomic differences between HGD and EAC underscores the threat posed by dysplastic lesions for near-term conversion to metastatic cancer

Aside from low-risk BE and high-risk HGD, there is much clinical interest in the presence of the LGD intermediate as a harbinger for progression⁴⁵⁻⁴⁷. While the inter-observer agreement for LGD can be low, there is general agreement that LGD has an enhanced risk (0.4-13.4%/year) for progression to HGD and ultimately EAC. The LGD clones identified from both EAC cases examined here display a partial loss of polarity upon differentiation in 3-D cultures, and show a mutational profile (loss of TP53 function, oncogene activation, multiple CAMs and CNV events) consistent with enhanced risk over BE. These findings underscore the need for LGD detection methods as well as the development of agents that target LGD stem cells in the prevention of EAC.

This work supports the notion that rare (ca. 1:1,000), lesion-specific stem cells, as with stem cells of any regenerative epithelia, play a disproportionate role in the future growth of BE, LGD, HGD, and EAC and thus represent important therapeutic targets. Efforts to target these stem cells, enabled by their high proliferative potential, failed to identify promising single agents. In contrast, synthetic lethal strategies yielded highly effective drug combinations against BE stem cells that, unexpectedly, targeted a common vulnerability of stem cells from each lesion in the progression to EAC. The best synthetic lethal drug combination, consisting of an IAP antagonist and a tyrosine kinase inhibitor (TKI), induced a form of apoptotic cell death that was dependent on the executioner kinase RIPK1, but not on RIPK3. Importantly, this synthetic lethal combination targets distinct aspects of Complex 1, a multiprotein assembly integrates diverse signaling inputs and simultaneously governs alternative cell death, cell survival, and inflammatory outputs⁵¹. IAP antagonists, for instance, destabilizes Complex 1 by promoting the loss of cIAP1 and cIAP2, two ubiquitin ligases that contribute to a RIPK1 and NF-kB regulatory scaffold involving TAK1, TAB1, TAB2, and TAB3, as well as IKKα, IKKβ, and NEMO^{30,31,51}. A less obvious component of the synthetic lethal combination was ponatinib, an ABL kinase inhibitor approved for the treatment of BCR-ABL+blood cancers⁵². Ironically, unlike other approved ABL kinase inhibitors including imatinib, nilotinib, dasatinib, and bosutinib, ponatinib has been shown to inhibit both RIPK1 and RIPK3, and therefore represents a dual inhibitor of apoptosis and necroptosis^{36,37}. However, also unlike other ABL kinase inhibitors, ponatinib impacts a broad array of kinases linked to Complex 1, including TAK1, TAB1, TAB2, p38MAPK, and MK2, many of which have been implicated in the negative regulation of RIPK1³⁶⁻³⁸. The synthetic lethal and pro-apoptotic effects of combination towards stem cells of the BE, LGD, HGD, and EAC lesions clearly is mechanistically complex but involves the ability of ponatinib to simultaneously repress the network of kinases that maintain RIPK1 in a latent state. This derepression of RIPK1 activity must overcome the coincidental inhibition of RIPK1 by ponatinib itself to drive a RIPK1-dependent apoptosis while preventing, at the same time via RIPK3 inhibition, the onset of necroptosis. Lastly, we should note that the same synthetic lethal drug combination that targets BE, LGD, HGD, and EAC imparts a benign and indeed a positive proliferative effect on normal esophageal stem cells that typically surround the precursor lesions. This bodes well for the preemptive treatment of early lesions such as BE and LGD, whereby adjacent normal ESO stem cells could contribute to the repair of the mucosal epithelia. We anticipate that such drug combinations and their continued development lead to clinically effective treatments to prevent or treat EAC.

### REFERENCES

1. Shain, A. H. et al. The Genetic Evolution of Melanoma from Precursor Lesions. N Engl J Med 373, 1926-1936, doi:10.1056/NEJMoa1502583 (2015).
2. Fischer, C. G. & Wood, L. D. From somatic mutation to early detection: insights from molecular characterization of pancreatic cancer precursor lesions. J Pathol 246, 395-404, doi:10.1002/path.5154 (2018).
3. Vogelstein, B. et al. Genetic alterations during colorectal-tumor development. N Engl J Med 319, 525-532, doi:10.1056/NEJM198809013190901 (1988).
4. Yokoyama, A. et al. Age-related remodelling of oesophageal epithelia by mutated cancer drivers. Nature 565, 312-317, doi:10.1038/s41586-018-0811-x (2019).
5. Fearon, E. R. & Vogelstein, B. A genetic model for colorectal tumorigenesis. Cell 61, 759-767, doi:10.1016/0092-8674(90)90186-i (1990).
6. Correa, P. Human gastric carcinogenesis: a multistep and multifactorial process—First American Cancer Society Award Lecture on Cancer Epidemiology and Prevention. Cancer Res 52, 6735-6740 (1992).
7. Vogelstein, B. & Kinzler, K. W. The Path to Cancer—Three Strikes and You're Out. N Engl J Med 373, 1895-1898, doi:10.1056/NEJMp1508811 (2015).
8. Killcoyne S, Fitzgerald R C. Evolution and progression of Barrett's oesophagus to oesophageal cancer. Nat Rev Cancer. 2021 November; 21(11):731-741.
9. Nowicki-Osuch K, Zhuang L, Jammula S, Bleaney C W, Mahbubani K T, Devonshire G, Katz-Summercorn A, Eling N, Wilbrey-Clark A, Madissoon E, Gamble J, Di Pietro M, O'Donovan M, Meyer K B, Saeb-Parsy K, Sharrocks A D, Teichmann S A, Marioni J C, Fitzgerald R C Molecular phenotyping reveals the identity of Barrett's esophagus and its malignant transition. Science. 2021 Aug. 13; 373(6556):760-767.
10. Cancer Genome Atlas Research, N. Comprehensive molecular characterization of gastric adenocarcinoma. Nature 513, 202-209, doi:10.1038/nature13480 (2014).
11. Huang K K, Ma H, Chong R H H, Uchihara T, Lian B S X, Zhu F, Sheng T, Srivastava S, Tay S T, Sundar R, Tan A L K, Ong X, Lee M, Ho S W T, Lesluyes T, Ashktorab H, Smoot D, Van Loo P, Chua J S, Ramnarayanan K, Lau L H S, Gotoda T, Kim H S, Ang T L, Khor C, Lee J W J, Tsao S K K, Yang W L, Teh M, Chung H, So J B Y, Yeoh K G, Tan P.; Singapore Gastric Cancer Consortium. Spatiotemporal genomic profiling of intestinal metaplasia reveals clonal dynamics of gastric cancer progression. Cancer Cell. 2023 Dec. 11; 41(12):2019-2037.e8.
12. Yeoh K G, Tan P. Mapping the genomic diaspora of gastric cancer. Nat Rev Cancer. 2022 February; 22(2):71-84.
13. Yamamoto, Y et al. Mutational spectrum of Barrett's stem cells suggests paths to initiation of a precancerous lesion. Nat Commun 7, 10380, doi:10.1038/ncomms10380 (2016).
14. Reid, B. J., Paulson, T. G. & Li, X. (2015). Genetic Insights in Barrett's Esophagus and Esophageal Adenocarcinoma. Gastroenterology 149, 1142-1152 e1143, doi:10.1053/j.gastro.2015.07.010.
15. Stachler, M. D. et al. Paired exome analysis of Barrett's esophagus and adenocarcinoma. Nat Genet 47, 1047-1055, doi:10.1038/ng.3343 (2015).
16. Ross-Innes, C. S. et al. Whole-genome sequencing provides new insights into the clonal architecture of Barrett's esophagus and esophageal adenocarcinoma. Nat Genet 47, 1038-1046, doi:10.1038/ng.3357 (2015).
17. Gerlinger, M. et al. Intratumor heterogeneity and branched evolution revealed by multiregion sequencing. N Engl J Med 366, 883-892, doi:10.1056/NEJMoa1113205 (2012).
18. Martincorena, I. et al. Tumor evolution. High burden and pervasive positive selection of somatic mutations in normal human skin. Science 348, 880-886, doi:10.1126/science.aaa6806 (2015).
19. McGranahan, N. & Swanton, C. Clonal Heterogeneity and Tumor Evolution: Past, Present, and the Future. Cell 168, 613-628, doi:10.1016/j.cell.2017.01.018 (2017).
20. Wang, X. et al. Cloning and variation of ground state intestinal stem cells. Nature 522, 173-178, doi:10.1038/nature14484 (2015).
21. Duleba, M. et al. An Efficient Method for Cloning Gastrointestinal Stem Cells From Patients via Endoscopic Biopsies. Gastroenterology 156, 20-23, doi:10.1053/j.gastro.2018.08.062 (2019).
22. Ben-David, U., Beroukhim, R. & Golub, T. R. Genomic evolution of cancer models: perils and opportunities. Nat Rev Cancer 19, 97-109, doi:10.1038/s41568-018-0095-3 (2019).
23. Sansregret L, Vanhaesebroeck B, Swanton C. Determinants and clinical implications of chromosomal instability in cancer. Nat Rev Clin Oncol. 2018 March; 15(3):139-150.
24. Stephens, P. J. et al. Massive genomic rearrangement acquired in a single catastrophic event during cancer development. Cell 144, 27-40, doi:10.1016/j.cell.2010.11.055 (2011).
25. Prasad G A, Bansal A, Sharma P, Wang K K. Predictors of progression in Barrett's esophagus: current knowledge and future directions. Am J Gastroenterol. 2010 July; 105(7):1490-1502.
26. Nagano, M. et al. High-Throughput Functional Evaluation of Variants of Unknown Significance in ERBB2. Clin Cancer Res 24, 5112-5122, doi:10.1158/1078-0432.CCR-18-0991 (2018).
27. Carter, S. L. et al. Absolute quantification of somatic DNA alterations in human cancer. Nat Biotechnol 30, 413-421, doi:10.1038/nbt.2203 (2012).
28. Tan, F. H., Putoczki, T. L., Stylli, S. S. & Luwor, R. B. Ponatinib: a novel multi-tyrosine kinase inhibitor against human malignancies. Onco Targets Ther 12, 635-645, doi:10.2147/OTT.S189391 (2019).
29. Wang et al., 2011 barrett's
30. Gyrd-Hansen M, Meier P. IAPs: from caspase inhibitors to modulators of NF-kappaB, inflammation and cancer. Nat Rev Cancer. 2010 August; 10(8):561-74.
31. Fulda S. Promises and Challenges of Smac Mimetics as Cancer Therapeutics. Clin Cancer Res. 2015 Nov. 15; 21(22):5030-6.
32. Cong H, Xu L, Wu Y, Qu Z, Bian T, Zhang W, Xing C, Zhuang C. Inhibitor of Apoptosis Protein (IAP) Antagonists in Anticancer Agent Discovery: Current Status and Perspectives. J Med Chem. 2019 Jun. 27; 62(12):5750-5772.
33. Lu J, Bai L, Sun H, Nikolovska-Coleska Z, McEachern D, Qiu S, Miller R S, Yi H, Shangary S, Sun Y, Meagher J L, Stuckey J A, Wang S. SM-164: a novel, bivalent Smac mimetic that induces apoptosis and tumor regression by concurrent removal of the blockade of cIAP-1/2 and XIAP. Cancer Res. 2008 Nov. 15; 68(22):9384-93.
34. Moon E S, Elvas F, Vliegen G, De Lombaerde S, Vangestel C, De Bruycker S, Bracke A, Eppard E, Greifenstein L, Klasen B, Kramer V, Staelens S, De Meester I, Van der Veken P, Rosch F. Targeting fibroblast activation protein (FAP): next generation PET radiotracers using squaramide coupled bifunctional DOTA and DATA(5m) chelators. EJNMMI Radiopharm Chem. 2020 Jul. 29; 5(1):19.
35. Hennessy E J, Adam A, Aquila B M, Castriotta L M, Cook D, Hattersley M, Hird A W, Huntington C, Kamhi V M, Laing N M, Li D, MacIntyre T, Omer C A, Oza V, Patterson T, Repik G, Rooney M T, Saeh J C, Sha L, Vasbinder M M, Wang H, Whitston D. Discovery of a novel class of dimeric Smac mimetics as potent IAP antagonists resulting in a clinical candidate for the treatment of cancer (AZD5582). J Med Chem. 2013 Dec. 27; 56(24):9897-919.
36. Fauster A, Rebsamen M, Huber K V, Bigenzahn J W, Stukalov A, Lardeau C H, Scorzoni S, Bruckner M, Gridling M, Parapatics K, Colinge J, Bennett K L, Kubicek S, Krautwald S, Linkermann A, Superti-Furga G. A cellular screen identifies ponatinib and pazopanib as inhibitors of necroptosis. Cell Death Dis. 2015 May 21; 6(5):e1767.
37. Najjar M, Suebsuwong C, Ray S S, Thapa R J, Maki J L, Nogusa S, Shah S, Saleh D, Gough P J, Bertin J, Yuan J, Balachandran S, Cuny G D, Degterev A. Structure guided design of potent and selective ponatinib-based hybrid inhibitors for RIPK1. Cell Rep. 2015 Mar. 24; 10(11):1850-60.
38. Hnatiuk A P, Bruyneel A A N, Tailor D, Pandrala M, Dheeraj A, Li W, Serrano R, Feyen D A M, Vu M M, Amatya P, Gupta S, Nakauchi Y, Morgado I, Wiebking V, Liao R, Porteus M H, Majeti R, Malhotra S V, Mercola M. Reengineering Ponatinib to Minimize Cardiovascular Toxicity. Cancer Res. 2022 Aug. 3; 82(15):2777-2791.
39. Liu Z, Chan F K. Regulatory mechanisms of RIPK1 in cell death and inflammation. Semin Cell Dev Biol. 2021 January; 109:70-75.
40. Reya, T., Morrison, S. J., Clarke, M. F. & Weissman, I. L. Stem cells, cancer, and cancer stem cells. Nature 414, 105-111, doi:10.1038/35102167 (2001).
41. Kreso, A. & Dick, J. E. Evolution of the cancer stem cell model. Cell Stem Cell 14, 275-291, doi:10.1016/j.stem.2014.02.006 (2014).
42. Batlle E, Clevers H. Cancer stem cells revisited. Nat Med. 2017 Oct. 6; 23(10):1124-1134.
43. Thibodeau S N, Bren G, Schaid D. Microsatellite instability in cancer of the proximal colon. Science. 1993 May 7; 260(5109):816-9.
44. Sottoriva A, Kang H, Ma Z, Graham T A, Salomon M P, Zhao J, Marjoram P, Siegmund K, Press M F, Shibata D, Curtis C. A Big Bang model of human colorectal tumor growth. Nat Genet. 2015 March; 47(3):209-16.
45. Song, K. Y et al. Persistent confirmed low-grade dysplasia in Barrett's esophagus is a risk factor for progression to high-grade dysplasia and adenocarcinoma in a US Veterans cohort. Dis Esophagus 33, doi:10.1093/dote/doz061 (2020).
46. Krishnamoorthi, R. et al. Risk of progression in Barrett's esophagus indefinite for dysplasia: a systematic review and meta-analysis. Gastrointest Endosc 91, 3-10 e13, doi:10.1016/j.gie.2019.07.037 (2020).
47. Kestens, C., Offerhaus, G. J., van Baal, J. W. & Siersema, P. D. Patients With Barrett's Esophagus and Persistent Low-grade Dysplasia Have an Increased Risk for High-grade Dysplasia and Cancer. Clin Gastroenterol Hepatol 14, 956-962 e951 (2016).
48. Rastogi, A. et al. Incidence of esophageal adenocarcinoma in patients with Barrett's esophagus and high-grade dysplasia: a meta-analysis. Gastrointest Endosc 67, 394-398, (2008).
49. Shaheen, N. J. et al. Radiofrequency ablation in Barrett's esophagus with dysplasia. N Engl J Med 360, 2277-2288 (2009).
50. Tomizawa, Y et al. Safety of endoscopic mucosal resection for Barrett's esophagus. Am J Gastroenterol 108, 1440-1447 (2013).
51. Clucas J, Meier P. Roles of RIPK1 as a stress sentinel coordinating cell survival and immunogenic cell death. Nat Rev Mol Cell Biol. 2023 November; 24(11):835-852.
52. Gao Y, Ding Y, Tai X R, Zhang C, Wang D. Ponatinib: An update on its drug targets, therapeutic potential and safety. Biochim Biophys Acta Rev Cancer. 2023 September; 1878(5):188949.

### Example 6: Materials and Methods

In Vitro Stem Cell Cloning from Patient-Matched Endoscopic Biopsies

Under informed consent and IRB-approved protocols at the MD Anderson Cancer Center (IRB 5 IRB00006023; LAB01-543) and the University of Connecticut Health Sciences Center (16-065-03), we obtained therapy-naive samples of esophageal adenocarcinoma (EAC) and its precursor lesions. Cases 1 and 2 were from 1 mm endoscopic biopsies of adjacent lesions deemed to be EAC, Dysplasia, and Barrett's together with normal esophageal mucosa. Tissue from Case 3 was from a metastatic pleural effusion from a primary EAC obtained. Biopsies or pleural effusion cells were dissociated to single cells as described (Yamamoto et al., 2016; Wang et al., 2015; Duleba et al., 2019) by digestion in 1 mg/ml collagenase type IV (Gibco, USA) at 37° C. for 30-45 min with agitation. Dissociated cells were passed through a 70 μm Nylon mesh (Falcon, USA) to remove aggregates, washed five times in cold F12 media, and seeded onto a feeder layer of lethally irradiated 3T3-J2 cells in StemECHO media (Multiclonal Therapeutics, Hartford, CT, USA) and grown at 37° C. in a 7.5% CO₂ incubator with media change every 2 days. Colonies appearing in 10 days were digested by TrypLE Express solution (Gibco, USA) for 10-15 min at 37° C. and cell suspensions were passed through 30 μm filters (Miltenyi Biotec, Germany) before passaging onto new feeder lawns. Single cell cloning was performed by fine tip pipetting or by flow sorting into 384-well plates previously seeded with irradiated 3T3-J2 cells.

### Stem Cell Differentiation

Air-liquid interface (ALI) cultures was used to assess stem cell differentiation potential (Yamamoto et al., 2016; Wang et al., 2015). Transwell inserts (Corning Incorporated, USA) were coated with 20% Matrigel (BD biosciences, USA) and incubated at 37° C. for 10 min to polymerize. 200,000 irradiated 3T3-J2 cells were seeded to each Transwell insert (Corning) and incubated at 37° C., 7.5% CO₂ incubator overnight. QuadroMACS Starting Kit (LS) (Miltenyi Biotec, Germany) was used to purify the stem cells by removal of feeder cells. 300,000 stem cells were seeded into each Transwell insert and cultured with stem cell media. At confluency (5 days), the apical media on the inserts was removed through careful pipetting and the cultures were continued in differentiation media (stem cell media without nicotinamide) for an additional 8-14 days prior to harvesting. The differentiation media was changed every one or two days.

### Xenografts in Immunodeficient Mice

All animal experiments were performed in accordance with Institutional Animal Care and Use Committee (IACUC)-approved protocol 16-002 at the University of Houston. Three million stem cells were kept on ice and mixed well with 50% Matrigel (Becton Dickinson, Palo Alto, USA) to a volume of 150 ul and injected subcutaneously in NSG (NODscid IL2ra^{null} Pearson et al., 2008) mice (Jackson Laboratories, Bar Harbor, USA). Xenograft size was measured with calipers and the volume was determined by the following formula: tumor volume (mm³)=½×A (mm)×B² (mm²), where ‘A’ represent the largest dimension and ‘B’ indicates the smallest dimension.

### Histology and Staining

Histology, hematoxylin and eosin (H&E) staining, rhodamine staining, Alcian blue staining (VECTOR, USA) and immunofluorescence staining were performed using standard techniques. For immunofluorescence, 4% paraformaldehyde-fixed, paraffin embedded tissue slides were subjected to antigen retrieval in citrate buffer (pH 6.0, Sigma-Aldrich, USA) at 120° C. for 20 min, and a blocking procedure was performed with 5% bovine serum albumin (BSA, Sigma-Aldrich, USA) and 0.05% Triton X-100 (Sigma-Aldrich, USA) in DPBS (−) (Gibco, USA) at room temperature for 1 hour and then reacted with primary antibodies at 4° C. overnight. The sources of primary antibodies used in this study include: rabbit monoclonal Ki67 (1:500, ab16667, Abcam), rabbit polyclonal Laminin (1:500, ab11575, Abcam), mouse monoclonal Cdh17 (1:300, SC74209, Santa Cruz Biotechnology), goat polyclonal E-Cadherin (1:500, AF648, R&D Systems). All images were captured by using the Inverted Eclipse Ti-Series (Nikon, Japan) microscope with Lumencor SOLA light engine and Andor Technology Clara Interline CCD camera and NIS-Elements Advanced Research v.4.13 software (Nikon, Japan) or LSM 780 confocal microscope (Carl Zeiss, Germany) with LSM software. Bright field cell culture images were obtained on an Eclipse TS100 microscope (Nikon, Japan) with Digital Sight DSFilcamera (Nikon, Japan) and NIS-Elements F3.0 software (Nikon, Japan).

### DNA Content Analysis

Stem cells were harvested and washed twice with cold phosphate-buffered saline (PBS). After fixation in 70% cold ethanol at −20° C. for at least 1.5 h, the samples were stained using Propidium Iodide Flow Cytometry Kit (ab139418) and then analyzed on a SH800 FACS Cell Sorter (Sony, Japan).

### Whole Exome Sequencing (WES)

For exome capture and high-throughput sequencing, about lug of genomic DNA was isolated using QIAGEN kits. The genomic DNA was sheared, end-repaired, A-tailed, adaptor-ligated, and Exome captured using the Agilent SureSelect Human All Exon V6 Kit (Agilent Technologies, CA, USA) following the manufacturer's instructions. In short, fragmentation was conducted by hydrodynamic shearing system (Covaris, Massachusetts, USA) to generate 180-280 bp fragments. Remaining overhangs were converted into blunt ends via exonuclease/polymerase activities. After adenylation of 3′ ends of DNA fragments, adapters were ligated. Fragments with ligated adapters on both ends were selectively enriched in a PCR reaction. Captured libraries were enriched in a PCR reaction to add indexes to prepare for hybridization. Products were purified using AMPure XP system (Beckman Coulter, Beverly, USA) and quantified with the Agilent high-sensitivity DNA assay on the Agilent Bioanalyzer 2100 System. The multiplexed libraries were sequenced on Illumina HiSeq X platform (150 bp paired-end reads, Illumina, California, USA). The clusters that do not pass the Chastity filter were removed from downstream analysis. At least 20 million paired reads were generated from each sample.

### Low-Pass Whole Genome Sequencing (WGS)

Sequencing libraries were prepared by TruSeq Nano DNA HT Sample Prep Kit (Illumina, California, USA) following the manufacturer's protocol. First, 1000 ng of genomic DNA was fragmented by sonication to 350 bp. Then fragments were end-repaired, A-tailed and adaptor-ligated, followed by further PCR reactions. After purification using the AMPure XP system (Beckman Coulter, Beverly, USA), the library was size-selected using Agilent 2100 Bioanalyzer and quantified by real-time PCR. The clustering of the index-coded samples was performed on a cBot Cluster Generation System using Hiseq PE Cluster Kit (Illumina, California, USA) according to the manufacturer's protocol. Next, the libraries were sequenced on Illumina Hiseq X platform (Illumina, California, USA) in 150 bp paired-end model. At least 20 million paired-end reads were generated from each sample.

### Whole Genome Exome Sequencing Data Analysis

The raw sequencing data were quality controlled to trim low-quality bases by using Trim Galore (http://www.bioinformatics.babraham.ac.uk/projects/trim_galore/). Murine sequences were filtered by Xenome (Conway et al., 2012) version 1.0.1 with default parameters. The remaining reads were aligned to reference genome (UCSC hg19) via BWA-mem (Li and Durbin, 2010) version 0.7.15 under the mem model requiring map quality >=40 (Phred-value). PCR duplicates were removed using Picard Toolkit (http://broadinstitute.github.io/picard/). GATK version 3.8.0 was used to realign reads near indels (Mills_and_1000G_gold_standard indels), and to recalibrate base quality values with default settings following the best practices pipeline of GATK (DePristo et al. 2011). Somatic SNVs and Indels were called by using LoFreq (Wilm et al., 2012) with default parameter and annotated by ANNOVAR (Yang and Wang, 2015). Based on the exonic somatic SNVs with allele frequency over 0.2, the phylogenetic trees were constructed by using SiFit (Zafar, et al. 2017) with the iterations number of 10000, which employs a heuristic search algorithm to infer the Maximum Likelihood (ML) phylogenetic tree under a finite-site model of evolution. The somatic copy number variants were called by using CNVkit genome-wide copy number variants calling pipeline (Talevich et al., 2016). The genes targeted by the significant somatic copy-number alterations that drive cancer growth were identified by GISTIC2.0 (Mermel et al. 2011). The oncogenes and tumor suppressors were fetched from the OncoKB database (Chakravarty et al., 2017). The cancer drive mutations were obtained from the OncoVar database (Wang et al., 2021).

### SNV/Indel CNV and Ploidy Calling

Data preprocessing. The raw sequencing reads were quality controlled by removing the adapters' bases and the low-quality bases (Phred-value <10) from the read-ends and by discarding the reads with 10% more ambiguous bases inside using Trimmomatic1 version 0.36. Murine sequences from the 3T3-J2 feeder cells were filtered using Xenome2 version 1.0.1 with default parameters. The remaining reads were aligned to the human reference genome (UCSC hg19) using BWA3 (version 0.7.15-r11403) under mem model requiring map quality >=40 (Phred-value). PCR duplicates were removed using Picard tool (version 2.15.0; https://broadinstitute.github.io/picard/). GATK(version 3.8.04) was used to realign the reads near indels (Mills_and_1000G_gold_standard indels bundled within GATK pipeline) and to recalibrate the base qualities with default settings following the best practice protocol.

SNVs/Indels calling. SNVs and Indels were called by Manta (version 1.3.25) and Strelka (version 2.9.26) with default parameter values in somatic calling model. Only SNVs/Indels which passed the default filter of Manta and Strelka in derived vcf files were used in downstream analyses. We also applied harder filters to the variant that require only two genotypes present, variant quality (Phred-value)>30, total read depth >15, alternative allele depth >5, and alternative allele proportion >5%. Also, we required that the corresponding matched normal sample should be homozygous wild type at the mutation sites. Somatic mutations were further filtered to remove possible germline mutations based on a panel of 27 normal samples. Somatic mutations with allele frequencies larger than 0.01 in 1000 Genome database or gnomAD database were discarded as well. SNVs and Indels were annotated with ANNOVAR web version.

CNV calling. The GATK9 somatic copy number variants calling pipeline version 4.0.4.0 (https://gatkforums.broadinstitute.org/gatk/discussion/9143/) was used to call the CNVs. We used 17 normal female samples sequenced on the same platform to build the CNV panel of normals (PoN) with extra parameter “--minimum-interval-median-percentile 10.0”. The contigs shorter then 46709983 bp were excluded from further analysis. The 1000G phase1 high-quality SNPs (1000G_phase1.snps.high-confidence bundled within GATK pipeline) was used to collect allelic counts information. In the segmentation step, we used patient-matched normal samples and applied parameters “--number-of-smoothing-iterations-per-fit 1--minimum-total-allele-count 15--window-size 7500”. Other steps used the default settings. Segments with less than 15 SNVs were excluded. After getting the segmented confidence interval of copy ratio and allele fraction information, the absolute allelic copy number was inferred and curated manually by considering the consistence of copy ratio and allele fractions and the unique features in different copy number (CN) patterns (e.g CN1's allele fraction=0 or 1, CN3's allele fraction=0.33 or 0.66). The CNV calling was confirmed using the ABSOLUTE (Carter et al., 2012) algorithm with default parameters. In selecting the best model of ABSOLUTE output, we required that each copy number peak should be under an integral number, the bottom peak for copy number should be close to zero, and the ploidy value should be very close (>0.95) to 1 because the data were obtained from single cell-derived clones.

### Phylogenetic Tree Construction and Ordering of Somatic Mutations

Ternary genotypes of filter-passed somatic SNVs identified from all WES data by Strelka were used in phylogenetic tree construction. The genotypes of normal sample (e.g. matched blood or fibroblast) were added as an outgroup. The trees were built by SiFit11 that employs a heuristic search algorithm to infer the Maximum Likelihood (ML) phylogenetic tree under a finite-site model of evolution. The number of iterations was set to 10000. The “InferAncestralStates” program of SiFit was used for inferring the order of somatic mutations on the branches of the phylogeny based on the false-negative rate, deletion rate, and LOH rate reported by SiFit during learning the tree in tree building step.

### Clonality Analysis

To ensure that each clone was derived from single cells, we analyzed the distribution of variant allele fractions (VAFs) of the identified somatic mutations in each sample. For monoclonal pedigrees, diploidy (2n) pedigrees' VAFs should distribute around 50% and triploidy (3n) pedigrees will have VAFs around 0.33 and 0.66. For polyclonal pedigrees, most of somatic mutations should have VAFs less than 0.5.

### Transcriptomic Sequencing Data Analysis

All RNA-seq libraries were constructed using the non-stranded kit from NEB (NEBNext Ultra II non-directional) and sequenced on Illumina NovaSeq 6000 platform with 150 bp pair-end reads. Raw reads were trimmed to remove low quality bases (phred score <20) and sequencing adapter leftovers by using Trim Galore (https://www.bioinformatics.babraham.ac.uk/projects/trim_galore/). Potential murine genomic DNA contaminant reads were removed for further analysis using Xenome. The remaining reads were aligned to the human reference genome (UCSC hg19) using Salmon (version 0.9.1) with default settings (61). Alignment results were then input to DEseq2 for differential expression analysis with default settings and FDR less than 0.05. The heatmaps with hierarchical clustering analysis of the global gene expression pattern in different samples were performed using pheatmap package (https://cran.rproject.org/web/packages/pheatmap/index.html) in R (version 3.5.1). The pathway enrichment analysis was performed using Enrichr (62).

### Statistical Analysis

Unpaired two-tailed student's t-test was used to determine the statistical significance between two groups. Statistical analyses were performed using R (version 3.5.1). The “n” numbers for each experiment are provided in the text and figures. P<0.05 was considered statistically significant. Asterisks denote corresponding statistical significance *p<0.05; **p<0.01; ***p<0.001 and ****p<0.0001.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than the specific embodiments that have been represented by way of examples.

### Example 7: Exemplary Synthesis Schemes

### Synthesis of 7532N (I-1)

A novel synthesis of the symmetric squaramide 7532N is described. The intermediate, (1S, 2R)-cis-1-(2-(2-azidoethoxy)ethoxy)-2-indanol (Compound 1), was prepared by O-alkylation of (1S, 2R)-cis-1-amino-2-indanol with the tosylate of 2-(2-azidoethoxy)ethan-1-ol. The tripeptide intermediate 7532P1 was synthesized via a four-step sequence involving the coupling of N-Boc-L-Chg-OH with H-L-Pro-OBzl using HATU and DIEA, followed by Boc deprotection, coupling with N-Boc-N-Me-L-Ala-OH, and catalytic hydrogenation to remove the benzyl ester. Intermediates 1 and 7532P1 were coupled, and the resulting azide (Compound 2) was reduced to the primary amine, which was subsequently conjugated with diethyl squarate to afford the symmetric squaramide. Finally, removal of the Boc protective groups using 12% TFA in DCM yielded the target compound 7532N. This concise synthesis provides an efficient route to a novel symmetric squaramide with potential biological applications.

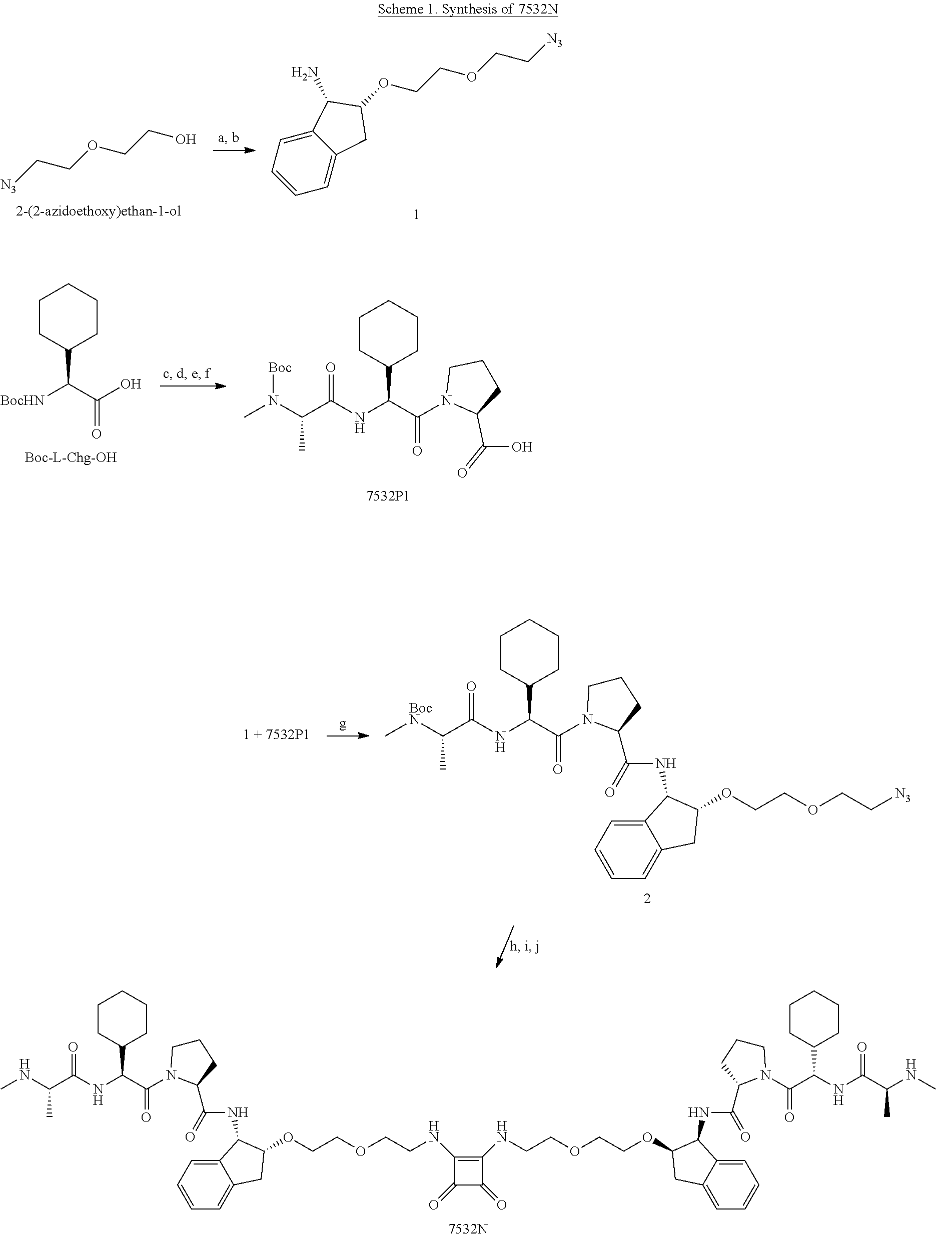

Scheme 1. Synthesis of 7532N. Reagents and Conditions: (a) p-TsCl, TEA, CH2Cl2; (b) NaH, (1S, 2R)-cis-1-amino-2-indanol, THF, 47% over two steps; (c) H-L-Pro-OBzl·HCl, HATU, DIEA, DMF; (d) 4N HCl in dioxane; (e) Boc-N-Me-L-Ala-OH, HATU, DIEA, DMF; (f) H₂, Pd/C, MeOH, quantitative yield over four steps; (g) HATU, DIEA, DMF, 85%; (h) H₂, Pd/C, MeOH; (i) 3,4-Diethoxy-3-cyclobutene-1,2-dione; (j) 12% TFA in DCM, 86% over the last two steps.

### Experimental Section

General Procedures. All solvents and organic reagents were obtained from commercial sources and used without further purification unless otherwise stated. Semi-preparative reversed-phase high-performance liquid chromatography (RP-HPLC) was performed on a Varian semi-preparative system equipped with a Discovery C18 569226—U RP-HPLC column (25 cm×21.2 mm, 5 μm). The mobile phase consisted of a gradient of water (0.1% TFA) and acetonitrile, with a flow rate of 20 mL/min. Mass spectra and HPLC retention times were obtained using a Thermo LTQXL LC/MS system with a UV detector (monitoring at 215 and 254 nm) and an Agilent 300SB-C8 RP-HPLC column (4.6×100 mm, 3.5 μm). The mobile phase consisted of a gradient of A) water (0.1% TFA) and B) acetonitrile at a flow rate of 0.5 mL/min. Unless otherwise specified, all HPLC retention times are reported for an eluent gradient of 5% B for the first 3 min, followed by an increase from 5% to 98% B over 6 min, which was maintained for an additional 6 min. ¹H NMR spectra were recorded on a Bruker Avance 600 MHz spectrometer and are referenced according to the residual peak of the solvent based on literature values. Crude target compounds were purified by RP-HPLC using the Varian semi-preparative system described above.

### Synthesis of 2-(2-azidoethoxy)ethan-1-ol tosylate

To a solution of 2-(2-azidoethoxy)ethan-1-ol (1.8 g, 13.7 mmol) and triethylamine (2.25 mL, 16.1 mmol) in CH₂Cl₂ (10 mL) at 0° C. was added dropwise a solution of p-toluenesulfonyl chloride (2.15 g, 11.3 mmol) in CH₂Cl₂ (15 mL). The resulting suspension was stirred at room temperature overnight. Water (10 mL) was added and stirred for 20 min. The organic layer was separated, washed with 0.1 N KHSO₄ (2×10 mL) and brine, dried over Na₂SO₄, filtered and concentrated. The residue was purified by silica gel chromatography (0-10% MeOH/CH₂Cl₂) to give the tosylate product (3.0 g).

### Synthesis of Compound 1

To a suspension of NaH (60% in mineral oil; 0.9 g, 22.5 mmol) in anhydrous THF (60 mL) at 0° C. was added slowly a solution of (1S,2R)-cis-1-amino-2-indanol (3.0 g, 20 mmol) in THE (100 mL). The mixture was warmed to room temperature over 30 min and then heated to reflux. A solution of the tosylate from above (3.0 g, 10.5 mmol) in THE (10 mL) was added dropwise. The reaction was refluxed for 6 h, cooled, carefully quenched with water (30 mL), and concentrated to remove THF. The aqueous residue was extracted with CH₂Cl₂ (3×50 mL). The combined organic extracts were washed with brine, dried over Na₂SO₄, filtered, and concentrated. The crude product was purified by silica gel chromatography (0-10% MeOH/CH₂Cl₂) to give compound 1 (1.4 g, 47% over 2 steps). LC-MS (ESI+) m/z: 263.22 [M+H]⁺; t_{R}=7.93 min.

### Synthesis of Compound 7532P1

N-Boc-L-Chg-OH (10.29 g, 40 mmol), HATU (16 g, 42 mmol), DIEA (15.2 mL, 92 mmol), and L-Pro-OBzl·HCl (10.2 g, 42 mmol) were combined in anhydrous DMF (200 mL) at 0° C. The mixture was stirred at room temperature for 2 h and concentrated. The residue was partitioned between EtOAc (600 mL) and 0.25 N KHSO₄ (3×100 mL), saturated NaHCO₃ (3×100 mL), and brine (3×100 mL). The organic layer was dried over MgSO₄, filtered, and concentrated to an oil. This was treated with 4 N HCl in dioxane (200 mL) at room temperature for 2 h. Concentration and co-evaporation with CH₂Cl₂ (3×100 mL) provided H-L-Chg-L-Pro-OBzl·HCl (22.1 g) as an oil.

N-Boc-N-Me-L-Ala-OH (7.74 g, 38 mmol) was coupled to the H-L-Chg-L-Pro-OBzl·HCl (15.3 g, 40 mmol) using HATU (15.2 g, 40 mmol) and DIEA (14.5 mL, 87.6 mmol) in DMF (200 mL) as described above. Purification by silica gel chromatography (25:1 CH₂Cl₂/MeOH) gave the protected tripeptide (16.6 g). This was hydrogenated over 10% Pd/C (2.0 g) in MeOH (200 mL) under a H₂ balloon overnight. Filtration through Celite and concentration gave a residue that was triturated with hexane (200 mL). Decantation and drying provided 7532P1 (12.8 g, quantitative) as a white powder. LC-MS (ESI+) m/z: 901.63 [2M+Na]⁺, 462.69 [M+Na]⁺, 440.53 [M+H]⁺; t_{R}=10.78 min.

### Synthesis of Compound 2

Compound 7532P1 (527 mg, 1.2 mmol), HATU (480 mg, 1.2 mmol), DIEA (0.48 mL), and compound 1 (0.4 g, 1.5 mmol) were combined in anhydrous DMF (6 mL) at 0° C. The mixture was stirred at room temperature for 2 h and concentrated. The residue was purified by preparative RP-HPLC (C18, 21.2×250 mm, 5 μm; 30-98% MeCN/0.1% aq. TFA over 15 min, 20 mL/min, 215 nm). Lyophilization of the product fractions gave compound 2 (0.7 g, 85%) as a white powder. LC-MS (ESI+) m/z: 684.27 [M+H]⁺; t_{R}=10.37 min.

### Synthesis of Compound 7532N

A solution of compound 2 (0.52 g, 0.76 mmol) in MeOH (20 mL) was hydrogenated over 10% Pd/C (60 mg) under a H₂ balloon for 3 h. Filtration through Celite and concentration gave a residue that was redissolved in anhydrous THF (9 mL) and treated with diethyl squarate (12 μL, 0.082 mmol) under Ar overnight. Concentration and purification by preparative RP-HPLC (C18, 21.2×250 mm, 5 μm; 40-98% MeCN/0.1% aq. TFA over 20 min, 20 mL/min, 215 nm) afforded the Boc-protected intermediate after lyophilization. This was treated with TFA (1.2 mL) in CH₂Cl₂ (9 mL) at 0° C. and stirred at room temperature for 2 h. Concentration and purification by preparative RP-HPLC (C18, 21.2×250 mm, 5 μm; 10-98% MeCN/0.1% aq. TFA over 20 min, 20 mL/min, 215 nm) gave 7532N (100 mg, 86% over 2 steps) as a white powder after lyophilization. ¹H NMR (600 MHz, D₂O) δ 7.33-7.28 (m, 4H), 5.32 (d, J=5.4 Hz, 1H), 4.48-4.34 (m, 3H), 4.03-3.90 (m, 2H), 3.75-3.65 (m, 8H), 3.10-2.99 (m, 2H), 2.70 (s, 3H), 2.30-2.25 (m, 1H), 2.15-1.95 (m, 3H), 1.83-1.64 (m, 6H), 1.51 (d, J=7.2 Hz, 3H), 1.25-1.07 (m, 6H). ¹³C NMR (151 MHz, DMSO-d6) δ 182.48, 171.39, 169.17, 168.48, 167.74, 158.17, 157.91 (TFA), 141.68, 140.23, 127.58, 126.37, 124.74, 124.32, 80.74, 70.17, 69.69, 68.68, 59.23, 56.04, 55.82, 55.42, 55.31, 47.34, 43.06, 36.38, 30.74, 29.10, 28.57, 28.24, 25.80, 25.68, 25.54, 24.63, 15.68. LC-MS (ESI+) m/z: 1194.06 [M+H]⁺, 598.08; t_{R}=8.63 min.

### Example 8: Efficacy of Compositions Described Herein on Various Tumor Models

The present example describes methods used to test the efficacy of compounds, compositions, and/or combinations described herein in the treatment of various tumors in mouse human tumor xenograft model. More, specifically the present example describes methods used to evaluate the in vivo antitumor efficacy of the combination treatment I-1 (7532N) and ponatinib (121-H) across a panel of 27 patient-derived cancer xenograft models in single mouse trial (SMT) format. The study consisted of 27 efficacy experiments with 27 tumor models implanted subcutaneously in female NSG mice in a single mouse trial format. A listing of tumor types, experiment numbers, and experiment duration used for the studies described herein are provided in Table 2.

Each experiment was set up with 2 groups consisting of one mouse each. One group/mouse was given 7532-N and 121-H in combination, administered orally and separately twice weekly for up to three weeks. The second mouse received the vehicles for both test articles as a control for reference. The duration of each experiment is given in Table 2. The overall design of each experiment is given in Table 3

TABLE 2

| | | | Final | |
|---|---|---|---|---|
| Tumor | Tumor | Experiment | Dosing | Last Day of |
| Type | Model | No. | Day* | Experiment |
| Gastric | GXA 3002 | V1314 | 11/17 | 25 |
| cancer | GXA 3011 | V1300 | 21 | 25 |
| | GXA 3012 | V1312 | 21 | 25 |
| | GXA 3023 | V1301 | 21 | 24 |
| | GXA 3027 | V1298 | 21 | 32 |
| | GXA 3037 | V1302 | 21 | 24 |
| | GXA 3039 | V1315 | 21/9 | 25 |
| | GXA 3063 | V1310 | 21 | 24 |
| | GXA 3087 | V1316 | 15/13 | 18 |
| | GXF 602 | V1295 | 13 | 25 |
| Ovarian | OVXF 550 | V1296 | 9/13 | 32 |
| cancer | OVXF 899 | V1297 | 21 | 22 |
| | OVXF | V1317 | 21 | 24 |
| | 1320 | | | |
| | OVXF | V1308 | 21 | 22 |
| | 1353 | | | |
| | OVXF | V1299 | 21 | 22 |
| | 1993 | | | |
| | OVXF | V1340 | 21 | 25 |
| | 2437 | | | |
| | OVXF | V1311 | 21 | 25 |
| | GYN090 | | | |
| Pancreatic | PAXF 736 | V1307 | 21 | 29 |
| cancer | PAXF 1657 | V1303 | 21 | 25 |
| | PAXF 2005 | V1318 | 21/9 | 25 |
| | PAXF 2035 | V1319 | 21 | 25 |
| | PAXF 2059 | V1320 | 21 | 25 |
| | PAXF 2082 | V1194 | 21 | 25 |
| | PAXF 2094 | V1304 | 21 | 25 |
| | PAXF 2116 | V1305 | 21 | 29 |
| | PAXF 2146 | V1384 | 21 | 25 |
| | PAXF 2196 | V1306 | 17 | 22 |

*When 2 final dosing days are given, these apply to Groups 1 and 2 respectively.

TABLE 3

| | | Total Daily | | | No. |
|---|---|---|---|---|---|
| Group | Test | Dose | Schedule | | of |
| ID | Article¹ | [mg/kg/day] | [Dosing days]² | Route | Animals |
| 1 | Control Vehicle// | 5 ml/kg// | 1, 3, 5, 7, 9, 11, 13, 15, 17, 19, 21// | p.o.// | 1 |
| | Control Vehicle | 5 ml/kg | 1, 3, 5, 7, 9, 11, 13, 15, 17, 19, 21 | p.o. | |
| 2 | 7532-N// | 5//30 | 1, 3, 5, 7, 9, 11, 13, 15, 17, 19, 21// | p.o.// | 1 |
| | 121-H | | 1, 3, 5, 7, 9, 11, 13, 15, 17, 19, 21 | p.o. | |

¹Vehicle for 7532-N: 1% DMSO, 39.6% PEG300, 4.95% Tween 80 in PBS; vehicle for 121-H: 6% DMSO, 37.6% PEG300, 4.7% Tween 80 in PBS
²The dosing schedule was shorter in 7 experiments.

Antitumor efficacy of the test group was assessed using the vehicle control group as a reference. Tumor growth inhibition was determined by the comparison of relative tumor volume (RTV) of the test group with the vehicle control group and is expressed as minimum test versus control (T/C) value in percent. Tumor samples were collected at the end of each experiment and were processed as detailed in Table 4.

TABLE 4

| | Type of | | |
|---|---|---|---|
| Group | Sample, | Time After Last | Sample |
| ID | Fixation | Treatment | Amount |
| 1, 2 | Tumor ½ FFPE, ½ SF | 24-96 h after final dose | Half each |

Formulation: Vehicle for 7532-N: 1% DMSO, 39.6% PEG300, 4.95% Tween 80 in PBS; vehicle for 121-H: 6% DMSO, 37.6% PEG300, 4.7% Tween 80 in PBS. Vehicle for 121-H: 6% DMSO, 37.6% PEG300, 4.7% Tween 80 in PBS.

Animals were monitored until the tumor implants reached the study volume criteria of 50-75 mm³, in a sufficient number of animals. Mice were assigned to groups aiming at comparable tumor volumes. The day of randomization was designated as Day 0 of the experiment. The percentage of all tumor implants suitable for randomization at the standard volume of 50-250 mm³ is defined as the take rate according to the following equation:

$$Take Rate [ \% ] \frac{= number of tumors suitable for randomization}{total number of implanted fragments} \times 100$$

Take rates of PDX at standard randomization criteria in NMRI nu/nu mice are routinely recorded and a median take rate is calculated for characterization purposes. The time from implantation to randomization at a standard tumor volume is expressed in days as “Induction time (IT)”. Induction times of tumors are routinely recorded, and a median IT is calculated for characterization purposes.

Animals were weighed twice a week, or daily if body weight loss in excess of 10% was recorded. Relative body weights of individual animals were calculated by dividing the individual body weight on Day x (BWₓ) by the individual body weight on the day of randomization (BW₀) multiplied by 100:

$$R B {W}_{x} [ \% ] = \frac{B {W}_{x} [ g ]}{B {W}_{0} [ g ]} \times 100$$

Relative body weights (RBW) were calculated for evaluation purposes. Individual body weight changes in % were calculated by dividing the body weight change from the day of randomization to Dayₓ (BWₓ−BWₒ) by the body weight on the day of randomization (BWₒ) multiplied by 100.

$$Body weight change ( {Day}_{x} ) [ \% ] = \frac{B {W}_{x} - B {W}_{0}}{B {W}_{0}} \times 100$$

The absolute tumor volumes (ATVs) given in Appendix 1 were determined by two-dimensional measurement with a digital caliper (S_Cal EVO Bluetooth, Switzerland) on the day of randomization and then twice weekly. Tumor volumes were calculated according to the formula

$$Tumor volume = ( 1 \times {w}^{2} ) \times 0.5$$

where l=largest diameter and w=width (perpendicular diameter) of the tumor (in mm). Relative volumes of individual tumors (individual RTVs) for Day x were calculated by dividing the absolute individual tumor volume on Day x (Tₓ) by the absolute individual tumor volume of the same tumor on the day of randomization (Tₒ) multiplied by 100:

$$R T {V}_{x} [ \% ] = \frac{{T}_{x}}{{T}_{0}} \times 100$$

RTV values were used for drawing growth curves and for treatment evaluation.

Tumors were collected immediately after euthanasia, divided and directly transferred to liquid nitrogen (snap-frozen samples) or fixative (FFPE samples). The fixation was performed in 10% neutral buffered formalin for approximately 24 h. The fixative was then replaced by submerging the samples in 70% ethanol for up to seven days. Thereafter, samples were dehydrated by sequentially incubating them in the following solutions: 70% ethanol (two times 0.5 h), 80% ethanol (two times 1 h), 100% ethanol (two times 0.5 h), 100% isopropanol (1.5 h), xylene (two times: 1 h; 1.5 h). Finally, samples were infiltrated by and embedded in paraffin. Tumor samples were not collected in cases of complete tumor remission or severe ulceration at the tumor site.

### Data Evaluation

### Survival Rate

The survival rate (Table 7) was calculated by counting the number of animals that would have survived beyond the last experimental day of each group and dividing them by the total number of animals in the group. Animals that died or were euthanized on the last day of the group for any other reason than sample collection or termination of the group were not counted as survivors. The adjusted survival rate in Table 7 was calculated by counting all surviving animals including those that were euthanized for tumor-related reasons and dividing them by the total number of animals in the group. The following reasons for euthanasia are classed as tumor-related: 1) tumors fulfilling volume-related euthanasia criteria including accessory tumors, 2) ulcerating tumors and 3) dermal necrosis at the tumor site. Euthanasia of animals due to symptoms of tumor-induced cachexia is not counted as tumor-related.

### Tumor Volume and Growth

Tumor volume doubling and quadrupling time (Td, Tq) for test and control groups is defined as the time interval (in days) required for a group to reach a median RTV of 200% or 400%. Data are presented in Table 6. The test versus control value (T/C in %) was calculated from the ratio of the RTV value of test versus control group on Day x multiplied by 100.

$$T / C [ \% ] = \frac{R T {V}_{x} treated group}{R T {V}_{x} control group} \times 100$$

The minimum T/C value recorded for a test group during an experiment represents the maximum antitumor efficacy for the respective treatment.
Group minimum T/C values were used for efficacy rating as follows:

| | | |
|---|---|---|
| − | Inactive | T/C > 65% |
| +/− | Borderline efficacy | 50% ≤ T/C ≤ 65% |
| + | Moderate efficacy | 25% ≤ T/C < 50% |
| ++ | High efficacy | 10% ≤ T/C < 25% |
| +++ | Very high efficacy | 5% ≤ T/C < 10% |
| ++++ | Regression | T/C < 5% |

In addition, group tumour growth was evaluated according to the response evaluation criteria in solid tumours (RECIST) modified for preclinical in vivo efficacy studies. The tumour growth response (R in %) was calculated from the difference of the mean ATV values on Day x (ATVₓ) to Day 0 (ATV₀) divided by the mean ATV₀ multiplied by 100 as follows:

$$Response [ \% ] = \frac{Mean {ATV}_{x} - Mean {ATV}_{0}}{Mean {ATV}_{0}} \times 100$$

| | |
|---|---|
| Complete Response (CR) | R = −100% |
| Partial Response (PR) | −100% < R < −50% |
| Stable Disease (SD) | −50% ≤ R < +35% |
| Progressive disease (PD) | R > +35% |

Group tumour response (R) values were assessed at the end of individual groups and are documented in Table 6.

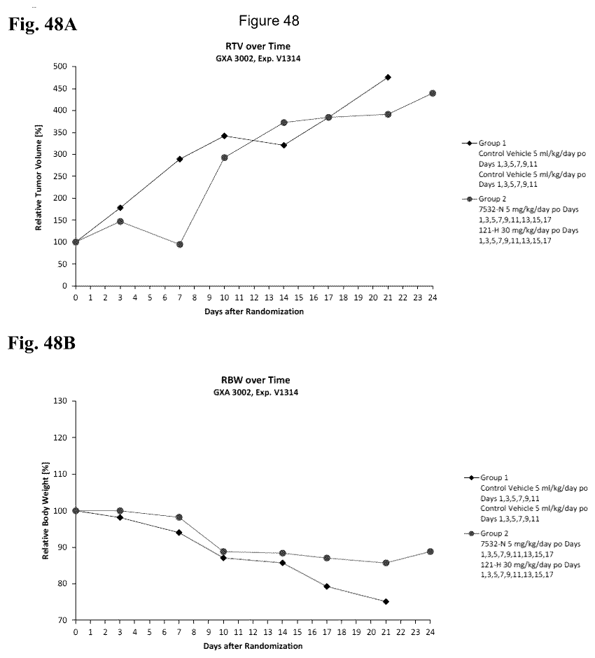

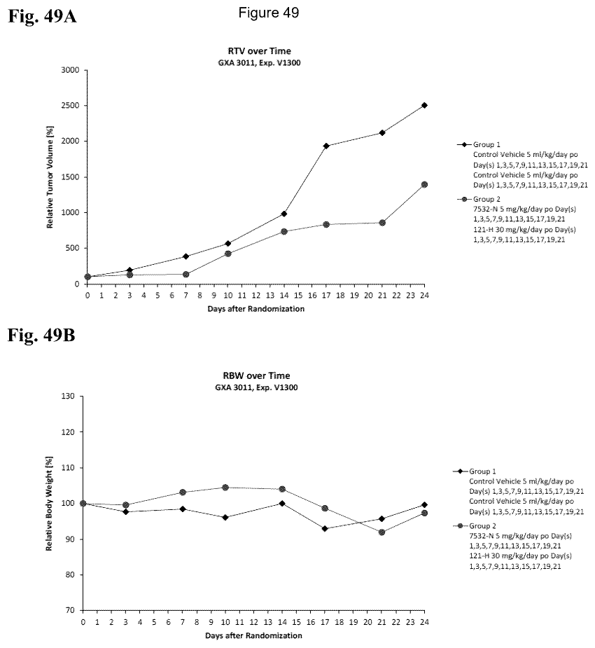

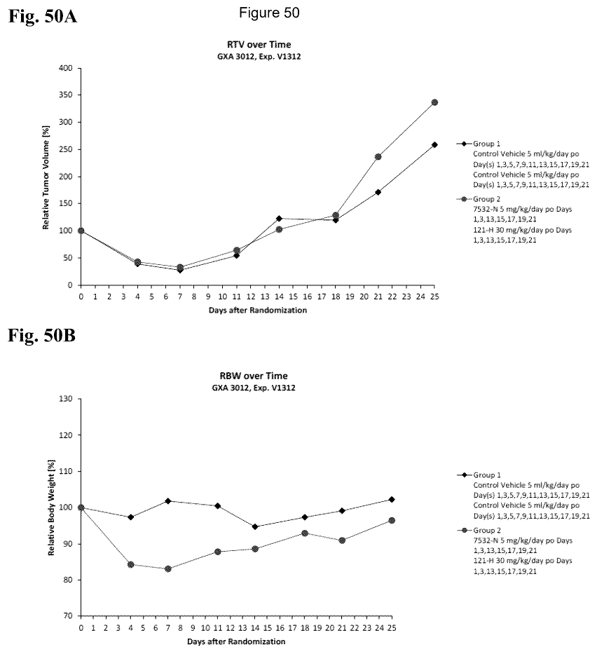

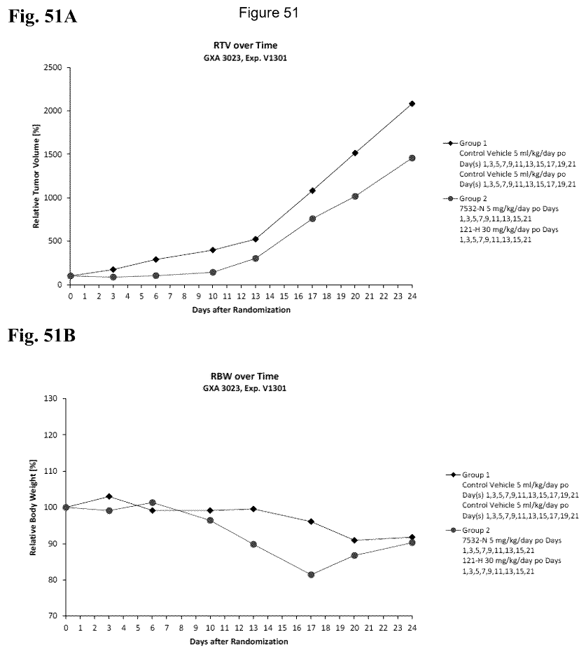

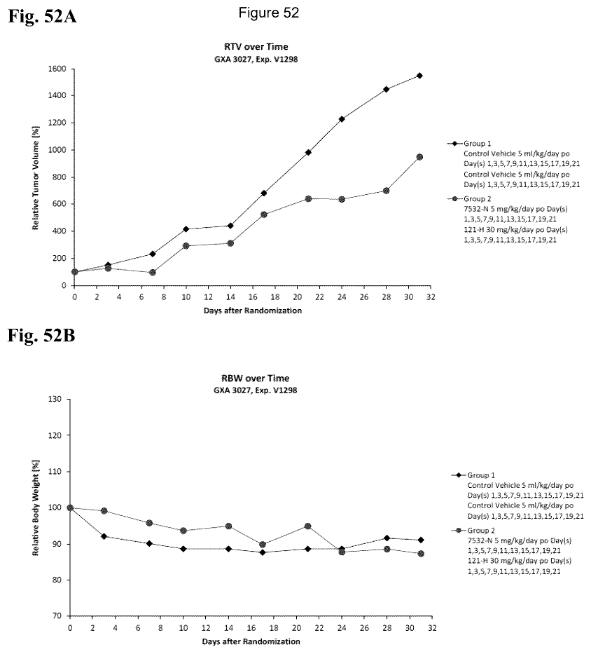

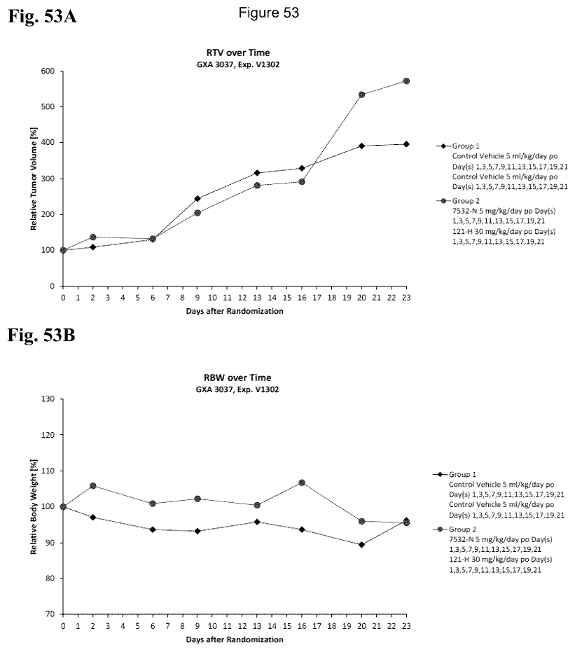

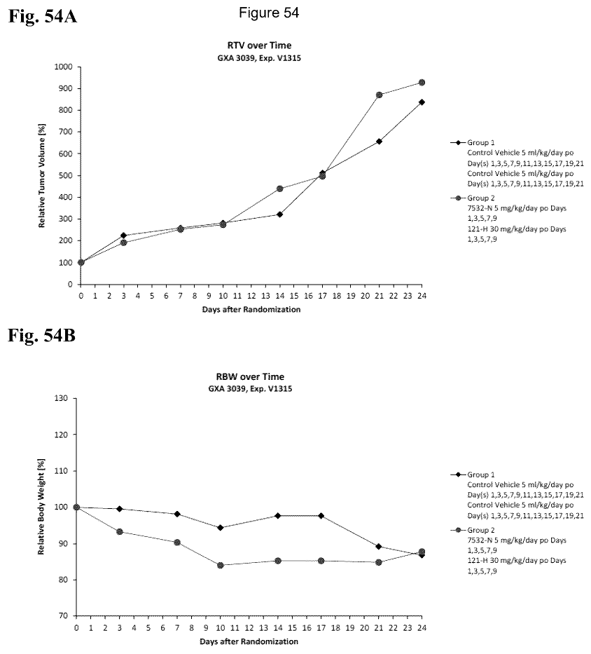

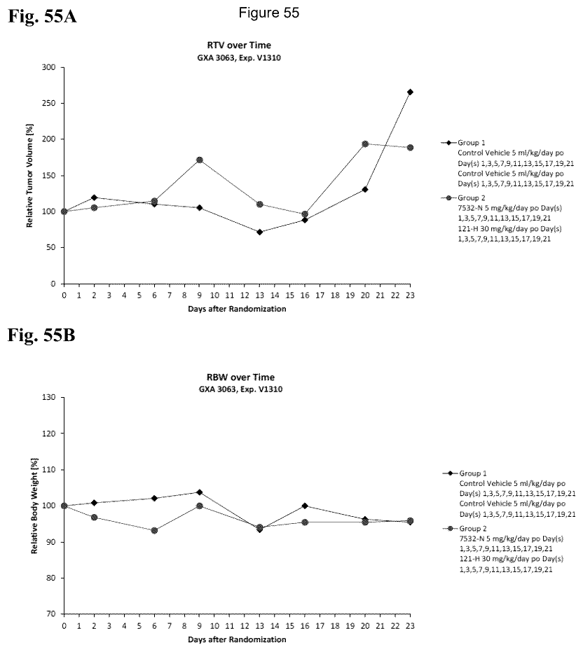

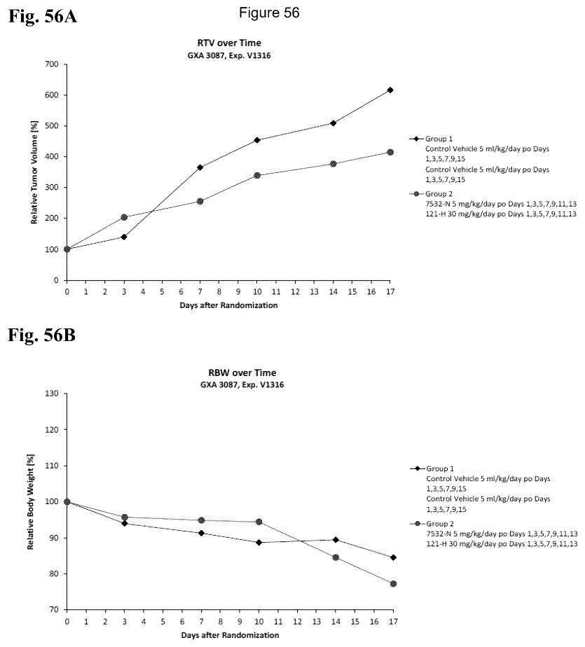

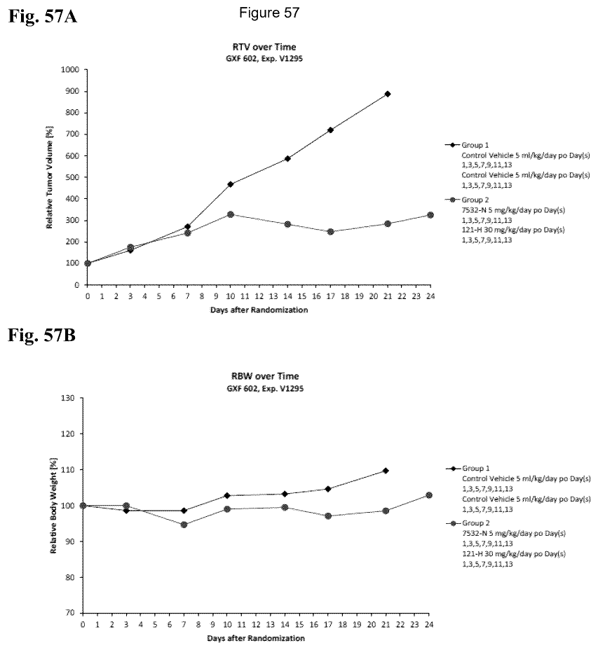

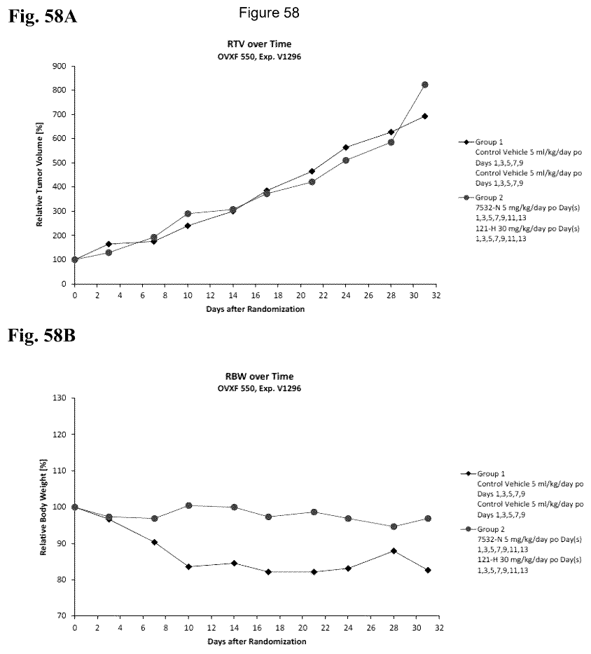

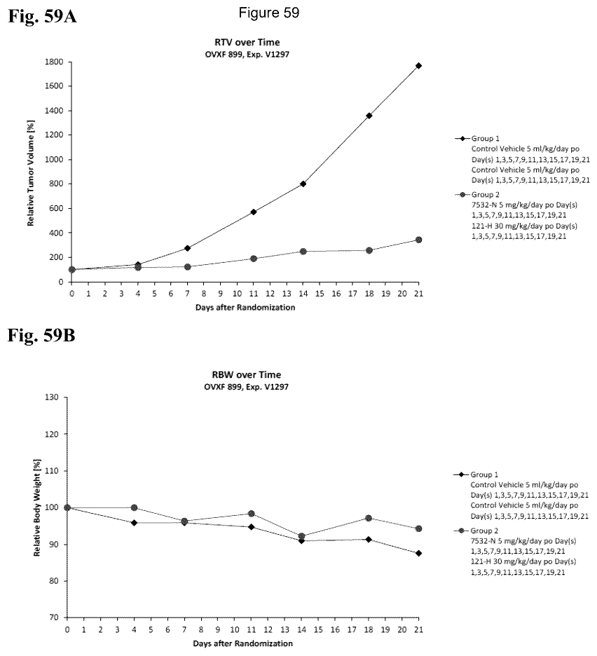

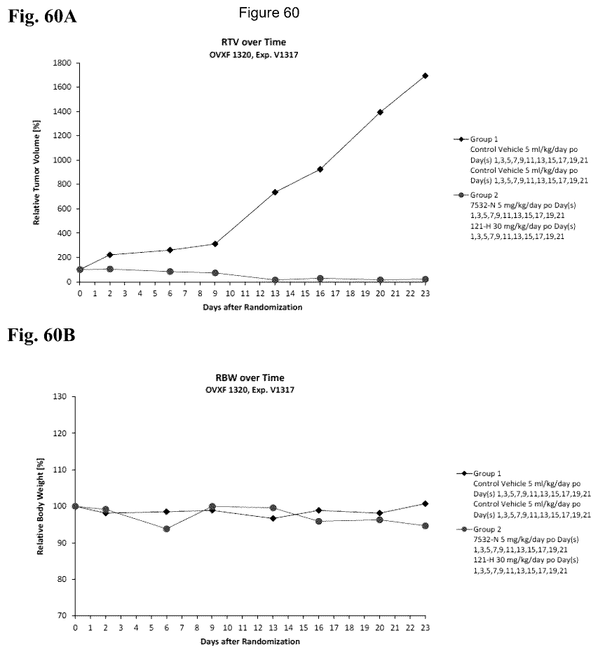

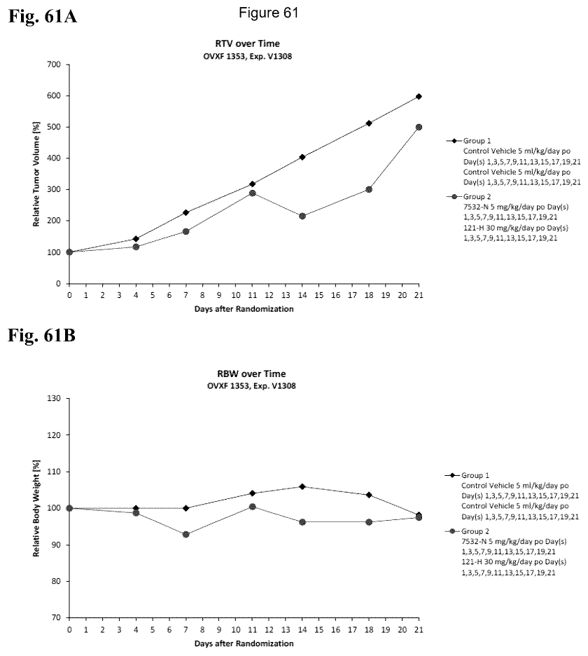

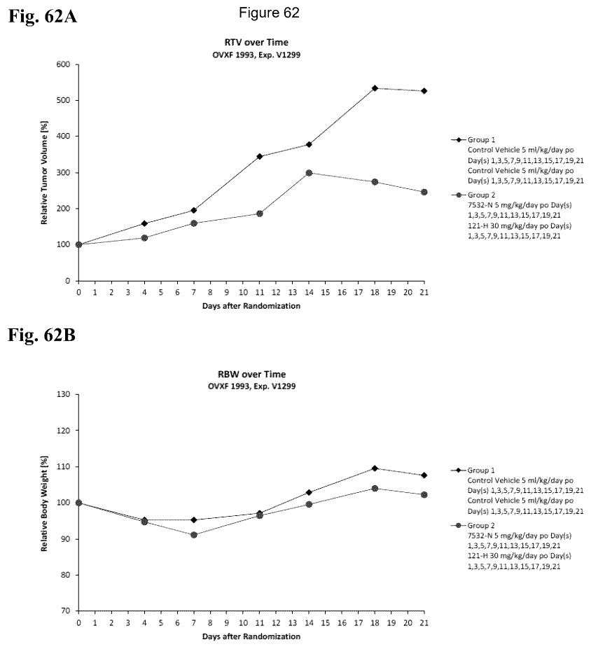

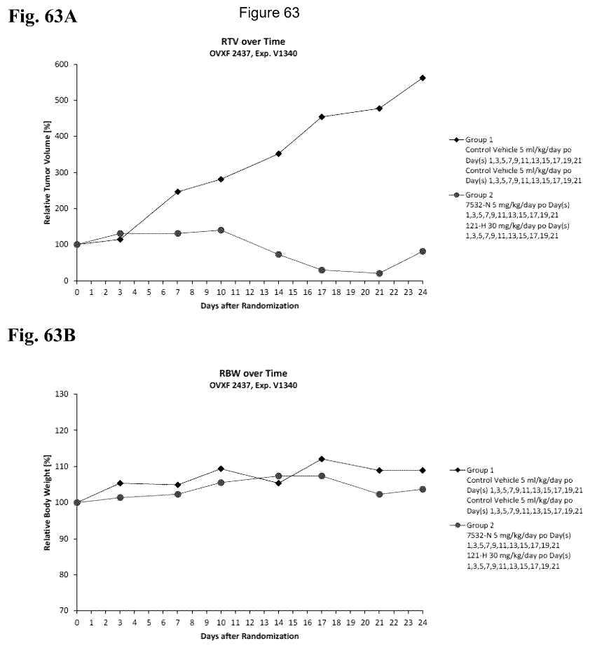

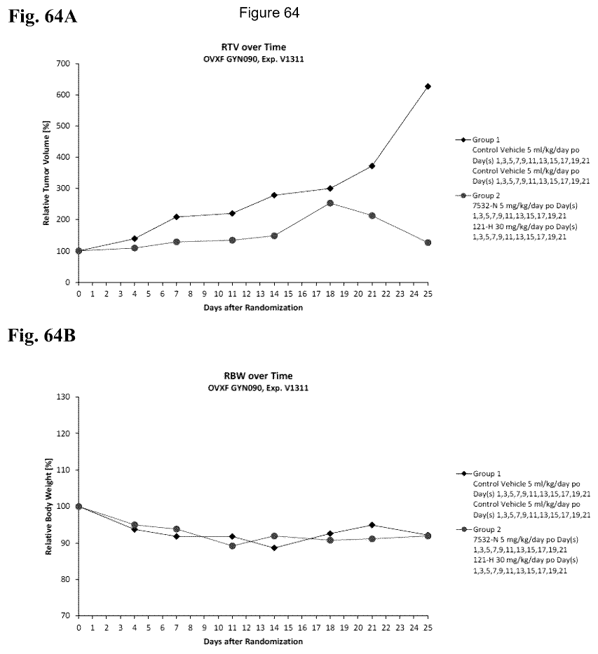

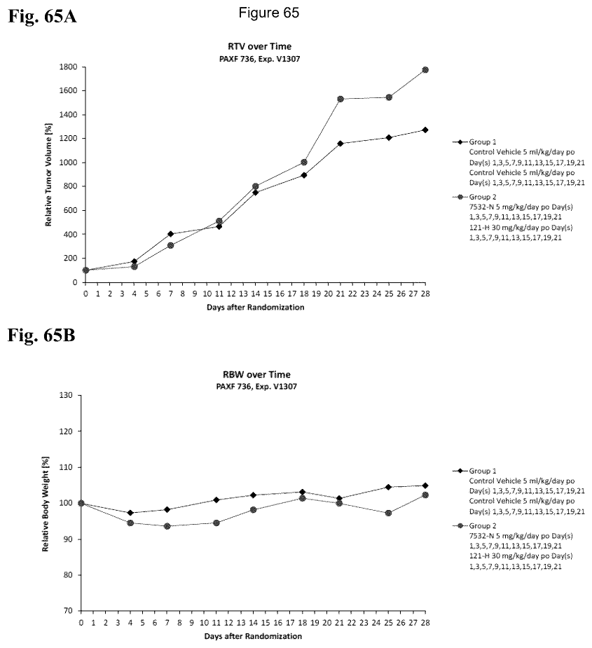

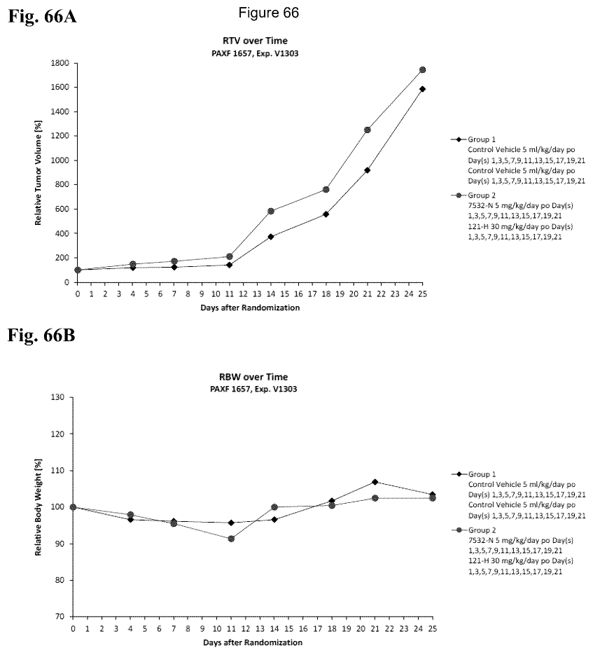

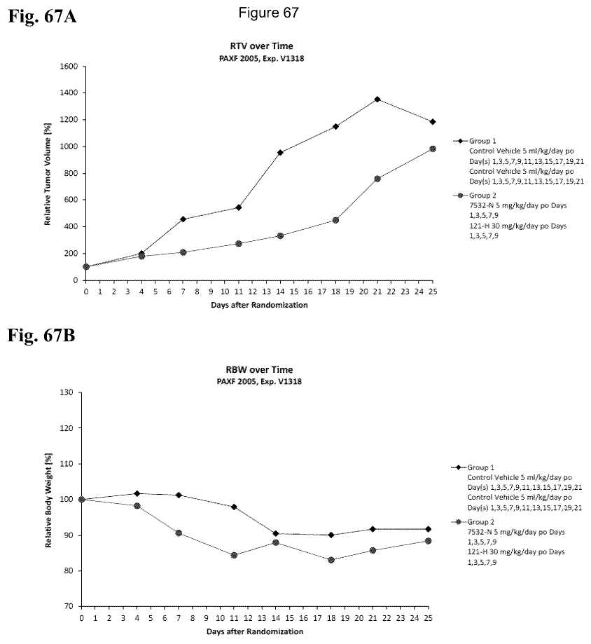

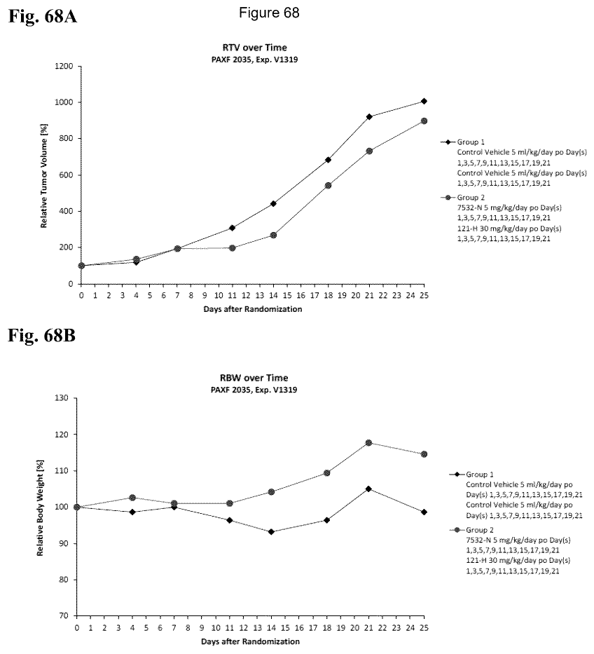

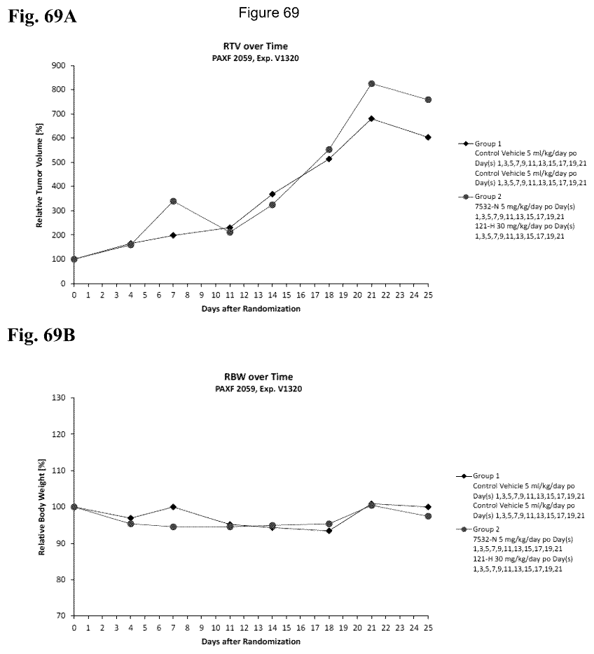

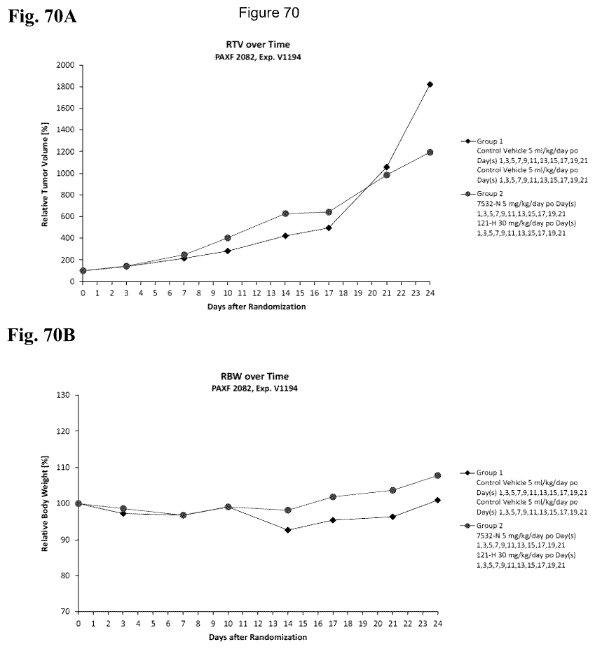

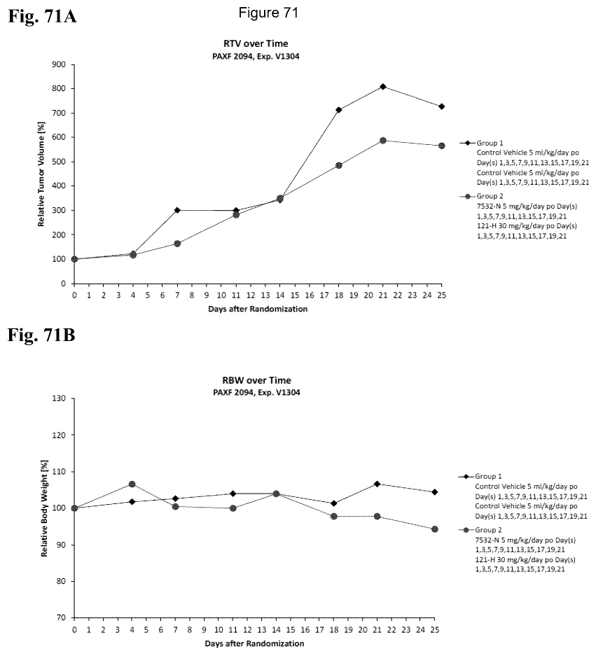

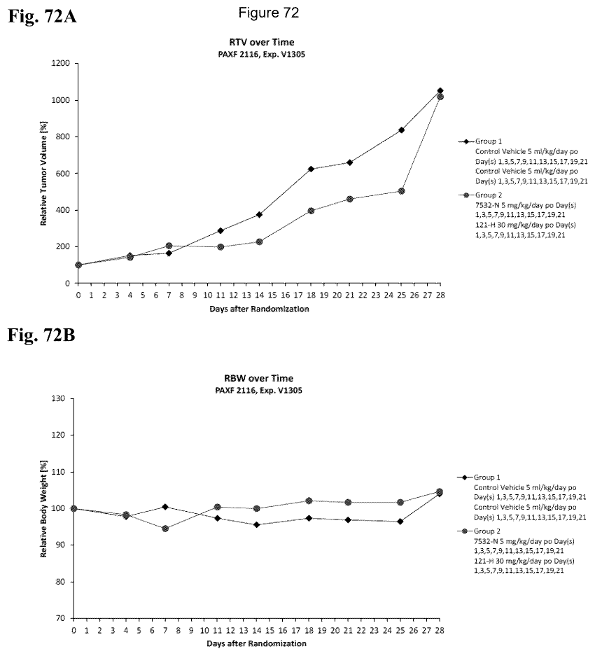

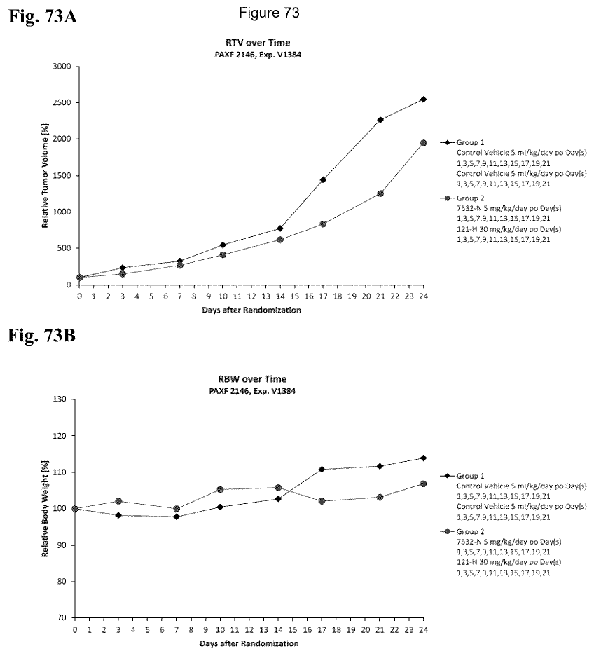

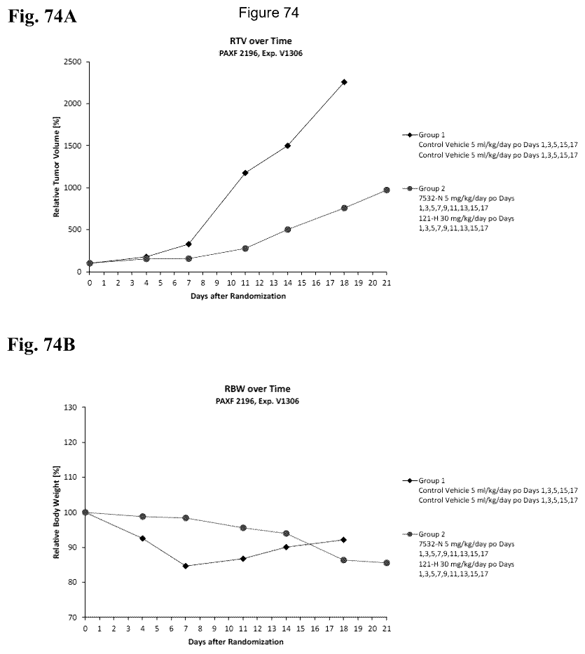

The data presented demonstrate that the combinations described herein are efficacious in the treatment of various cancers. Specifically, using human tumour patient xenografts of gastric cancer, ovarian cancer, and pancreatic cancer, the data demonstrates that for various cancer types treatment with the combination described herein slowed both tumour growth and weight loss relative to control treatment (see FIGS. 48-74).

TABLE 5
Characteristics of the Human Tumor Xenografts

| | | | Pre- | | | | | | |
|---|---|---|---|---|---|---|---|---|---|
| | | | Implantation | | | Age | | | |
| Tumor | Tumor | Histology | Chemo/ | | | (Gender) of | TR | IT | Td |
| Type | Designation | Differentiation | Radiotherapy | Origin | Stage¹ | Patient | [%] | [Days] | [Days] |
| Gastric | GXA 3002 | Adenocarcinoma Lauren | None | Primary | Not available | 80 (Female) | 78 | 21 | 6.6 |
| cancer | | intestinal type/not known | | | | | | | |
| | GXA 3011 | Adenosquamous | None | Primary | Not available | 63 (Male) | 81 | 15 | 3.9 |
| | | carcinoma/poor | | | | | | | |
| | GXA 3012 | Adenocarcinoma Lauren | None | Primary | Not available | 65 (Male) | 34 | 41 | 10.1 |
| | | diffuse/poor | | | | | | | |
| | GXA 3023 | Adenocarcinoma Lauren | None | Primary | Not available | 72 (Female) | 81 | 19 | 5.3 |
| | | intestinal type/moderate | | | | | | | |
| | GXA 3027 | Adenocarcinoma Lauren | None | Primary | Not available | 57 (Male) | 75 | 27 | 5.7 |
| | | intestinal type/poor | | | | | | | |
| | GXA 3037 | Adenocarcinoma Lauren | None | Primary | Not available | 69 (Male) | 56 | 30 | 9.6 |
| | | intestinal type/moderate | | | | | | | |
| | GXA 3039 | Adenocarcinoma Lauren | None | Primary | Not available | 65 (Male) | 51 | 27 | 6.0 |
| | | intestinal type/moderate | | | | | | | |
| | GXA 3063 | Adenocarcinoma/poor | None | Primary | AGC Borrmann type III | 84 (Male) | 82 | 28 | 5.3 |
| | | | | | T3 or T4a | | | | |
| | GXA 3087 | Adenocarcinoma/poor | None | Primary | Not available | 58 (Female) | 60 | 28 | 1.4 |
| | GXF 602 | Adenocarcinoma/poor | None | Primary | TxN1M0 | 71 (Female) | 67 | 21 | 5.6 |
| Ovarian | OVXF 550 | Serous adenocarcinoma/ | Not known | Primary | Not available | (Female) | 83 | 26 | 8.6 |
| cancer | | poor | | | | | | | |
| | OVXF 899 | Serous adenocarcinoma/ | Radiation | Primary | T3N0M0 | 76 (Female) | 66 | 24 | 7.3 |
| | | good | | | | | | | |
| | OVXF 1320 | Serous adenocarcinoma/ | Not known | Primary | T3NxMx | 79 (Female) | 49 | 78 | 14.4 |
| | | moderate | | | | | | | |
| | OVXF 1353 | Serous adenocarcinoma/ | Not known | Metastasis | N1 | 41 (Female) | 76 | 26 | 6.4 |
| | | poor | | | | | | | |
| | OVXF 1993 | Serous adenocarcinoma/ | Taxol/Carbo | Primary | Not available | 78 (Female) | 69 | 32 | 9.2 |
| | | not known | Gem | | | | | | |
| | OVXF 2437 | Serous adenocarcinoma/ | Not known | Metastasis | Not available | 83 (Female) | 72 | 55 | 17.0 |
| | | moderate | | | | | | | |
| | OVXF | Serous adenocarcinoma/ | Not known | Primary | Not available | (Female) | 33 ² | 14 ² | 6.6 ² |
| | GYN090 | moderate | | | | | | | |
| Pancreatic | PAXF 736 | Adenocarcinoma/poor | None | Recurrent | M1 | 65 (Male) | 78 | 23 | 5.2 |
| cancer | PAXF 1657 | Adenocarcinoma/ | GEM | Metastasis | M1 lung | 60 (Female) | 74 | 19 | 2.8 |
| | | moderate | | | | | | | |
| | PAXF 2005 | Adenocarcinoma/poor | None | Primary | pT3, pN1(4/17), pMx, L1, | 69 (Female) | 79 | 20 | 5.5 |
| | | | | | V0, Pn1 | | | | |
| | PAXF 2035 | Adenocarcinoma/ | None | Primary | pT3, pN1 (3/13), pMx pMx, | 71 (Female) | 78 | 28 | 5.5 |
| | | moderate | | | L1, Pn1, R0 | | | | |
| | PAXF 2059 | Invasive ductal | None | Primary | pT3, pN1 (2/5), cM0.L1. | 73 (Female) | 73 | 33 | 6.3 |
| | | carcinoma/poor | | | V0. Pn1. R1 | | | | |
| | PAXF 2082 | Adenocarcinoma/poor | None | Primary | pT3pN1, L1, V0, Pn1 | 74 (Male) | 75 | 28 | 5.8 |
| | PAXF 2094 | Adenocarcinoma/poor | None | Primary | pT3pN1.L0.Vx.Pn1 | 84 (Male) | 84 | 22 | 7.5 |
| | PAXF 2116 | Adenocarcinoma/poor | None | Primary | pT3pN1pM1(Hep)L1, V1Pn1 | 55 (Male) | 77 | 20 | 4.7 |
| | PAXF 2146 | Adenocarcinoma/ | None | Primary | pT3.pN1(1/24).L0V0; R0 | 88 (Male) | 67 | 30 | 7.4 |
| | | moderate | | | | | | | |
| | PAXF 2196 | Adenocarcinoma/not | Not known | Primary | Not available | (Female) | 59 | 20 | 3.4 |
| | | known | | | | | | | |

TR = tumor take rate (proportion of implanted tumors suitable for randomization); IT = induction time (time interval between tumor implantation and randomization/start of dosing); Td = tumor volume doubling time; Data are median values for vehicle control groups of experiments carried out at Charles River Laboratories Germany GmbH atstandard randomization criteria of tumor volumes of 50-250 mm³ in NMRI nu/nu mice.
¹Stage according to the TNM Staging Guide or according to the International Classification of Diseases for Oncology (ICD-O, issued by the World Health Organization)
² Data are from this study in NSG mice only.

TABLE 6
Antitumor Efficacy

| | | | | | | | | | | |
|---|---|---|---|---|---|---|---|---|---|---|
| | | Dose | | | Minimum | | | | | |
| Group | | Level [mg/ | | | T/C [%] | Efficacy | Td | Tq | R [%]³ | |
| ID | Treatment ¹ | kg/day] | Schedule [Day] | Route | (Day)² | Rating | [Days] | [Days] | (Day) | RECIST³ |

Tumor Model GXA 3002- Exp. V1314

| | | | | | | | | | | |
|---|---|---|---|---|---|---|---|---|---|---|
| 1 | Vehicle 1// | 5 ml/kg// | 1, 3, 5, 7, 9, 11// | p.o.// | n/a | n/a | 3.8 | 17.7 | 375.6 | PD |
| | Vehicle 2 | 5 ml/kg | 1, 3, 5, 7, 9, 11 | p.o | | | | | (21) | |
| 2 | 7532-N// | 5// | 1, 3, 5, 7, 9, 11, 13, 15, 17// | p.o.// | 32.7 | + | 8.6 | 21.5 | 339.2 | PD |
| | 121-H | 30 | 1, 3, 5, 7, 9, 11, 13, 15, 17 | p.o. | (7) | | | | (24) | |

Tumor Model GXA 3011- Exp. V1300

| | | | | | | | | | | |
|---|---|---|---|---|---|---|---|---|---|---|
| 1 | Vehicle 1// | 5 ml/kg// | 1, 3, 5, 7, 9, 11, 13, 15, 17, 19, 21// | p.o.// | n/a | n/a | 3.2 | 7.2 | 2402.5 | PD |
| | Vehicle 2 | 5 ml/kg | 1, 3, 5, 7, 9, 11, 13, 15, 17, 19, 21 | p.o. | | | | | (24) | |
| 2 | 7532-N// | 5// | 1, 3, 5, 7, 9, 11, 13, 15, 17, 19, 21// | p.o.// | 34.5 | + | 7.7 | 9.7 | 1292.9 | PD |
| | 121-H | 30 | 1, 3, 5, 7, 9, 11, 13, 15, 17, 19, 21 | p.o. | (7) | | | | (24) | |

Tumor Model GXA 3012- Exp. V1312

| | | | | | | | | | | |
|---|---|---|---|---|---|---|---|---|---|---|
| 1 | Vehicle 1// | 5 ml/kg// | 1, 3, 5, 7, 9, 11, 13, 15, 17, 19, 21// | p.o.// | n/a | n/a | 22.3 | n.r. | 158.3 | PD |
| | Vehicle 2 | 5 ml/kg | 1, 3, 5, 7, 9, 11, 13, 15, 17, 19, 21 | p.o. | | | | | (25) | |
| 2 | 7532-N// | 5// | 1, 3, 5, 7, 9, 11, 13, 15, 17, 19, 21// | p.o.// | 83.8 | − | 20.0 | n.r. | 236.6 | PD |
| | 121-H | 30 | 1, 3, 5, 7, 9, 11, 13, 15, 17, 19, 21 | p.o. | (14) | | | | (25) | |

n/a = not applicable;
n.r. = not reached (i.e. group RTVs always <200%/400%)
¹ Vehicle for 7532-N: 1% DMSO, 39.6% PEG300, 4.95% Tween 80 in PBS; vehicle for 121-H: 6% DMSO, 37.6% PEG300, 4.7% Tween 80 in PBS
²Minimum T/C values were calculated from RTV values.
Efficacy rating: ++++: T/C < 5%; +++: 5% ≤ T/C < 10%; ++: 10% ≤ T/C < 25%; +: 25% ≤ T/C < 50%; +/−: 50% ≤ T/C ≤ 65%; −: T/C > 65%
³Response R evaluation graded according to RECIST criteria: complete response (CR; R = −100%); partial response (PR; −100% < R < −50%); stable
disease (SD; −50% ≤ R < +35%); progressive disease (PD; R > 35%).
In exp. V1315, dosing was omitted in Group 2 on Days 11-21 resulting in an abridged dosing schedule.
In exp. V1316, dosing was omitted in Group 1 on Days 11-13 and Day 17; dosing was omitted in Group 2 on Days 15-17 resulting in an abridged
dosing schedule.

| | | | | | | | | | | |
|---|---|---|---|---|---|---|---|---|---|---|
| | | Dose | | | Minimum | | | | | |
| Group | | Level [mg/ | | | T/C [%] | Efficacy | Td | Tq | R [%]³ | |
| ID | Treatment ¹ | kg/day] | Schedule [Day] | Route | (Day)² | Rating | [Days] | [Days] | (Day) | RECIST³ |

Tumor Model GXA 3023, Exp. V1301

| | | | | | | | | | | |
|---|---|---|---|---|---|---|---|---|---|---|
| 1 | Vehicle 1// | 5 ml/kg// | 1, 3, 5, 7, 9, 11, 13, 15, 17, 19, 21// | p.o.// | n/a | n/a | 3.7 | 10.0 | 1980.7 | PD |
| | Vehicle 2 | 5 ml/kg | 1, 3, 5, 7, 9, 11, 13, 15, 17, 19, 21 | p.o. | | | | | (24) | |
| 2 | 7532-N// | 5// | 1, 3, 5, 7, 9, 11, 13, 15, 17, 19, 21// | p.o.// | 35.5 | + | 11.1 | 13.9 | 1356.2 | PD |
| | 121-H | 30 | 1, 3, 5, 7, 9, 11, 13, 15, 17, 19, 21 | p.o. | (10) | | | | (24) | |

Tumor Model GXA 3027, Exp. V1298

| | | | | | | | | | | |
|---|---|---|---|---|---|---|---|---|---|---|
| 1 | Vehicle 1// | 5 ml/kg// | 1, 3, 5, 7, 9, 11, 13, 15, 17, 19, 21// | p.o.// | n/a | n/a | 5.4 | 9.8 | 1448.2 | PD |
| | Vehicle 2 | 5 ml/kg | 1, 3, 5, 7, 9, 11, 13, 15, 17, 19, 21 | p.o. | | | | | (31) | |
| 2 | 7532-N// | 5// | 1, 3, 5, 7, 9, 11, 13, 15, 17, 19, 21// | p.o.// | 41.4 | + | 8.6 | 15.3 | 848.1 | PD |
| | 121-H | 30 | 1, 3, 5, 7, 9, 11, 13, 15, 17, 19, 21 | p.o. | (7) | | | | (31) | |

Tumor Model GXA 3037, Exp. V1302

| | | | | | | | | | | |
|---|---|---|---|---|---|---|---|---|---|---|
| 1 | Vehicle 1// | 5 ml/kg// | 1, 3, 5, 7, 9, 11, 13, 15, 17, 19, 21// | p.o.// | n/a | n/a | 7.9 | n.r. | 295.5 | PD |
| | Vehicle 2 | 5 ml/kg | 1, 3, 5, 7, 9, 11, 13, 15, 17, 19, 21 | p.o. | | | | | (23) | |
| 2 | 7532-N// | 5// | 1, 3, 5, 7, 9, 11, 13, 15, 17, 19, 21// | p.o.// | 83.8 | − | 8.8 | 17.8 | 471.9 | PD |
| | 121-H | 30 | 1, 3, 5, 7, 9, 11, 13, 15, 17, 19, 21 | p.o. | (9) | | | | (23) | |

n/a = not applicable;
n.r. = not reached (i.e. group RTVs always <200%/400%)
¹ Vehicle for 7532-N: 1% DMSO, 39.6% PEG300, 4.95% Tween 80 in PBS; vehicle for 121-H: 6% DMSO, 37.6% PEG300, 4.7% Tween 80 in PBS
²Minimum T/C values were calculated from RTV values.
³Response R evaluation graded according to RECIST criteria: complete response (CR; R = −100%); partial response (PR; −100% < R < −50%); stable
disease (SD; −50% ≤ R < +35%); progressive disease (PD; R > 35%).
Efficacy rating: ++++: T/C < 5%; +++: 5% ≤ T/C < 10%; ++: 10% ≤ T/C < 25%; +: 25% ≤ T/C < 50%; +/−: 50% ≤ T/C ≤ 65%; −: T/C > 65%
In exp. V1301, dosing was omitted in Group 2 on Days 17-19.

| | | | | | | | | | | |
|---|---|---|---|---|---|---|---|---|---|---|
| | | Dose | | | Minimum | | | | | |
| Group | | Level [mg/ | | | T/C [%] | Efficacy | Td | Tq | R [%]³ | |
| ID | Treatment ¹ | kg/day] | Schedule [Day] | Route | (Day)² | Rating | [Days] | [Days] | (Day) | RECIST³ |

Tumor Model GXA 3039, Exp. V1315

| | | | | | | | | | | |
|---|---|---|---|---|---|---|---|---|---|---|
| 1 | Vehicle 1// | 5 ml/kg// | 1, 3, 5, 7, 9, 11, 13, 15, 17, 19, 21// | p.o.// | n/a | n/a | 2.4 | 15.3 | 736.7 | PD |
| | Vehicle 2 | 5 ml/kg | 1, 3, 5, 7, 9, 11, 13, 15, 17, 19, 21 | p.o. | | | | | (24) | |
| 2 | 7532-N// | 5// | 1, 3, 5, 7, 9// | p.o.// | 85.4 | − | 3.6 | 13.1 | 827.4 | PD |
| | 121-H | 30 | 1, 3, 5, 7, 9 | p.o. | (3) | | | | (24) | |

Tumor Model GXA 3063, Exp. V1310

| | | | | | | | | | | |
|---|---|---|---|---|---|---|---|---|---|---|
| 1 | Vehicle 1// | 5 ml/kg// | 1, 3, 5, 7, 9, 11, 13, 15, 17, 19, 21// | p.o.// | n/a | n/a | 21.5 | n.r. | 165.4 | PD |
| | Vehicle 2 | 5 ml/kg | 1, 3, 5, 7, 9, 11, 13, 15, 17, 19, 21 | p.o. | | | | | (23) | |
| 2 | 7532-N// | 5// | 1, 3, 5, 7, 9, 11, 13, 15, 17, 19, 21// | p.o.// | 71.0 | − | n.r. | n.r. | 88.5 | PD |
| | 121-H | 30 | 1, 3, 5, 7, 9, 11, 13, 15, 17, 19, 21 | p.o. | (23) | | | | (23) | |

Tumor Model GXA 3087, Exp. V1316

| | | | | | | | | | | |
|---|---|---|---|---|---|---|---|---|---|---|
| 1 | Vehicle 1// | 5 ml/kg// | 1, 3, 5, 7, 9, 11, 13, 15// | p.o.// | n/a | n/a | 4.1 | 8.2 | 516.0 | PD |
| | Vehicle 2 | 5 ml/kg | 1, 3, 5, 7, 9, 11, 13, 15 | p.o. | | | | | (17) | |
| 2 | 7532-N// | 5// | 1, 3, 5, 7, 9, 11, 13// | p.o.// | 67.3 | − | 2.9 | 15.8 | 314.6 | PD |
| | 121-H | 30 | 1, 3, 5, 7, 9, 11, 13 | p.o. | (17) | | | | (17) | |

n/a = not applicable;
n.r. = not reached (i.e. group RTVs always <200%/400%)
¹ Vehicle for 7532-N: 1% DMSO, 39.6% PEG300, 4.95% Tween 80 in PBS; vehicle for 121-H: 6% DMSO, 37.6% PEG300, 4.7% Tween 80 in PBS
²Minimum T/C values were calculated from RTV values.
Efficacy rating: ++++: T/C < 5%; +++: 5% ≤ T/C < 10%; ++: 10% ≤ T/C < 25%; +: 25% ≤ T/C < 50%; +/−: 50% ≤ T/C ≤ 65%; −: T/C > 65%
³Response R evaluation graded according to RECIST criteria: complete response (CR; R = −100%); partial response (PR; −100% < R < −50%); stable
disease (SD; −50% ≤ R < +35%); progressive disease (PD; R > 35%).
In exp. V1315, dosing was omitted in Group 2 on Days 11-21 resulting in an abridged dosing schedule.
In exp. V1316, dosing was omitted in Group 1 on Days 11-13 and Day 17; dosing was omitted in Group 2 on Days 15-17 resulting in an abridged
dosing schedule.

| | | | | | | | | | | |
|---|---|---|---|---|---|---|---|---|---|---|
| | | Dose | | | Minimum | | | | | |
| Group | | Level [mg/ | | | T/C [%] | Efficacy | Td | Tq | R [%]³ | |
| ID | Treatment ¹ | kg/day] | Schedule [Day] | Route | (Day)² | Rating | [Days] | [Days] | (Day) | RECIST³ |

Tumor Model GXF 602, Exp. V1295

| | | | | | | | | | | |
|---|---|---|---|---|---|---|---|---|---|---|
| 1 | Vehicle 1// | 5 ml/kg// | 1, 3, 5, 7, 9, 11, 13// | p.o.// | n/a | n/a | 4.4 | 9.0 | 787.6 | PD |
| | Vehicle 2 | 5 ml/kg | 1, 3, 5, 7, 9, 11, 13 | p.o. | | | | | (21) | |
| 2 | 7532-N// | 5// | 1, 3, 5, 7, 9, 11, 13// | p.o.// | 32.0 | + | 4.5 | n.r. | 225.1 | PD |
| | 121-H | 30 | 1, 3, 5, 7, 9, 11, 13 | p.o. | (21) | | | | (24) | |

Tumor Model OVXF 550, Exp. V1296

| | | | | | | | | | | |
|---|---|---|---|---|---|---|---|---|---|---|
| 1 | Vehicle 1// | 5 ml/kg// | 1, 3, 5, 7, 9// | p.o.// | n/a | n/a | 8.2 | 17.7 | 592.5 | PD |
| | Vehicle 2 | 5 ml/kg | 1, 3, 5, 7, 9 | p.o. | | | | | (31) | |
| 2 | 7532-N// | 5// | 1, 3, 5, 7, 9, 11, 13// | p.o.// | 78.6 | − | 7.2 | 19.3 | 723.2 | PD |
| | 121-H | 30 | 1, 3, 5, 7, 9, 11, 13 | p.o. | (3) | | | | (31) | |

Tumor Model OVXF 899, Exp. V1297

| | | | | | | | | | | |
|---|---|---|---|---|---|---|---|---|---|---|
| 1 | Vehicle 1// | 5 ml/kg// | 1, 3, 5, 7, 9, 11, 13, 15, 17, 19, 21// | p.o.// | n/a | n/a | 5.3 | 8.7 | 1666.7 | PD |
| | Vehicle 2 | 5 ml/kg | 1, 3, 5, 7, 9, 11, 13, 15, 17, 19, 21 | p.o. | | | | | (21) | |
| 2 | 7532-N// | 5// | 1, 3, 5, 7, 9, 11, 13, 15, 17, 19, 21// | p.o.// | 18.9 | ++ | 11.5 | n.r. | 244.2 | PD |
| | 121-H | 30 | 1, 3, 5, 7, 9, 11, 13, 15, 17, 19, 21 | p.o. | (18) | | | | (21) | |

n/a = not applicable;
n.r. = not reached (i.e. group RTVs always <200%/400%)
¹ Vehicle for 7532-N: 1% DMSO, 39.6% PEG300, 4.95% Tween 80 in PBS; vehicle for 121-H: 6% DMSO, 37.6% PEG300, 4.7% Tween 80 in PBS
²Minimum T/C values were calculated from RTV values.
Efficacy rating: ++++: T/C < 5%; +++: 5% ≤ T/C < 10%; ++: 10% ≤ T/C < 25%; +: 25% ≤ T/C < 50%; +/−: 50% ≤ T/C ≤ 65%; −: T/C > 65%
³Response R evaluation graded according to RECIST criteria: complete response (CR; R = −100%); partial response (PR; −100% < R < −50%); stable
disease (SD; −50% ≤ R < +35%); progressive disease (PD; R > 35%).
In exp. V1296, dosing was omitted in Group 1 on Days 11-13 resulting in an abridged dosing schedule.

| | | | | | | | | | | |
|---|---|---|---|---|---|---|---|---|---|---|
| | | Dose | | | Minimum | | | | | |
| Group | | Level [mg/ | | | T/C [%] | Efficacy | Td | Tq | R [%]³ | |
| ID | Treatment ¹ | kg/day] | Schedule [Day] | Route | (Day)² | Rating | [Days] | [Days] | (Day) | RECIST³ |

Tumor Model OVXF 1320, Exp. V1317

| | | | | | | | | | | |
|---|---|---|---|---|---|---|---|---|---|---|
| 1 | Vehicle 1// | 5 ml/kg// | 1, 3, 5, 7, 9, 11, 13, 15, 17, 19, 21// | p.o.// | n/a | n/a | 1.7 | 9.8 | 1592.8 | PD |
| | Vehicle 2 | 5 ml/kg | 1, 3, 5, 7, 9, 11, 13, 15, 17, 19, 21 | p.o. | | | | | (23) | |
| 2 | 7532-N// | 5// | 1, 3, 5, 7, 9, 11, 13, 15, 17, 19, 21// | p.o.// | 1.1 | ++++ | n.r. | n.r. | −79.1 | PR |
| | 121-H | 30 | 1, 3, 5, 7, 9, 11, 13, 15, 17, 19, 21 | p.o. | (20) | | | | (23) | |

Tumor Model OVXF 1353, Exp. V1308

| | | | | | | | | | | |
|---|---|---|---|---|---|---|---|---|---|---|
| 1 | Vehicle 1// | 5 ml/kg// | 1, 3, 5, 7, 9, 11, 13, 15, 17, 19, 21// | p.o.// | n/a | n/a | 6.1 | 13.9 | 497.0 | PD |
| | Vehicle 2 | 5 ml/kg | 1, 3, 5, 7, 9, 11, 13, 15, 17, 19, 21 | p.o. | | | | | (21) | |
| 2 | 7532-N// | 5// | 1, 3, 5, 7, 9, 11, 13, 15, 17, 19, 21// | p.o.// | 53.4 | +/− | 8.1 | 19.5 | 399.3 | PD |
| | 121-H | 30 | 1, 3, 5, 7, 9, 11, 13, 15, 17, 19, 21 | p.o. | (14) | | | | (21) | |

Tumor Model GXA OVXF 1993, Exp. V1299

| | | | | | | | | | | |
|---|---|---|---|---|---|---|---|---|---|---|
| 1 | Vehicle 1// | 5 ml/kg// | 1, 3, 5, 7, 9, 11, 13, 15, 17, 19, 21// | p.o.// | n/a | n/a | 7.1 | 14.6 | 426.0 | PD |
| | Vehicle 2 | 5 ml/kg | 1, 3, 5, 7, 9, 11, 13, 15, 17, 19, 21 | p.o. | | | | | (21) | |
| 2 | 7532-N// | 5// | 1, 3, 5, 7, 9, 11, 13, 15, 17, 19, 21// | p.o.// | 46.7 | + | 11.4 | n.r. | 145.8 | PD |
| | 121-H | 30 | 1, 3, 5, 7, 9, 11, 13, 15, 17, 19, 21 | p.o. | (21) | | | | (21) | |

n/a = not applicable;
n.r. = not reached (i.e. group RTVs always <200%/400%)
¹ Vehicle for 7532-N: 1% DMSO, 39.6% PEG300, 4.95% Tween 80 in PBS; vehicle for 121-H: 6% DMSO, 37.6% PEG300, 4.7% Tween 80 in PBS
²Minimum T/C values were calculated from RTV values.
Efficacy rating: ++++: T/C < 5%; +++: 5% ≤ T/C < 10%; ++: 10% ≤ T/C < 25%; +: 25% ≤ T/C < 50%; +/−: 50% ≤ T/C ≤ 65%; −: T/C > 65%
³Response R evaluation graded according to RECIST criteria: complete response (CR; R = −100%); partial response (PR; −100% < R < −50%); stable
disease (SD; −50% ≤ R < +35%); progressive disease (PD; R > 35%).

| | | | | | | | | | | |
|---|---|---|---|---|---|---|---|---|---|---|
| | | Dose | | | Minimum | | | | | |
| Group | | Level [mg/ | | | T/C [%] | Efficacy | Td | Tq | R [%]³ | |
| ID | Treatment ¹ | kg/day] | Schedule [Day] | Route | (Day)² | Rating | [Days] | [Days] | (Day) | RECIST³ |

Tumor Model OVXF 2437, Exp. V1340

| | | | | | | | | | | |
|---|---|---|---|---|---|---|---|---|---|---|
| 1 | Vehicle 1// | 5 ml/kg// | 1, 3, 5, 7, 9, 11, 13, 15, 17, 19, 21// | p.o.// | n/a | n/a | 5.6 | 15.4 | 461.8 | PD |
| | Vehicle 2 | 5 ml/kg | 1, 3, 5, 7, 9, 11, 13, 15, 17, 19, 21 | p.o. | | | | | (24) | |
| 2 | 7532-N// | 5// | 1, 3, 5, 7, 9, 11, 13, 15, 17, 19, 21// | p.o.// | 4.2 | ++++ | n.r. | n.r. | −18.7 | SD |
| | 121-H | 30 | 1, 3, 5, 7, 9, 11, 13, 15, 17, 19, 21 | p.o. | (21) | | | | (24) | |

Tumor Model OVXF GYN090, Exp. V1311

| | | | | | | | | | | |
|---|---|---|---|---|---|---|---|---|---|---|
| 1 | Vehicle 1// | 5 ml/kg// | 1, 3, 5, 7, 9, 11, 13, 15, 17, 19, 21// | p.o.// | n/a | n/a | 6.6 | 21.4 | 526.1 | PD |
| | Vehicle 2 | 5 ml/kg | 1, 3, 5, 7, 9, 11, 13, 15, 17, 19, 21 | p.o. | | | | | (25) | |
| 2 | 7532-N// | 5// | 1, 3, 5, 7, 9, 11, 13, 15, 17, 19, 21// | p.o.// | 20.3 | ++ | 16.0 | n.r. | 26.8 | SD |
| | 121-H | 30 | 1, 3, 5, 7, 9, 11, 13, 15, 17, 19, 21 | p.o. | (25) | | | | (25) | |

Tumor Model PAXF 736, Exp. V1307

| | | | | | | | | | | |
|---|---|---|---|---|---|---|---|---|---|---|
| 1 | Vehicle 1// | 5 ml/kg// | 1, 3, 5, 7, 9, 11, 13, 15, 17, 19, 21// | p.o.// | n/a | n/a | 4.4 | 7.0 | 1172.6 | PD |
| | Vehicle 2 | 5 ml/kg | 1, 3, 5, 7, 9, 11, 13, 15, 17, 19, 21 | p.o. | | | | | (28) | |
| 2 | 7532-N/ | 5// | 1, 3, 5, 7, 9, 11, 13, 15, 17, 19, 21// | p.o.// | 75.3 | − | 5.2 | 8.8 | 1674.5 | PD |
| | 121-H | 30 | 1, 3, 5, 7, 9, 11, 13, 15, 17, 19, 21 | p.o. | (4) | | | | (28) | |

n/a = not applicable;
n.r. = not reached (i.e. group RTVs always <200%/400%)
¹ Vehicle for 7532-N: 1% DMSO, 39.6% PEG300, 4.95% Tween 80 in PBS; vehicle for 121-H: 6% DMSO, 37.6% PEG300, 4.7% Tween 80 in PBS
²Minimum T/C values were calculated from RTV values.
Efficacy rating: ++++: T/C < 5%; +++: 5% ≤ T/C < 10%; ++: 10% ≤ T/C < 25%; +: 25% ≤ T/C < 50%; +/−: 50% ≤ T/C ≤ 65%; −: T/C > 65%
³Response R evaluation graded according to RECIST criteria: complete response (CR; R = −100%); partial response (PR; −100% < R < −50%); stable
disease (SD; −50% ≤ R < +35%); progressive disease (PD; R > 35%).

| | | | | | | | | | | |
|---|---|---|---|---|---|---|---|---|---|---|
| | | Dose | | | Minimum | | | | | |
| Group | | Level [mg/ | | | T/C [%] | Efficacy | Td | Tq | R [%]³ | |
| ID | Treatment ¹ | kg/day] | Schedule [Day] | Route | (Day)² | Rating | [Days] | [Days] | (Day) | RECIST³ |

Tumor Model PAXF 1657, Exp. V1303

| | | | | | | | | | | |
|---|---|---|---|---|---|---|---|---|---|---|
| 1 | Vehicle 1// | 5 ml/kg// | 1, 3, 5, 7, 9, 11, 13, 15, 17, 19, 21// | p.o.// | n/a | n/a | 11.8 | 14.6 | 1484.9 | PD |
| | Vehicle 2 | 5 ml/kg | 1, 3, 5, 7, 9, 11, 13, 15, 17, 19, 21 | p.o. | | | | | (25) | |
| 2 | 7532-N// | 5// | 1, 3, 5, 7, 9, 11, 13, 15, 17, 19, 21// | p.o.// | 100.0 | − | 9.9 | 12.5 | 1643.5 | PD |
| | 121-H | 30 | 1, 3, 5, 7, 9, 11, 13, 15, 17, 19, 21 | p.o. | (0) | | | | (25) | |

Tumor Model PAXF 2005, Exp. V1318

| | | | | | | | | | | |
|---|---|---|---|---|---|---|---|---|---|---|
| 1 | Vehicle 1// | 5 ml/kg// | 1, 3, 5, 7, 9, 11, 13, 15, 17, 19, 21// | p.o.// | n/a | n/a | 4.0 | 6.3 | 1084.4 | PD |
| | Vehicle 2 | 5 ml/kg | 1, 3, 5, 7, 9, 11, 13, 15, 17, 19, 21 | p.o. | | | | | (25) | |
| 2 | 7532-N// | 5// | 1, 3, 5, 7, 9// | p.o.// | 34.8 | + | 6.2 | 16.3 | 1101.5 | PD |
| | 121-H | 30 | 1, 3, 5, 7, 9 | p.o. | (14) | | | | (25) | |

Tumor Model PAXF 2035, Exp. V1319

| | | | | | | | | | | |
|---|---|---|---|---|---|---|---|---|---|---|
| 1 | Vehicle 1// | 5 ml/kg// | 1, 3, 5, 7, 9, 11, 13, 15, 17, 19, 21// | p.o.// | n/a | n/a | 7.2 | 13.1 | 905.8 | PD |
| | Vehicle 2 | 5 ml/kg | 1, 3, 5, 7, 9, 11, 13, 15, 17, 19, 21 | p.o. | | | | | (25) | |
| 2 | 7532-N// | 5// | 1, 3, 5, 7, 9, 11, 13, 15, 17, 19, 21// | p.o.// | 60.7 | +/− | 11.1 | 15.9 | 797.5 | PD |
| | 121-H | 30 | 1, 3, 5, 7, 9, 11, 13, 15, 17, 19, 21 | p.o. | (14) | | | | (25) | |

n/a = not applicable;
n.r. = not reached (i.e. group RTVs always <200%/400%)
¹ Vehicle for 7532-N: 1% DMSO, 39.6% PEG300, 4.95% Tween 80 in PBS; vehicle for 121-H: 6% DMSO, 37.6% PEG300, 4.7% Tween 80 in PBS
²Minimum T/C values were calculated from RTV values.
Efficacy rating: ++++: T/C < 5%; +++: 5% ≤ T/C < 10%; ++: 10% ≤ T/C < 25%; +: 25% ≤ T/C < 50%; +/−: 50% ≤ T/C ≤ 65%; −: T/C > 65%
³Response R evaluation graded according to RECIST criteria: complete response (CR; R = −100%); partial response (PR; −100% < R < −50%); stable
disease (SD; −50% ≤ R < +35%); progressive disease (PD; R > 35%).
In exp. V1318, dosing was omitted in Group 2 on Days 11-21 resulting in an abridged dosing schedule.

| | | | | | | | | | | |
|---|---|---|---|---|---|---|---|---|---|---|
| | | Dose | | | Minimum | | | | | |
| Group | | Level [mg/ | | | T/C [%] | Efficacy | Td | Tq | R [%]³ | |
| ID | Treatment ¹ | kg/day] | Schedule [Day] | Route | (Day)² | Rating | [Days] | [Days] | (Day) | RECIST³ |

Tumor Model PAXF 2059, Exp. V1320

| | | | | | | | | | | |
|---|---|---|---|---|---|---|---|---|---|---|
| 1 | Vehicle 1// | 5 ml/kg// | 1, 3, 5, 7, 9, 11, 13, 15, 17, 19, 21// | p.o.// | n/a | n/a | 7.2 | 14.9 | 502.8 | PD |
| | Vehicle 2 | 5 ml/kg | 1, 3, 5, 7, 9, 11, 13, 15, 17, 19, 21 | p.o. | | | | | (25) | |
| 2 | 7532-N// | 5// | 1, 3, 5, 7, 9, 11, 13, 15, 17, 19, 21// | p.o.// | 88.0 | − | 4.7 | 15.3 | 658.5 | PD |
| | 121-H | 30 | 1, 3, 5, 7, 9, 11, 13, 15, 17, 19, 21 | p.o. | (14) | | | | (25) | |

Tumor Model PAXF 2082, Exp. V1194

| | | | | | | | | | | |
|---|---|---|---|---|---|---|---|---|---|---|
| 1 | Vehicle 1// | 5 ml/kg// | 1, 3, 5, 7, 9, 11, 13, 15, 17, 19, 21// | p.o.// | n/a | n/a | 6.3 | 13.4 | 1720.4 | PD |
| | Vehicle 2 | 5 ml/kg | 1, 3, 5, 7, 9, 11, 13, 15, 17, 19, 21 | p.o. | | | | | (24) | |
| 2 | 7532-N// | 5// | 1, 3, 5, 7, 9, 11, 13, 15, 17, 19, 21// | p.o.// | 65.5 | − | 5.2 | 10.0 | 1092.5 | PD |
| | 121-H | 30 | 1, 3, 5, 7, 9, 11, 13, 15, 17, 19, 21 | p.o. | (24) | | | | (24) | |

Tumor Model PAXF 2094, Exp. V1304

| | | | | | | | | | | |
|---|---|---|---|---|---|---|---|---|---|---|
| 1 | Vehicle 1// | 5 ml/kg// | 1, 3, 5, 7, 9, 11, 13, 15, 17, 19, 21// | p.o.// | n/a | n/a | 5.3 | 14.6 | 626.5 | PD |
| | Vehicle 2 | 5 ml/kg | 1, 3, 5, 7, 9, 11, 13, 15, 17, 19, 21 | p.o. | | | | | (25) | |
| 2 | 7532-N// | 5// | 1, 3, 5, 7, 9, 11, 13, 15, 17, 19, 21// | p.o.// | 54.5 | +/− | 8.2 | 15.5 | 465.6 | PD |
| | 121-H | 30 | 1, 3, 5, 7, 9, 11, 13, 15, 17, 19, 21 | p.o. | (7) | | | | (25) | |

n/a = not applicable;
n.r. = not reached (i.e. group RTVs always <200%/400%)
¹ Vehicle for 7532-N: 1% DMSO, 39.6% PEG300, 4.95% Tween 80 in PBS; vehicle for 121-H: 6% DMSO, 37.6% PEG300, 4.7% Tween 80 in PBS
²Minimum T/C values were calculated from RTV values.
Efficacy rating: ++++: T/C < 5%; +++: 5% ≤ T/C < 10%; ++: 10% ≤ T/C < 25%; +: 25% ≤ T/C < 50%; +/−: 50% ≤ T/C ≤ 65%; −: T/C > 65%
³Response R evaluation graded according to RECIST criteria: complete response (CR; R = −100%); partial response (PR; −100% < R < −50%); stable
disease (SD; −50% ≤ R < +35%); progressive disease (PD; R > 35%).

| | | | | | | | | | | |
|---|---|---|---|---|---|---|---|---|---|---|
| | | Dose | | | Minimum | | | | | |
| Group | | Level [mg/ | | | T/C [%] | Efficacy | Td | Tq | R [%]³ | |
| ID | Treatment ¹ | kg/day] | Schedule [Day] | Route | (Day)² | Rating | [Days] | [Days] | (Day) | RECIST³ |

Tumor Model PAXF 2116, Exp. V1305

| | | | | | | | | | | |
|---|---|---|---|---|---|---|---|---|---|---|
| 1 | Vehicle 1// | 5 ml/kg// | 1, 3, 5, 7, 9, 11, 13, 15, 17, 19, 21// | p.o.// | n/a | n/a | 8.2 | 14.4 | 951.8 | PD |
| | Vehicle 2 | 5 ml/kg | 1, 3, 5, 7, 9, 11, 13, 15, 17, 19, 21 | p.o. | | | | | (28) | |
| 2 | 7532-N// | 5// | 1, 3, 5, 7, 9, 11, 13, 15, 17, 19, 21// | p.o.// | 60.2 | +/− | 6.8 | 18.2 | 918.6 | PD |
| | 121-H | 30 | 1, 3, 5, 7, 9, 11, 13, 15, 17, 19, 21 | p.o. | (25) | | | | (28) | |

Tumor Model PAXF 2146, Exp. V1384

| | | | | | | | | | | |
|---|---|---|---|---|---|---|---|---|---|---|
| 1 | Vehicle 1// | 5 ml/kg// | 1, 3, 5, 7, 9, 11, 13, 15, 17, 19, 21// | p.o.// | n/a | n/a | 2.3 | 8.0 | 2444.2 | PD |
| | Vehicle 2 | 5 ml/kg | 1, 3, 5, 7, 9, 11, 13, 15, 17, 19, 21 | p.o. | | | | | (24) | |
| 2 | 7532-N// | 5// | 1, 3, 5, 7, 9, 11, 13, 15, 17, 19, 21// | p.o.// | 55.3 | +/− | 4.8 | 9.8 | 1846.9 | PD |
| | 121-H | 30 | 1, 3, 5, 7, 9, 11, 13, 15, 17, 19, 21 | p.o. | (21) | | | | (24) | |

Tumor Model PAXF 2196, Exp. V1306

| | | | | | | | | | | |
|---|---|---|---|---|---|---|---|---|---|---|
| 1 | Vehicle 1// | 5 ml/kg// | 1, 3, 5, 7, 9, 11, 13, 15, 17// | p.o.// | n/a | n/a | 4.5 | 7.3 | 2156.0 | PD |
| | Vehicle 2 | 5 ml/kg | 1, 3, 5, 7, 9, 11, 13, 15, 17 | p.o. | | | | | (18) | |
| 2 | 7532-N// | 5// | 1, 3, 5, 7, 9, 11, 13, 15, 17// | p.o.// | 23.4 | ++ | 8.5 | 12.7 | 871.0 | PD |
| | 121-H | 30 | 1, 3, 5, 7, 9, 11, 13, 15, 17 | p.o. | (11) | | | | (21) | |

n/a = not applicable;
n.r. = not reached (i.e. group RTVs always <200%/400%)
¹ Vehicle for 7532-N: 1% DMSO, 39.6% PEG300, 4.95% Tween 80 in PBS; vehicle for 121-H: 6% DMSO, 37.6% PEG300, 4.7% Tween 80 in PBS
²Minimum T/C values were calculated from RTV values.
Efficacy rating: ++++: T/C < 5%; +++: 5% ≤ T/C < 10%; ++: 10% ≤ T/C < 25%; +: 25% ≤ T/C < 50%; +/−: 50% ≤ T/C ≤ 65%; −: T/C > 65%
³Response R evaluation graded according to RECIST criteria: complete response (CR; R = −100%); partial response (PR; −100% < R < −50%); stable disease (SD; −50% ≤ R < +35%); progressive disease (PD; R > 35%).
In exp. V1306, dosing was omitted in Group 1 on Days 7-13 dosing was omitted in Group 2 on Days 19-21 resulting in an abridged dosing schedule.

TABLE 7
Body Weight Loss and Survival Rates in Each Tumor Model

| | | | | | | | | | |
|---|---|---|---|---|---|---|---|---|---|
| | | | | | | | Euthanasia | | |
| | | | | | | | for Tumor- | | Other |
| | | Dose | | Last | Maximum | Overall | Related | Adjusted | Deaths/ |
| Group | | Level [mg/ | | Day of | BWL [%] | Survival | Reasons | Survival | Euthanasia |
| ID | Treatment¹ | kg/day] | Schedule [Day] | Group | (Day)² | Rate³ | (Day) | Rate⁴ | (Day) |

Tumor Model GXA 3002- Exp. V1314

| | | | | | | | | | |
|---|---|---|---|---|---|---|---|---|---|
| 1 | Vehicle 1// | 5 ml/kg// | 1, 3, 5, 7, 9, 11// | 22 | 25.8 | 0/1 | — | 0/1 | 1 × ongoing |
| | Vehicle 2 | 5 ml/kg | 1, 3, 5, 7, 9, 11 | | (22) | (0%) | | (0%) | BWL >2 days |
| | | | | | | | | | (<80% BW) |
| | | | | | | | | | (22) |
| 2 | 7532-N// | 5// | 1, 3, 5, 7, 9, 11, 13, 15, 17// | 25 | 16.5 | 1/1 | — | 1/1 | — |
| | 121-H | 30 | 1, 3, 5, 7, 9, 11, 13, 15, 17 | | (18) | (100%) | | (100%) | |

Tumor Model GXA 3011- Exp. V1300

| | | | | | | | | | |
|---|---|---|---|---|---|---|---|---|---|
| 1 | Vehicle 1// | 5 ml/kg// | 1, 3, 5, 7, 9, 11, 13, 15, 17, 19, 21// | 25 | 7.1 | 1/1 | — | 1/1 | — |
| | Vehicle 2 | 5 ml/kg | 1, 3, 5, 7, 9, 11, 13, 15, 17, 19, 21 | | (17) | (100%) | | (100%) | |
| 2 | 7532-N// | 5// | 1, 3, 5, 7, 9, 11, 13, 15, 17, 19, 21// | 25 | 8.1 | 1/1 | — | 1/1 | — |
| | 121-H | 30 | 1, 3, 5, 7, 9, 11, 13, 15, 17, 19, 21 | | (21) | (100%) | | (100%) | |

Tumor Model GXA 3012- Exp. V1312

| | | | | | | | | | |
|---|---|---|---|---|---|---|---|---|---|
| 1 | Vehicle 1// | 5 ml/kg// | 1, 3, 5, 7, 9, 11, 13, 15, 17, 19, 21// | 25 | 5.3 | 1/1 | — | 1/1 | — |
| | Vehicle 2 | 5 ml/kg | 1, 3, 5, 7, 9, 11, 13, 15, 17, 19, 21 | | (14) | (100%) | | (100%) | |
| 2 | 7532-N// | 5// | 1, 3, 5, 7, 9, 11, 13, 15, 17, 19, 21// | 25 | 20.5 | 1/1 | — | 1/1 | — |
| | 121-H | 30 | 1, 3, 5, 7, 9, 11, 13, 15, 17, 19, 21 | | (5) | (100%) | | (100%) | |

¹Vehicle for 7532-N: 1% DMSO, 39.6% PEG300, 4.95% Tween 80 in PBS; vehicle for 121-H: 6% DMSO, 37.6% PEG300, 4.7% Tween 80 in PBS
²Day on which the minimum body weight was recorded when at least 50% remained in the group.
³Number of animals that would have survived beyond the last experimental day over total number of animals in the group.
⁴Survival rate adjusted for (i.e. including) all animals that were euthanized for tumor-related reasons.
In exp. V1314, dosing was omitted in Group 1 on Days 13-21; dosing was omitted in Group 2 on Days 19-21 resulting in an abridged dosing schedule.
In exp. V1312, dosing was omitted in Group 2 on Days 5-11.

| | | | | | | | | | |
|---|---|---|---|---|---|---|---|---|---|
| | | | | | | | Euthanasia | | |
| | | | | | | | for Tumor- | | Other |
| | | Dose | | Last | Maximum | Overall | Related | Adjusted | Deaths/ |
| Group | | Level [mg/ | | Day of | BWL [%] | Survival | Reasons | Survival | Euthanasia |
| ID | Treatment¹ | kg/day] | Schedule [Day] | Group | (Day)² | Rate³ | (Day) | Rate⁴ | (Day) |

Tumor Model GXA 3039, Exp. V1315

| | | | | | | | | | |
|---|---|---|---|---|---|---|---|---|---|
| 1 | Vehicle 1// | 5 ml/kg// | 1, 3, 5, 7, 9, 11, 13, 15, 17, 19, 21// | 25 | 16.5 | 1/1 | — | 1/1 | — |
| | Vehicle 2 | 5 ml/kg | 1, 3, 5, 7, 9, 11, 13, 15, 17, 19, 21 | | (25) | (100%) | | (100%) | |
| 2 | 7532-N// | 5// | 1, 3, 5, 7, 9// | 25 | 20.7 | 1/1 | — | 1/1 | — |
| | 121-H | 30 | 1, 3, 5, 7, 9 | | (18) | (100%) | | (100%) | |

Tumor Model GXA 3063, Exp. V1310

| | | | | | | | | | |
|---|---|---|---|---|---|---|---|---|---|
| 1 | Vehicle 1// | 5 ml/kg// | 1, 3, 5, 7, 9, 11, 13, 15, 17, 19, 21// | 24 | 6.6 | 1/1 | — | 1/1 | — |
| | Vehicle 2 | 5 ml/kg | 1, 3, 5, 7, 9, 11, 13, 15, 17, 19, 21 | | (13) | (100%) | | (100%) | |
| 2 | 7532-N// | 5// | 1, 3, 5, 7, 9, 11, 13, 15, 17, 19, 21// | 24 | 6.8 | 1/1 | — | 1/1 | — |
| | 121-H | 30 | 1, 3, 5, 7, 9, 11, 13, 15, 17, 19, 21 | | (6) | (100%) | | (100%) | |

Tumor Model GXA 3087, Exp. V1316

| | | | | | | | | | |
|---|---|---|---|---|---|---|---|---|---|
| 1 | Vehicle 1// | 5 ml/kg// | 1, 3, 5, 7, 9, 11, 13, 15// | 18 | 18.9 | 1/1 | — | 1/1 | — |
| | Vehicle 2 | 5 ml/kg | 1, 3, 5, 7, 9, 11, 13, 15 | | (18) | (100%) | | (100%) | |
| 2 | 7532-N// | 5// | 1, 3, 5, 7, 9, 11, 13// | 18 | 27.9 | 0/1 | — | 0/1 | 1 × ongoing |
| | 121-H | 30 | 1, 3, 5, 7, 9, 11, 13 | | (18) | (0%) | | (0%) | BWL >2 days |
| | | | | | | | | | (<80% BW) |
| | | | | | | | | | (18) |

¹Vehicle for 7532-N: 1% DMSO, 39.6% PEG300, 4.95% Tween 80 in PBS; vehicle for 121-H: 6% DMSO, 37.6% PEG300, 4.7% Tween 80 in PBS
²Day on which the minimum body weight was recorded when at least 50% remained in the group; n.r.: not relevant, no body weight loss recorded (i.e.
group RBW always >100%).
³Number of animals that would have survived beyond the last experimental day over total number of animals in the group.
⁴Survival rate adjusted for (i.e. including) all animals that were euthanized for tumor-related reasons.
In exp. V1301, dosing was omitted in Group 2 on Days 17-19.

| | | | | | | | | | |
|---|---|---|---|---|---|---|---|---|---|
| | | | | | | | Euthanasia | | |
| | | | | | | | for Tumor- | | Other |
| | | Dose | | Last | Maximum | Overall | Related | Adjusted | Deaths/ |
| Group | | Level [mg/ | | Day of | BWL [%] | Survival | Reasons | Survival | Euthanasia |
| ID | Treatment¹ | kg/day] | Schedule [Day] | Group | (Day)² | Rate³ | (Day) | Rate⁴ | (Day) |

Tumor Model GXA 3039, Exp. V1315

| | | | | | | | | | |
|---|---|---|---|---|---|---|---|---|---|
| 1 | Vehicle 1// | 5 ml/kg// | 1, 3, 5, 7, 9, 11, 13, 15, 17, 19, 21// | 25 | 16.5 | 1/1 | — | 1/1 | — |
| | Vehicle 2 | 5 ml/kg | 1, 3, 5, 7, 9, 11, 13, 15, 17, 19, 21 | | (25) | (100%) | | (100%) | |
| 2 | 7532-N// | 5// | 1, 3, 5, 7, 9// | 25 | 20.7 | 1/1 | — | 1/1 | — |
| | 121-H | 30 | 1, 3, 5, 7, 9 | | (18) | (100%) | | (100%) | |

Tumor Model GXA 3063, Exp. V1310

| | | | | | | | | | |
|---|---|---|---|---|---|---|---|---|---|
| 1 | Vehicle 1// | 5 ml/kg// | 1, 3, 5, 7, 9, 11, 13, 15, 17, 19, 21// | 24 | 6.6 | 1/1 | — | 1/1 | — |
| | Vehicle 2 | 5 ml/kg | 1, 3, 5, 7, 9, 11, 13, 15, 17, 19, 21 | | (13) | (100%) | | (100%) | |
| 2 | 7532-N// | 5// | 1, 3, 5, 7, 9, 11, 13, 15, 17, 19, 21// | 24 | 6.8 | 1/1 | — | 1/1 | — |
| | 121-H | 30 | 1, 3, 5, 7, 9, 11, 13, 15, 17, 19, 21 | | (6) | (100%) | | (100%) | |

Tumor Model GXA 3087, Exp. V1316

| | | | | | | | | | |
|---|---|---|---|---|---|---|---|---|---|
| 1 | Vehicle 1// | 5 ml/kg// | 1, 3, 5, 7, 9, 11, 13, 15// | 18 | 18.9 | 1/1 | — | 1/1 | — |
| | Vehicle 2 | 5 ml/kg | 1, 3, 5, 7, 9, 11, 13, 15 | | (18) | (100%) | | (100%) | |
| 2 | 7532-N// | 5// | 1, 3, 5, 7, 9, 11, 13// | 18 | 27.9 | 0/1 | — | 0/1 | 1 × ongoing |
| | 121-H | 30 | 1, 3, 5, 7, 9, 11, 13 | | (18) | (0%) | | (0%) | BWL >2 days |
| | | | | | | | | | (<80% BW) |
| | | | | | | | | | (18) |

¹Vehicle for 7532-N: 1% DMSO, 39.6% PEG300, 4.95% Tween 80 in PBS; vehicle for 121-H: 6% DMSO, 37.6% PEG300, 4.7% Tween 80 in PBS
²Day on which the minimum body weight was recorded when at least 50% remained in the group; n.r.: not relevant, no body weight loss recorded (i.e.
group RBW always >100%).
³Number of animals that would have survived beyond the last experimental day over total number of animals in the group.
⁴Survival rate adjusted for (i.e. including) all animals that were euthanized for tumor-related reasons.
In exp. V1315, dosing was omitted in Group 2 on Days 11-21 resulting in an abridged dosing schedule.
In exp. V1316, dosing was omitted in Group 1 on Days 11-13 and Day 17; dosing was omitted in Group 2 on Days 15-17 resulting in an
abridged dosing schedule.

| | | | | | | | | | |
|---|---|---|---|---|---|---|---|---|---|
| | | | | | | | Euthanasia | | |
| | | | | | | | for Tumor- | | Other |
| | | Dose | | Last | Maximum | Overall | Related | Adjusted | Deaths/ |
| Group | | Level [mg/ | | Day of | BWL [%] | Survival | Reasons | Survival | Euthanasia |
| ID | Treatment¹ | kg/day] | Schedule [Day] | Group | (Day)² | Rate³ | (Day) | Rate⁴ | (Day) |

Tumor Model GXA 3039, Exp. V1315

| | | | | | | | | | |
|---|---|---|---|---|---|---|---|---|---|
| 1 | Vehicle 1// | 5 ml/kg// | 1, 3, 5, 7, 9, 11, 13, 15, 17, 19, 21// | 25 | 16.5 | 1/1 | — | 1/1 | — |
| | Vehicle 2 | 5 ml/kg | 1, 3, 5, 7, 9, 11, 13, 15, 17, 19, 21 | | (25) | (100%) | | (100%) | |
| 2 | 7532-N// | 5// | 1, 3, 5, 7, 9// | 25 | 20.7 | 1/1 | — | 1/1 | — |
| | 121-H | 30 | 1, 3, 5, 7, 9 | | (18) | (100%) | | (100%) | |

Tumor Model GXA 3063, Exp. V1310

| | | | | | | | | | |
|---|---|---|---|---|---|---|---|---|---|
| 1 | Vehicle 1// | 5 ml/kg// | 1, 3, 5, 7, 9, 11, 13, 15, 17, 19, 21// | 24 | 6.6 | 1/1 | — | 1/1 | — |
| | Vehicle 2 | 5 ml/kg | 1, 3, 5, 7, 9, 11, 13, 15, 17, 19, 21 | | (13) | (100%) | | (100%) | |
| 2 | 7532-N// | 5// | 1, 3, 5, 7, 9, 11, 13, 15, 17, 19, 21// | 24 | 6.8 | 1/1 | — | 1/1 | — |
| | 121-H | 30 | 1, 3, 5, 7, 9, 11, 13, 15, 17, 19, 21 | | (6) | (100%) | | (100%) | |

Tumor Model GXA 3087, Exp. V1316

| | | | | | | | | | |
|---|---|---|---|---|---|---|---|---|---|
| 1 | Vehicle 1// | 5 ml/kg// | 1, 3, 5, 7, 9, 11, 13, 15// | 18 | 18.9 | 1/1 | — | 1/1 | — |
| | Vehicle 2 | 5 ml/kg | 1, 3, 5, 7, 9, 11, 13, 15 | | (18) | (100%) | | (100%) | |
| 2 | 7532-N// | 5// | 1, 3, 5, 7, 9, 11, 13// | 18 | 27.9 | 0/1 | — | 0/1 | 1 × ongoing |
| | 121-H | 30 | 1, 3, 5, 7, 9, 11, 13 | | (18) | (0%) | | (0%) | BWL >2 days |
| | | | | | | | | | (<80% BW) |
| | | | | | | | | | (18) |

¹Vehicle for 7532-N: 1% DMSO, 39.6% PEG300, 4.95% Tween 80 in PBS; vehicle for 121-H: 6% DMSO, 37.6% PEG300, 4.7% Tween 80 in PBS
²Day on which the minimum body weight was recorded when at least 50% remained in the group; n.r.: not relevant, no body weight loss recorded (i.e.
group RBW always >100%).
³Number of animals that would have survived beyond the last experimental day over total number of animals in the group.
⁴Survival rate adjusted for (i.e. including) all animals that were euthanized for tumor-related reasons.
In exp. V1296, dosing was omitted in Group 1 on Days 11-13 resulting in an abridged dosing schedule.

| | | | | | | | | | |
|---|---|---|---|---|---|---|---|---|---|
| | | | | | | | Euthanasia | | |
| | | | | | | | for Tumor- | | Other |
| | | Dose | | Last | Maximum | Overall | Related | Adjusted | Deaths/ |
| Group | | Level [mg/ | | Day of | BWL [%] | Survival | Reasons | Survival | Euthanasia |
| ID | Treatment¹ | kg/day] | Schedule [Day] | Group | (Day)² | Rate³ | (Day) | Rate⁴ | (Day) |

Tumor Model GXF 602, Exp. V1295

| | | | | | | | | | |
|---|---|---|---|---|---|---|---|---|---|
| 1 | Vehicle 1// | 5 ml/kg// | 1, 3, 5, 7, 9, 11, 13// | 21 | 1.4 | 0/1 | 1 × | 1/1 | — |
| | Vehicle 2 | 5 ml/kg | 1, 3, 5, 7, 9, 11, 13 | | (3) | (0%) | ATV >2000 | (100%) | |
| | | | | | | | mm³ | | |
| | | | | | | | (21) | | |
| 2 | 7532-N// | 5// | 1, 3, 5, 7, 9, 11, 13// | 25 | 5.3 | 1/1 | — | 1/1 | — |
| | 121-H | 30 | 1, 3, 5, 7, 9, 11, 13 | | (7) | (100%) | | (100%) | |

Tumor Model OVXF 550, Exp. V1296

| | | | | | | | | | |
|---|---|---|---|---|---|---|---|---|---|
| 1 | Vehicle 1// | 5 ml/kg// | 1, 3, 5, 7, 9// | 32 | 21.3 | 1/1 | — | 1/1 | — |
| | Vehicle 2 | 5 ml/kg | 1, 3, 5, 7, 9 | | (32) | (100%) | | (100%) | |
| 2 | 7532-N// | 5// | 1, 3, 5, 7, 9, 11, 13// | 32 | 5.4 | 1/1 | — | 1/1 | — |
| | 121-H | 30 | 1, 3, 5, 7, 9, 11, 13 | | (28) | (100%) | | (100%) | |

Tumor Model OVXF 899, Exp. V1297

| | | | | | | | | | |
|---|---|---|---|---|---|---|---|---|---|
| 1 | Vehicle 1// | 5 ml/kg// | 1, 3, 5, 7, 9, 11, 13, 15, 17, 19, 21// | 22 | 14.3 | 1/1 | — | 1/1 | — |
| | Vehicle 2 | 5 ml/kg | 1, 3, 5, 7, 9, 11, 13, 15, 17, 19, 21 | | (22) | (100%) | | (100%) | |
| 2 | 7532-N// | 5// | 1, 3, 5, 7, 9, 11, 13, 15, 17, 19, 21// | 22 | 7.8 | 1/1 | — | 1/1 | — |
| | 121-H | 30 | 1, 3, 5, 7, 9, 11, 13, 15, 17, 19, 21 | | (14) | (100%) | | (100%) | |

¹Vehicle for 7532-N: 1% DMSO, 39.6% PEG300, 4.95% Tween 80 in PBS; vehicle for 121-H: 6% DMSO, 37.6% PEG300, 4.7% Tween 80 in PBS
²Day on which the minimum body weight was recorded when at least 50% remained in the group; n.r.: not relevant, no body weight loss recorded (i.e.
group RBW always >100%).
³Number of animals that would have survived beyond the last experimental day over total number of animals in the group.
⁴Survival rate adjusted for (i.e. including) all animals that were euthanized for tumor-related reasons.

| | | | | | | | | | |
|---|---|---|---|---|---|---|---|---|---|
| | | | | | | | Euthanasia | | |
| | | | | | | | for Tumor- | | Other |
| | | Dose | | Last | Maximum | Overall | Related | Adjusted | Deaths/ |
| Group | | Level [mg/ | | Day of | BWL [%] | Survival | Reasons | Survival | Euthanasia |
| ID | Treatment¹ | kg/day] | Schedule [Day] | Group | (Day)² | Rate³ | (Day) | Rate⁴ | (Day) |

Tumor Model OVXF 2437, Exp. V1340

| | | | | | | | | | |
|---|---|---|---|---|---|---|---|---|---|
| 1 | Vehicle 1// | 5 ml/kg// | 1, 3, 5, 7, 9, 11, 13, 15, 17, 19, 21// | 25 | n.r. | 1/1 | — | 1/1 | — |
| | Vehicle 2 | 5 ml/kg | 1, 3, 5, 7, 9, 11, 13, 15, 17, 19, 21 | | | (100%) | | (100%) | |
| 2 | 7532-N// | 5// | 1, 3, 5, 7, 9, 11, 13, 15, 17, 19, 21// | 25 | n.r. | 1/1 | — | 1/1 | — |
| | 121-H | 30 | 1, 3, 5, 7, 9, 11, 13, 15, 17, 19, 21 | | | (100%) | | (100%) | |

Tumor Model OVXF GYN090, Exp. V1311

| | | | | | | | | | |
|---|---|---|---|---|---|---|---|---|---|
| 1 | Vehicle 1// | 5 ml/kg// | 1, 3, 5, 7, 9, 11, 13, 15, 17, 19, 21// | 25 | 11.4 | 1/1 | — | 1/1 | — |
| | Vehicle 2 | 5 ml/kg | 1, 3, 5, 7, 9, 11, 13, 15, 17, 19, 21 | | (14) | (100%) | | (100%) | |
| 2 | 7532-N// | 5// | 1, 3, 5, 7, 9, 11, 13, 15, 17, 19, 21// | 25 | 12.4 | 1/1 | — | 1/1 | — |
| | 121-H | 30 | 1, 3, 5, 7, 9, 11, 13, 15, 17, 19, 21 | | (13) | (100%) | | (100%) | |

Tumor Model PAXF 736, Exp. V1307

| | | | | | | | | | |
|---|---|---|---|---|---|---|---|---|---|
| 1 | Vehicle 1// | 5 ml/kg// | 1, 3, 5, 7, 9, 11, 13, 15, 17, 19, 21// | 29 | 2.7 | 1/1 | — | 1/1 | — |
| | Vehicle 2 | 5 ml/kg | 1, 3, 5, 7, 9, 11, 13, 15, 17, 19, 21 | | (4) | (100%) | | (100%) | |
| 2 | 7532-N// | 5// | 1, 3, 5, 7, 9, 11, 13, 15, 17, 19, 21// | 29 | 6.4 | 1/1 | — | 1/1 | — |
| | 121-H | 30 | 1, 3, 5, 7, 9, 11, 13, 15, 17, 19, 21 | | (7) | (100%) | | (100%) | |

¹Vehicle for 7532-N: 1% DMSO, 39.6% PEG300, 4.95% Tween 80 in PBS; vehicle for 121-H: 6% DMSO, 37.6% PEG300, 4.7% Tween 80 in PBS
²Day on which the minimum body weight was recorded when at least 50% remained in the group; n.r.: not relevant, no body weight loss recorded (i.e.
group RBW always >100%).
³Number of animals that would have survived beyond the last experimental day over total number of animals in the group.
⁴Survival rate adjusted for (i.e. including) all animals that were euthanized for tumor-related reasons.

| | | | | | | | | | |
|---|---|---|---|---|---|---|---|---|---|
| | | | | | | | Euthanasia | | |
| | | | | | | | for Tumor- | | Other |
| | | Dose | | Last | Maximum | Overall | Related | Adjusted | Deaths/ |
| Group | | Level [mg/ | | Day of | BWL [%] | Survival | Reasons | Survival | Euthanasia |
| ID | Treatment¹ | kg/day] | Schedule [Day] | Group | (Day)² | Rate³ | (Day) | Rate⁴ | (Day) |

Tumor Model PAXF 1657, Exp. V1303

| | | | | | | | | | |
|---|---|---|---|---|---|---|---|---|---|
| 1 | Vehicle 1// | 5 ml/kg// | 1, 3, 5, 7, 9, 11, 13, 15, 17, 19, 21// | 25 | 4.3 | 1/1 | — | 1/1 | — |
| | Vehicle 2 | 5 ml/kg | 1, 3, 5, 7, 9, 11, 13, 15, 17, 19, 21 | | (11) | (100%) | | (100%) | |
| 2 | 7532-N// | 5// | 1, 3, 5, 7, 9, 11, 13, 15, 17, 19, 21// | 25 | 8.6 | 1/1 | — | 1/1 | — |
| | 121-H | 30 | 1, 3, 5, 7, 9, 11, 13, 15, 17, 19, 21 | | (11) | (100%) | | (100%) | |

Tumor Model PAXF 2005, Exp. V1318

| | | | | | | | | | |
|---|---|---|---|---|---|---|---|---|---|
| 1 | Vehicle 1// | 5 ml/kg// | 1, 3, 5, 7, 9, 11, 13, 15, 17, 19, 21// | 25 | 10.0 | 1/1 | — | 1/1 | — |
| | Vehicle 2 | 5 ml/kg | 1, 3, 5, 7, 9, 11, 13, 15, 17, 19, 21 | | (18) | (100%) | | (100%) | |
| 2 | 7532-N// | 5// | 1, 3, 5, 7, 9// | 25 | 22.3 | 1/2 | | 1/2 | 1 × found dead |
| | 121-H | 30 | 1, 3, 5, 7, 9 | | (17) | (50%) | | (50%) | (3) |

Tumor Model PAXF 2035, Exp. V1319

| | | | | | | | | | |
|---|---|---|---|---|---|---|---|---|---|
| 1 | Vehicle 1// | 5 ml/kg// | 1, 3, 5, 7, 9, 11, 13, 15, 17, 19, 21// | 25 | 6.8 | 1/1 | — | 1/1 | — |
| | Vehicle 2 | 5 ml/kg | 1, 3, 5, 7, 9, 11, 13, 15, 17, 19, 21 | | (14) | (100%) | | (100%) | |
| 2 | 7532-N// | 5// | 1, 3, 5, 7, 9, 11, 13, 15, 17, 19, 21// | 25 | n.r. | 1/1 | — | 1/1 | — |
| | 121-H | 30 | 1, 3, 5, 7, 9, 11, 13, 15, 17, 19, 21 | | | (100%) | | (100%) | |

¹Vehicle for 7532-N: 1% DMSO, 39.6% PEG300, 4.95% Tween 80 in PBS; vehicle for 121-H: 6% DMSO, 37.6% PEG300, 4.7% Tween 80 in PBS
²Day on which the minimum body weight was recorded when at least 50% remained in the group; n.r.: not relevant, no body weight loss recorded (i.e.
group RBW always >100%).
³Number of animals that would have survived beyond the last experimental day over total number of animals in the group.
⁴Survival rate adjusted for (i.e. including) all animals that were euthanized for tumor-related reasons.
In exp. V1318, dosing was omitted in Group 2 on Days 11-21 resulting in an abridged dosing schedule.
In exp. V1318, Group 2 animal found dead on Day 3 was replaced by another animal.

| | | | | | | | | | |
|---|---|---|---|---|---|---|---|---|---|
| | | | | | | | Euthanasia | | |
| | | | | | | | for Tumor- | | Other |
| | | Dose | | Last | Maximum | Overall | Related | Adjusted | Deaths/ |
| Group | | Level [mg/ | | Day of | BWL [%] | Survival | Reasons | Survival | Euthanasia |
| ID | Treatment¹ | kg/day] | Schedule [Day] | Group | (Day)² | Rate³ | (Day) | Rate⁴ | (Day) |

Tumor Model PAXF 2059, Exp. V1320

| | | | | | | | | | |
|---|---|---|---|---|---|---|---|---|---|
| 1 | Vehicle 1// | 5 ml/kg// | 1, 3, 5, 7, 9, 11, 13, 15, 17, 19, 21// | 25 | 6.6 | 1/1 | — | 1/1 | — |
| | Vehicle 2 | 5 ml/kg | 1, 3, 5, 7, 9, 11, 13, 15, 17, 19, 21 | | (18) | (100%) | | (100%) | |
| 2 | 7532-N// | 5// | 1, 3, 5, 7, 9, 11, 13, 15, 17, 19, 21// | 25 | 5.5 | 1/1 | — | 1/1 | — |
| | 121-H | 30 | 1, 3, 5, 7, 9, 11, 13, 15, 17, 19, 21 | | (7) | (100%) | | (100%) | |

Tumor Model PAXF 2082, Exp. V1194

| | | | | | | | | | |
|---|---|---|---|---|---|---|---|---|---|
| 1 | Vehicle 1// | 5 ml/kg// | 1, 3, 5, 7, 9, 11, 13, 15, 17, 19, 21// | 25 | 7.4 | 1/1 | — | 1/1 | — |
| | Vehicle 2 | 5 ml/kg | 1, 3, 5, 7, 9, 11, 13, 15, 17, 19, 21 | | (14) | (100%) | | (100%) | |
| 2 | 7532-N// | 5// | 1, 3, 5, 7, 9, 11, 13, 15, 17, 19, 21// | 25 | 3.2 | 1/1 | — | 1/1 | — |
| | 121-H | 30 | 1, 3, 5, 7, 9, 11, 13, 15, 17, 19, 21 | | (7) | (100%) | | (100%) | |

Tumor Model PAXF 2094, Exp. V1304

| | | | | | | | | | |
|---|---|---|---|---|---|---|---|---|---|
| 1 | Vehicle 1// | 5 ml/kg// | 1, 3, 5, 7, 9, 11, 13, 15, 17, 19, 21// | 25 | n.r. | 1/1 | — | 1/1 | — |
| | Vehicle 2 | 5 ml/kg | 1, 3, 5, 7, 9, 11, 13, 15, 17, 19, 21 | | (—) | (100%) | | (100%) | |
| 2 | 7532-N// | 5// | 1, 3, 5, 7, 9, 11, 13, 15, 17, 19, 21// | 25 | 5.7 | 1/1 | — | 1/1 | — |
| | 121-H | 30 | 1, 3, 5, 7, 9, 11, 13, 15, 17, 19, 21 | | (25) | (100%) | | (100%) | |

¹Vehicle for 7532-N: 1% DMSO, 39.6% PEG300, 4.95% Tween 80 in PBS; vehicle for 121-H: 6% DMSO, 37.6% PEG300, 4.7% Tween 80 in PBS
²Day on which the minimum body weight was recorded when at least 50% remained in the group; n.r.: not relevant, no body weight loss recorded (i.e.
group RBW always >100%).
³Number of animals that would have survived beyond the last experimental day over total number of animals in the group.
⁴Survival rate adjusted for (i.e. including) all animals that were euthanized for tumor-related reasons.

| | | | | | | | | | |
|---|---|---|---|---|---|---|---|---|---|
| | | | | | | | Euthanasia | | |
| | | | | | | | for Tumor- | | Other |
| | | Dose | | Last | Maximum | Overall | Related | Adjusted | Deaths/ |
| Group | | Level [mg/ | | Day of | BWL [%] | Survival | Reasons | Survival | Euthanasia |
| ID | Treatment¹ | kg/day] | Schedule [Day] | Group | (Day)² | Rate³ | (Day) | Rate⁴ | (Day) |

Tumor Model PAXF 2116, Exp. V1305

| | | | | | | | | | |
|---|---|---|---|---|---|---|---|---|---|
| 1 | Vehicle 1// | 5 ml/kg// | 1, 3, 5, 7, 9, 11, 13, 15, 17, 19, 21// | 29 | 4.4 | 1/1 | — | 1/1 | — |
| | Vehicle 2 | 5 ml/kg | 1, 3, 5, 7, 9, 11, 13, 15, 17, 19, 21 | | (14) | (100%) | | (100%) | |
| 2 | 7532-N// | 5// | 1, 3, 5, 7, 9, 11, 13, 15, 17, 19, 21// | 29 | 5.5 | 1/1 | — | 1/1 | — |
| | 121-H | 30 | 1, 3, 5, 7, 9, 11, 13, 15, 17, 19, 21 | | (7) | (100%) | | (100%) | |

Tumor Model PAXF 2146, Exp. V1384

| | | | | | | | | | |
|---|---|---|---|---|---|---|---|---|---|
| 1 | Vehicle 1// | 5 ml/kg// | 1, 3, 5, 7, 9, 11, 13, 15, 17, 19, 21// | 25 | 2.2 | 1/1 | — | 1/1 | — |
| | Vehicle 2 | 5 ml/kg | 1, 3, 5, 7, 9, 11, 13, 15, 17, 19, 21 | | (7) | (100%) | | (100%) | |
| 2 | 7532-N// | 5// | 1, 3, 5, 7, 9, 11, 13, 15, 17, 19, 21// | 25 | n.r. | 1/1 | — | 1/1 | — |
| | 121-H | 30 | 1, 3, 5, 7, 9, 11, 13, 15, 17, 19, 21 | | (—) | (100%) | | (100%) | |

Tumor Model PAXF 2196, Exp. V1306

| | | | | | | | | | |
|---|---|---|---|---|---|---|---|---|---|
| 1 | Vehicle 1// | 5 ml/kg// | 1, 3, 5, 7, 9, 11, 13, 15, 17// | 18 | 15.4 | 0/1 | 1 × | 1/1 | — |
| | Vehicle 2 | 5 ml/kg | 1, 3, 5, 7, 9, 11, 13, 15, 17 | | (7) | (0%) | ATV >2000 | (100%) | |
| | | | | | | | mm³ | | |
| | | | | | | | (18) | | |
| 2 | 7532-N// | 5// | 1, 3, 5, 7, 9, 11, 13, 15, 17// | 22 | 17.7 | 1/1 | — | 1/1 | — |
| | 121-H | 30 | 1, 3, 5, 7, 9, 11, 13, 15, 17 | | (20) | (100%) | | (100%) | |

¹Vehicle for 7532-N: 1% DMSO, 39.6% PEG300, 4.95% Tween 80 in PBS; vehicle for 121-H: 6% DMSO, 37.6% PEG300, 4.7% Tween 80 in PBS
²Day on which the minimum body weight was recorded when at least 50% remained in the group; n.r.: not relevant, no body weight loss recorded (i.e. group RBW always >100%).
³Number of animals that would have survived beyond the last experimental day over total number of animals in the group.
⁴Survival rate adjusted for (i.e. including) all animals that were euthanized for tumor-related reasons.
Dosing was omitted in exp. V1314 Group 1 on Days 13-21; dosing was omitted in Group 2 on Days 19-21 resulting in an abridged dosing schedule.

### Example 9: Efficacy of Compositions Described Herein on Inflammatory Diseases

The present example further demonstrates treatment of diseases associated with inflammation and/or fibrosis. Specifically, the present example demonstrates lethality of compositions described herein to pathogenic stem cells isolated from Crohn's disease patients.

Approximately 2,000 pathogenic inflammatory gastric metaplasia stem cells from Crohn's terminal ileum were seeded on multiple 384-well plates (Griener Bio-One, USA). 1-day post seeding, indicated compounds were added by automation at the High Throughput Research and Screening Center of the Institute of Biosciences and Technology at the Texas A&M University (Houston, Texas). After treatment, plates were sealed with breathable membranes and maintained for 4 days in a 37° C., 7.5% CO2 incubator. Plates were then fixed, stained, and imaged. In brief, the treated 384-well plates were washed with phosphate buffered saline (Gibco, USA) and fixed with 4% paraformaldehyde at room temperature for 25 minutes. After fixation, plates were then stained with DAPI for 1 hr at room temperature before imaging via a high-content automatic screening system (Thermo Scientific CellInsight CX7 LED, Thermo Fisher Scientific, Waltham, MA, USA).

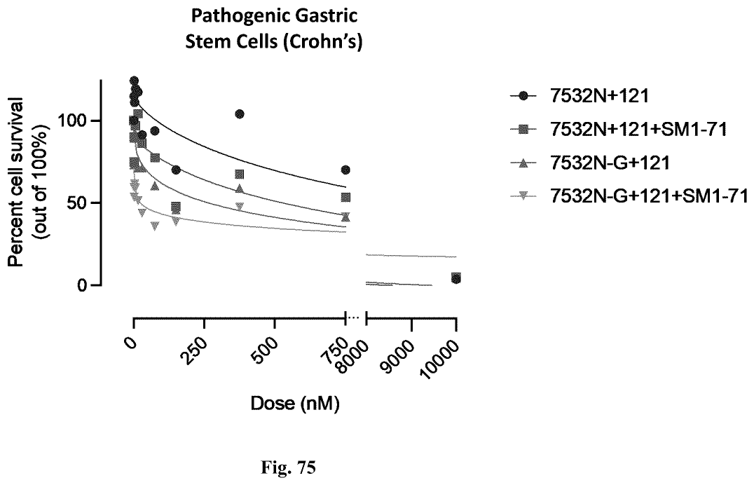

FIG. 75 shows treatment of pathogenic gastric stem cells with compositions described herein resulted in lethality at a low dose with dose response curves.

1. A pharmaceutical composition comprising an IAP inhibitor and a cellular kinase inhibitor wherein the IAP inhibitor is a compound of formula I: 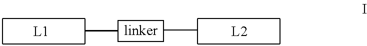
or a pharmaceutically acceptable salt thereof,
wherein:
L1 is a first ligand;
L2 is a second ligand; and
linker is a bivalent linker comprising

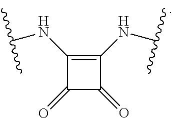 [SMILES: *Nc1c(N*)c(=O)c1=O]

2. The pharmaceutical composition of claim 1, wherein the pharmaceutical composition is a single entity.

3. The pharmaceutical composition of claim 2, wherein the pharmaceutical composition is a unit dosage form.

4. A combination comprising:
an IAP inhibitor; and
a cellular kinase inhibitor
wherein the IAP inhibitor is a compound of formula I: 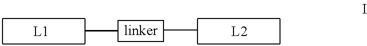
or a pharmaceutically acceptable salt thereof,
wherein:
L1 is a first ligand;
L2 is a second ligand; and
linker is a bivalent linker comprising

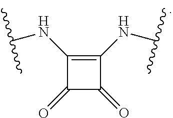 [SMILES: *Nc1c(N*)c(=O)c1=O]

5. (canceled)

6. A method of treating a proliferative disorder comprising administering to a subject in need thereof combination therapy with an IAP inhibitor and a cellular kinase inhibitor
wherein the IAP inhibitor is a compound of formula I: 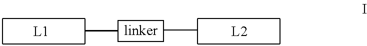
or a pharmaceutically acceptable salt thereof,
wherein:
L1 is a first ligand;
L2 is a second ligand; and
linker is a bivalent linker comprising

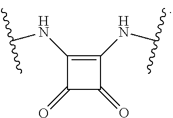 [SMILES: *Nc1c(N*)c(=O)c1=O]

7. The method of claim 6 comprising administering the IAP inhibitor to the subject in need thereof wherein the subject has received or is receiving the cellular kinase inhibitor.

8. The method of claim 6 comprising administering the cellular kinase inhibitor to he subject in need thereof wherein the subject has received or is receiving the IAP inhibitor.

9. (canceled)

10. The method of claim 6 wherein the proliferative disorder is a pulmonary disease or disorder.

11. The method of claim 6 wherein the proliferative disorder is a cancer.

12. The method of claim 6 wherein the cellular kinase inhibitor and the IAP inhibitor are delivered in the same composition.

13. The method of claim 6 wherein the cellular kinase inhibitor and the IAP inhibitor are not delivered in the same composition.

14. The method of claim 6 wherein the cellular kinase inhibitor is a tyrosine kinase inhibitor.

15. The method of claim 14 wherein the cellular kinase inhibitor is ponatinib.

16. (canceled)

17. The method of claim 6, wherein each of L1 and L2 is independently a moiety that binds to one or more Inhibitor of Apoptosis Proteins (IAPs).

18. (canceled)

19. The composition or method of claim 17, wherein each of L1 and L2 independently comprises a group selected from: 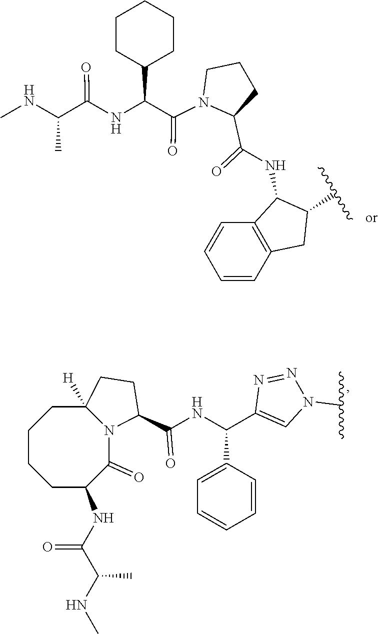 [SMILES: *[C@@H]1Cc2ccccc2[C@@H]1NC(=O)[C@@H]1CCCN1C(=O)[C@@H](NC(=O)[C@H](C)NC)C1CCCCC1 ; *n1cc([C@@H](NC(=O)[C@@H]2CC[C@@H]3CCCC[C@H](NC(=O)[C@H](C)NC)C(=O)N32)c2ccccc2)nn1]
or a pharmaceutically acceptable salt thereof.

20.-23. (canceled)

24. The composition or method of claim 17, wherein the compound is of formula I-a, I-b, or I-c: 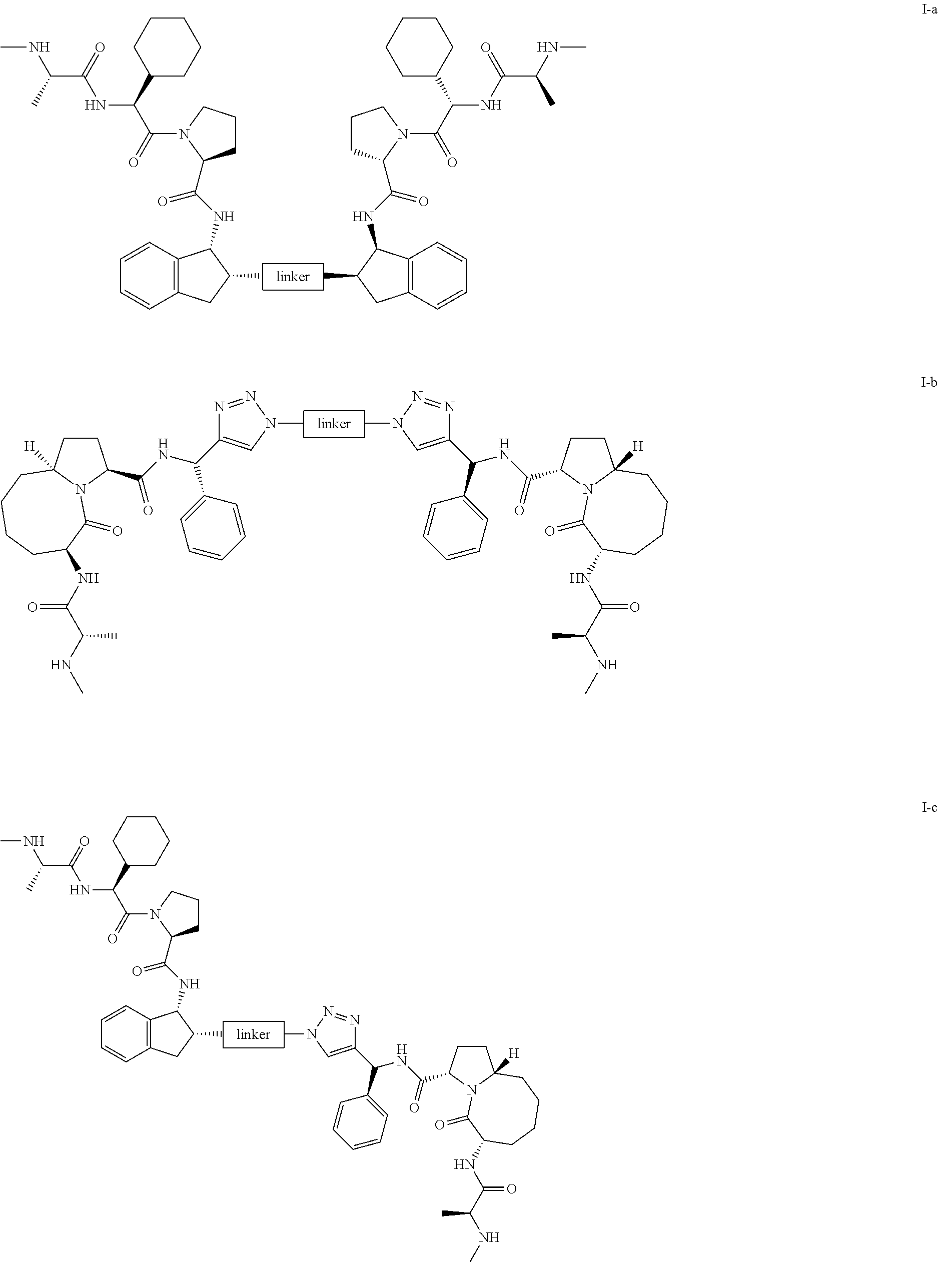
or a pharmaceutically acceptable salt thereof.

25. The method of claim 6, wherein the linker is of formula X: 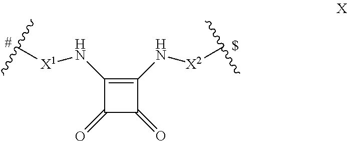
or a pharmaceutically acceptable salt thereof, wherein:
each of X¹ and X² is independently a covalent bond or an optionally substituted bivalent, saturated or partially unsaturated, straight or branched C1-12 hydrocarbon chain, wherein 1-4 carbon atoms are optionally and independently replaced by —O—, —N(R)—, —C(O)—, —S—, —SO—, —SO₂—, or -Cy-;
each R is independently selected from hydrogen or an optionally substituted C₁₋₆ aliphatic;
each -Cy- is independently an optionally substituted bivalent ring selected from a 3- to 8-membered carbocyclene, a 5- to 6-membered saturated or partially unsaturated heterocyclene having 1-3 heteroatoms independently selected from oxygen, nitrogen, or sulfur; phenylene; or a 5- to 6-membered heteroarylene having 1-3 heteroatoms independently selected from oxygen, nitrogen, or sulfur;
#represents the point of attachment to L1; and
$ represents the point of attachment to L2.

26. The method of claim 25, wherein X¹ and X² are the same.

27. The method of claim 25, wherein X¹ and X² are different.

28.-36. (canceled)

37. The method of claim 6, wherein X¹ is:
covalent bond, 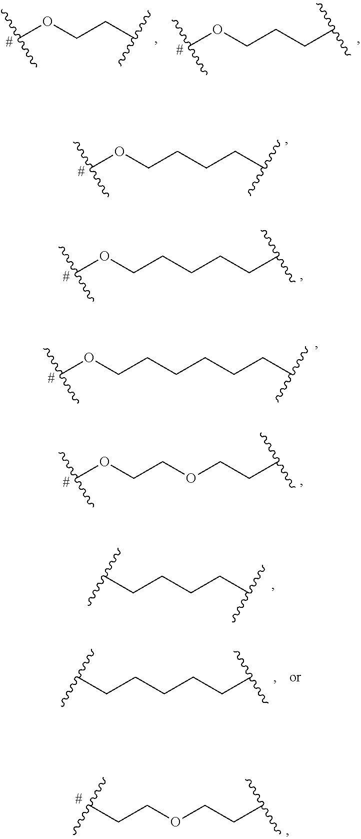
wherein #represents the point of attachment to L1.

38.-45. (canceled)

46. The method of claim 6, wherein X² is:
covalent bond, 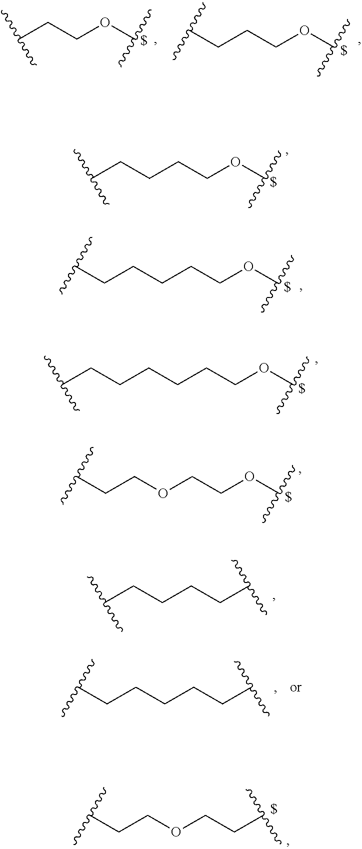
wherein $ represents the point of attachment to L2.

47.-50. (canceled)

51. The composition or method of claim 19, wherein the compound is selected from: 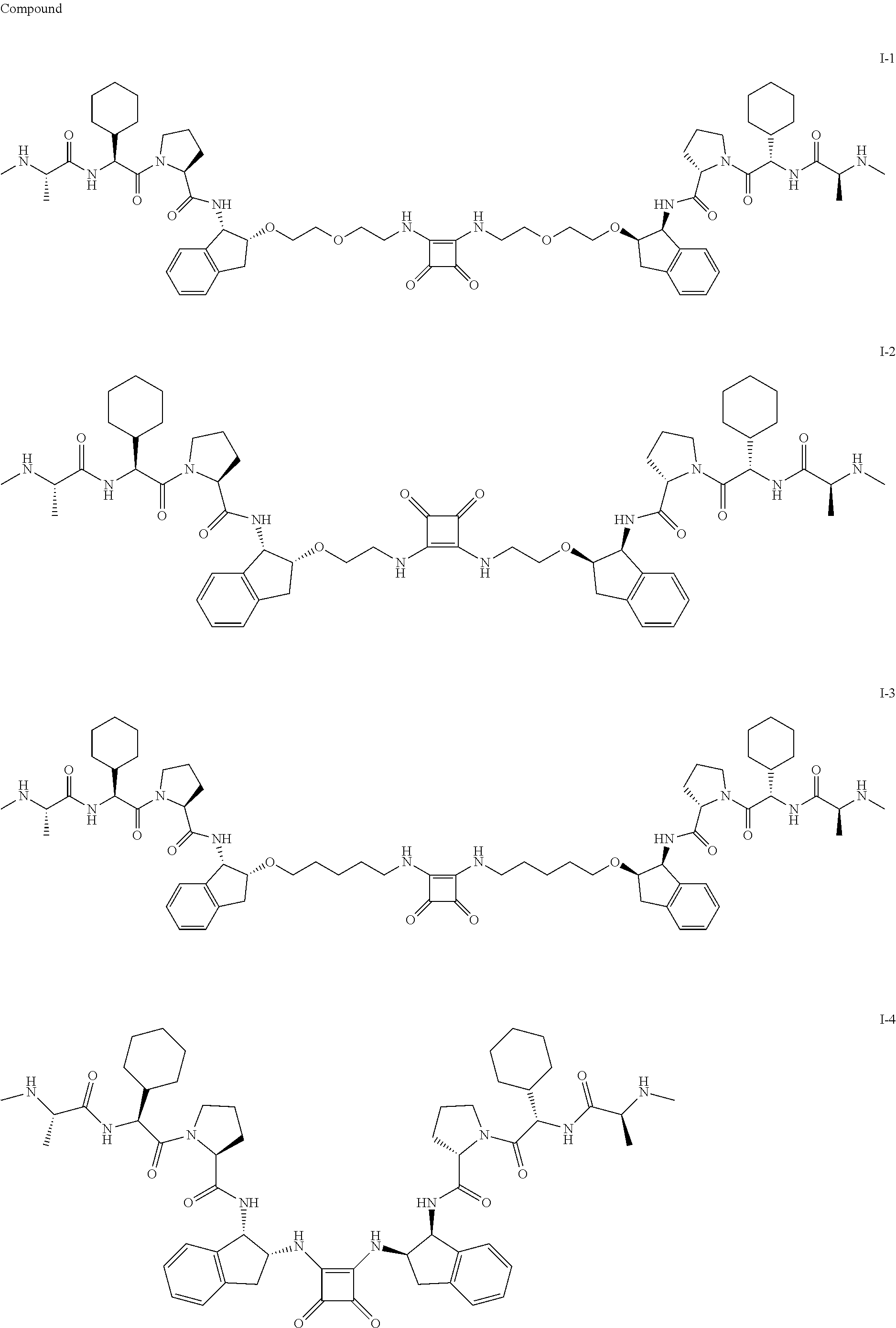 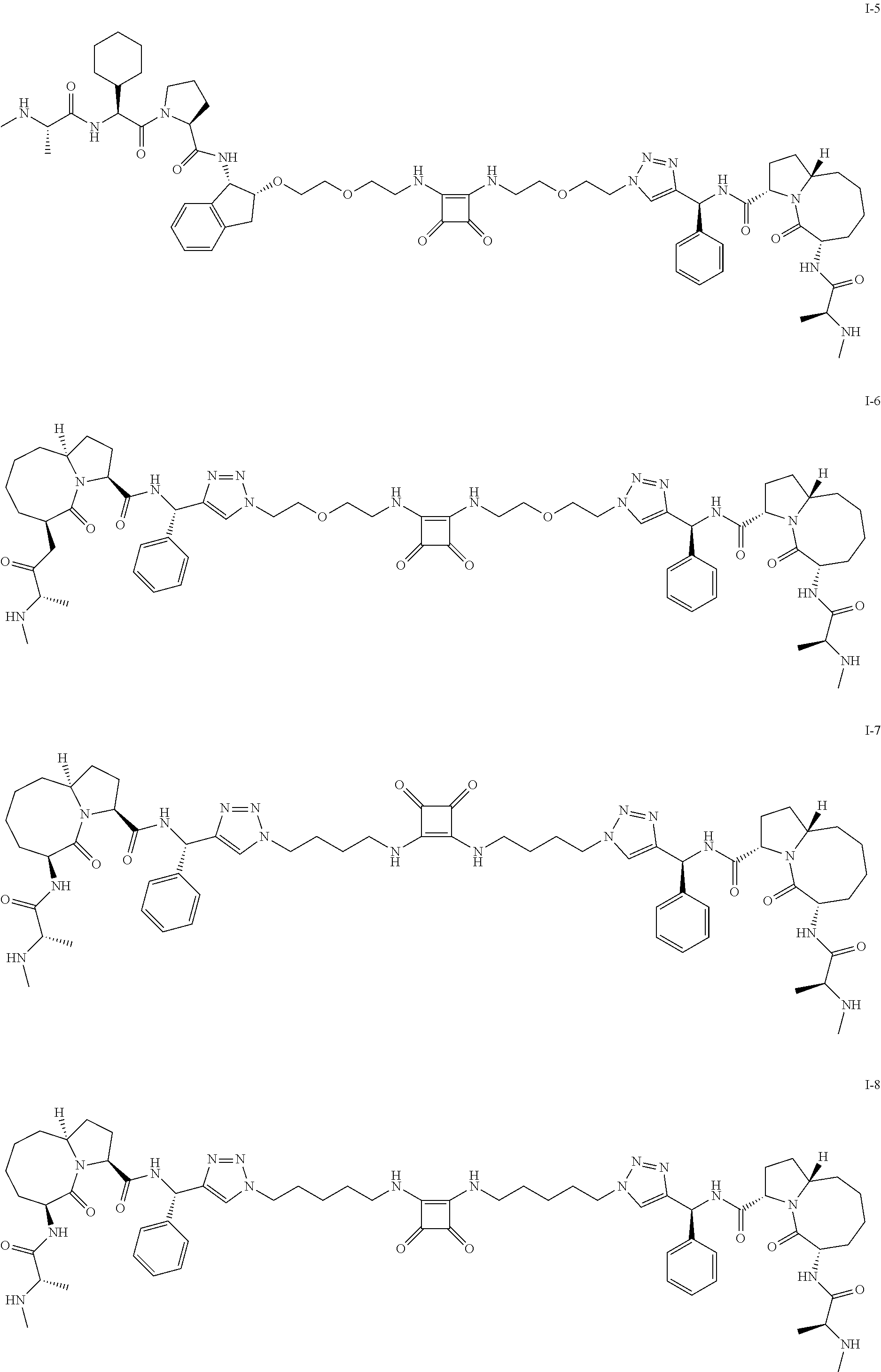 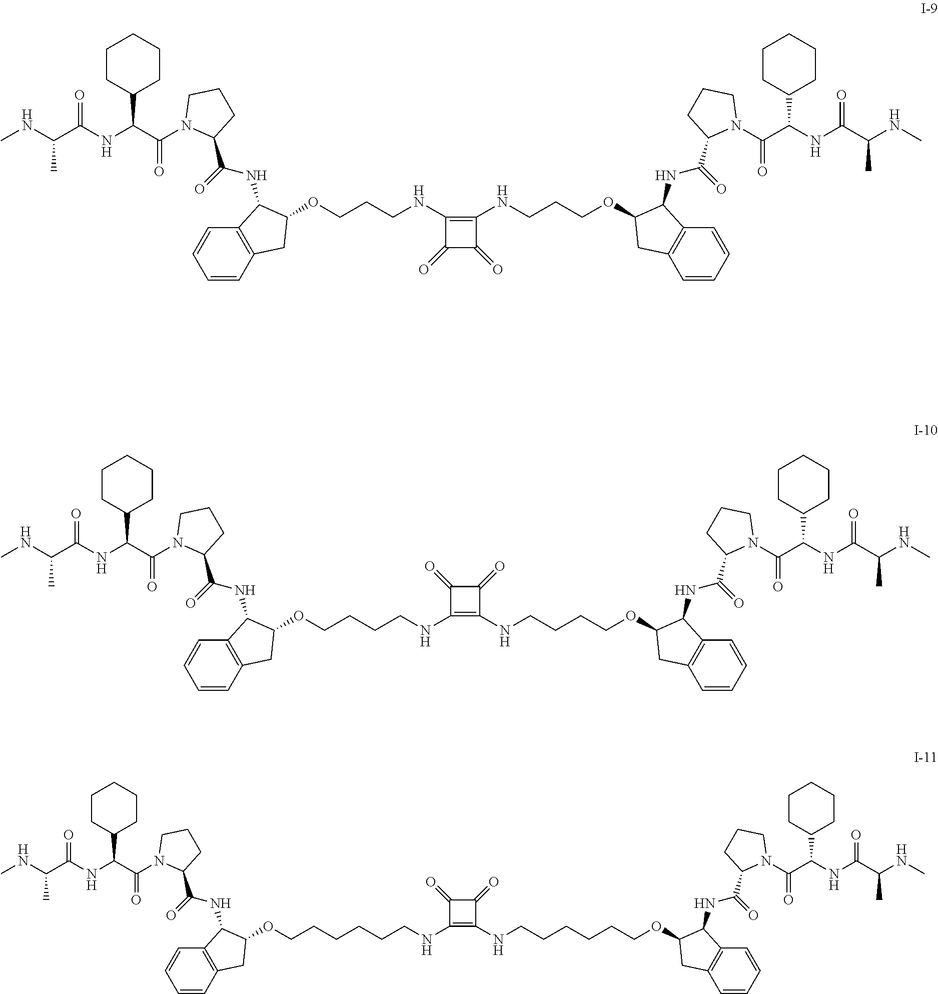
or a pharmaceutically acceptable salt thereof.

52. The method of claim 11, wherein the cancer is bladder cancer, breast cancer, colon cancer, liver cancer, lung cancer, ovarian cancer, esophageal cancer, cholangiocarcinoma, glioblastoma, medulloblastoma, pancreatic cancer, or prostate cancer.

53. The method of claim 10, wherein the pulmonary disease or disorder is chronic obstructive pulmonary disease (COPD), cystic fibrosis, idiopathic pulmonary fibrosis or COVID-19.

54. A pharmaceutical composition comprising an IAP inhibitor for use in combination with a cellular kinase inhibitor
wherein the IAP inhibitor is a compound of formula I: 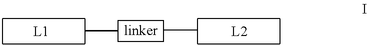
or a pharmaceutically acceptable salt thereof,
wherein:
L1 is a first ligand;
L2 is a second ligand; and
linker is a bivalent linker comprising 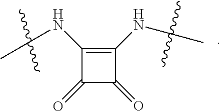 [SMILES: *Nc1c(N*)c(=O)c1=O]
